(19) United States
(12) Patent Application Publication
(10) Pub. No.: US 2025/0229057 A1
(43) Pub. Date: Jul. 17, 2025

(54) EXPANDABLE SHEATH

(71) Applicant: EDWARDS LIFESCIENCES CORPORATION, Irvine, CA (US)

(72) Inventors: ROY SHITRIT, Zichron Yaakov (IL); Amir Davidesko, Binyamina Givat Ada (IL)

(21) Appl. No.: 18/411,286

(22) Filed: Jan. 12, 2024

Related U.S. Application Data
(63) Continuation of application No. PCT/US2022/038481, filed on Jul. 27, 2022.
(60) Provisional application No. 63/324,436, filed on Mar. 28, 2022.
(60) Provisional application No. 63/230,631, filed on Aug. 6, 2021.

(51) Int. Cl. A61M 25/00; A61F 2/24

(52) U.S. Cl. CPC A61M 25/0023; A61F 2/2436; A61M 25/0045; A61M 2025/0024

(57) ABSTRACT

The expandable sheaths disclosed herein have a plurality of radially arranged layers, including an inner liner, a first polymeric layer positioned radially outward of the inner liner, a braided layer positioned radially outward of the first polymeric layer, a second polymeric layer positioned radially outward of the braided layer, and an outer liner positioned radially outward of the second polymeric layer. The sheath comprises a plurality of longitudinally-extending pleats in a collapsed (unexpanded) configuration, and the longitudinally-extending pleats incorporate the plurality of radially arranged layers. When a medical device is passed through the sheath, a diameter of the sheath locally expands around the medical device while the first and second polymeric layers resist axial elongation of the sheath such that a length of the sheath remains substantially constant. The sheath resiliently collapses after the passage of the medical device.

### CROSS-REFERENCE TO RELATED APPLICATIONS

This application is a continuation of PCT patent application No. PCT/US2022/038481, filed on Jul. 27, 2022, that claims the benefit of U.S. Provisional Application No. 63/230,631, filed Aug. 6, 2021, and U.S. Provisional Application No. 63/324,436, filed Mar. 28, 2022, the contents of which are incorporated herein by reference in their entirety.

### FIELD

The present application relates to expandable introducer sheaths for prosthetic devices such as transcatheter heart valves and methods of making the same.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss.

### SUMMARY

Aspects of the present expandable sheath can minimize trauma to the vessel by allowing for temporary expansion of a portion of the introducer sheath to accommodate a delivery system, followed by a return to the original diameter once the delivery system passes through. Some aspects can comprise a sheath with a smaller profile than prior art introducer sheaths. Furthermore, certain aspects can reduce the length of time a procedure takes and the risk of a longitudinal or radial vessel tear or plaque dislodgement because only one sheath is required rather than several different sizes. Aspects of the present expandable sheath can require only a single vessel insertion instead of requiring multiple insertions to dilate the vessel.

According to some aspects, a sheath for introducing a prosthetic device wherein the sheath comprises an inner liner and an outer layer is disclosed. At least a portion of the sheath can be designed or configured to locally expand from a first diameter (rest diameter) to a second diameter (expanded diameter) as the prosthetic device is pushed through a lumen of the sheath, and then at least partially return to the first diameter once the prosthetic device has passed through.

According to some aspects, an expandable sheath for deploying a medical device is disclosed. The expandable sheath has a proximal end and a distal end, an inner surface and an outer surface and comprising: an inner liner comprising one or more polymer layers; wherein the inner liner has a first surface and an opposite second surface, wherein the first surface of the inner liner defines the inner surface of the sheath; a first polymeric layer surrounding radially outward of the inner liner, such that it is positioned at the second surface of the inner liner and wherein the first polymeric layer comprises one or more sublayers; a braided layer disposed radially outward of the first polymeric layer; a second polymeric layer surrounding radially outward of the braided layer, wherein the second polymeric layer comprises one or more sublayers; an outer liner comprising one or more polymer layers; wherein the outer liner has a first surface and an opposite second surface, wherein the first surface of the outer liner overlies the second polymeric layer, and wherein the second surface of the liner layer defines the outer surface of the sheath; wherein the inner liner, the first polymeric layer, the second polymeric layer and the outer liner form a laminate structure; and wherein when a medical device is passed through the sheath, a diameter of the sheath locally expands from a first unexpended diameter around the medical device to a second expanded diameter, while the first and second polymeric layers resist axial elongation of the sheath such that a length of the sheath remains substantially constant; and wherein the sheath resiliently returns to a third diameter after the passage of the medical device.

In some implementations, the first polymeric layer can be provided as a film or as a coating. In some aspects, the second polymeric layer can be provided as a film or as a coating.

Also disclosed are implementations where a portion of the proximal end of the expandable sheath further comprises a third polymeric layer comprising one or more layers and surrounding radially outward of the outer layer.

According to some aspects, the sheath comprises a plurality of longitudinally extending pleats. In such exemplary and unlimiting aspects, the plurality of pleats can extend around at least a portion of a circumference of the sheath. In addition or in the alternative, disclosed are aspects where the plurality of pleats can form a plurality of circumferentially spaced ridges and a plurality of circumferentially spaced valleys and wherein, as the medical device is passed through the sheath, the ridges and valleys at least partially level out to allow a sheath wall to radially expand.

Also disclosed are aspects of an expandable sheath for deploying a medical device having a proximal end and a distal end, an inner surface and an outer surface and comprising: an inner liner comprising one or more polymer layers; wherein the inner liner has a first surface and an opposite second surface, wherein the first surface of the inner liner defines the inner surface of the sheath; a first polymeric layer surrounding radially outward of the inner liner, such that it is positioned at the second surface of the inner liner and wherein the first polymeric layer comprises one or more sublayers; a braided layer disposed radially outward of the first polymeric layer; a second polymeric layer surrounding radially outward of the braided layer, wherein the second polymeric layer comprises one or more sublayers; an outer liner comprising one or more polymer layers; wherein the outer liner has a first surface and an opposite second surface, wherein the first surface of the outer liner overlies the second polymeric layer, and wherein the second surface of the liner layer defines the outer surface of the sheath; wherein the inner liner, the first polymeric layer, the second polymeric layer and the outer liner form a laminate structure; wherein the inner liner, the first polymeric layer, the second polymeric layer and the outer liner form a laminate structure; and wherein when a medical device is passed through the sheath, the diameter of the sheath locally expands from a first unexpended diameter around the medical device to a second expanded diameter, while the first and second polymeric layers resist axial elongation of the sheath such that a length of the sheath remains substantially constant; and wherein the sheath resiliently returns to a third diameter after the passage of the medical device.

Also disclosed herein are methods of making an expandable sheath, the method comprising: forming an inner liner; wherein the inner liner comprises one or more polymer layers and wherein the inner liner has a first surface and an opposite second surface; forming a first polymeric layer, wherein the first polymeric layer is positioned radially outward of the inner liner; wherein the first polymeric layer comprises one or more sublayers; and wherein the first polymeric layer overlies the second surface of the inner liner; positioning a braided layer radially outward of a first polymeric layer; forming a second polymeric layer such that it is positioned radially outward of the braided layer; wherein the second polymeric layer comprises one or more sublayers; forming an outer liner radially outward of the second polymeric layer; wherein the outer liner comprises one or more polymer layers; and wherein the outer liner has a first surface and an opposite second surface and wherein the first surface of the outer liner is in contact with at least a portion of the second polymeric layer; heating the inner liner, the first polymeric layer, the braided layer, the second polymeric layer, and the outer liner to form a laminate structure; and crimping the laminate structure to form a plurality of longitudinally-extending pleats, wherein the plurality of longitudinally-extending pleats are configured to expand upon passage of a medical device through the sheath laminate.

According to some aspects, a method of making an expandable sheath is disclosed, the method comprising: forming an inner liner; wherein the inner liner comprises one or more polymer layers and wherein the inner liner has a first surface and an opposite second surface; forming a first polymeric layer, wherein the first polymeric layer is positioned radially outward of the inner liner; wherein the first polymeric layer comprises one or more sublayers; and wherein the first polymeric layer overlies the second surface of the inner liner; positioning a braided layer radially outward of a first polymeric layer; forming a second polymeric layer such that it is positioned radially outward of the braided layer; wherein the second polymeric layer comprises one or more sublayers; forming an outer liner radially outward of the second polymeric layer; wherein the outer liner comprises one or more polymer layers, and wherein the outer liner has a first surface and an opposite second surface, and wherein the first surface of the outer liner is in contact with at least a portion of the second polymeric layer; heating the inner liner, the first polymeric layer, the braided layer, the second polymeric layer, and the outer liner to form a laminate structure; and crimping the laminate structure to form a plurality of longitudinally-extending pleats, wherein the plurality of longitudinally-extending pleats are configured to expand upon passage of a medical device through the sheath; and then forming a third polymeric layer radially outward of at least a portion of the outer liner; wherein the third polymeric layer comprises one or more polymer layers.

In some aspects disclosed is also a method of delivering a prosthetic device to a procedure site, the method comprising inserting an expandable sheath at least partially into the vasculature of the patient, the expandable sheath comprising a plurality of radially arranged layers including an inner liner, a first polymeric layer radially outward of the inner liner, a braided layer radially outward of the first polymeric layer, a second polymeric layer radially outward of the braided layer, and an outer liner, and wherein the sheath comprises a plurality of longitudinally-extending pleats; advancing a medical device through an inner lumen defined by a first surface of the inner liner of the sheath, the medical device applying an outward radial force on the inner liner of the sheath; locally expanding the sheath from an unexpanded state to a locally expanded state; at least partially unfolding the plurality of longitudinally-extending pleats during a local expansion of the sheath, wherein each of the plurality of longitudinally-extending pleats incorporates at least a portion of the plurality of radially arranged layers; locally collapsing the sheath from the locally expanded state at least partially back to the unexpanded state after passage of the medical device.

According to some aspects, disclosed is a method of delivering a prosthetic device to a procedure site, the method comprising: inserting an expandable sheath at least partially into the vasculature of the patient, the expandable sheath comprising a plurality of radially arranged layers including an inner liner, a first polymeric layer radially outward of the inner liner, a braided layer radially outward of the first polymeric layer, a second polymeric layer radially outward of the braided layer, an outer liner, and a third polymer layer disposed radially outward of at least a portion of the outer liner, and wherein the sheath comprises a plurality of longitudinally-extending pleats; advancing a medical device through an inner lumen defined by a first surface of the inner liner of the sheath, the medical device applying an outward radial force on the inner liner of the sheath; locally expanding the sheath from an unexpanded state to a locally expanded state; at least partially unfolding the plurality of longitudinally-extending pleats during a local expansion of the sheath, wherein each of the plurality of longitudinally-extending pleats incorporates at least a portion of the plurality of radially arranged layers; and locally collapsing the sheath from the locally expanded state at least partially back to the unexpanded state after passage of the medical device.

Aspects of the disclosure will be set forth, in part, in the detailed description, figures, and claims which follow, and in part will be derived from the detailed description or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

FIG. 1 illustrates a delivery system for a cardiovascular prosthetic device, according to one aspect.

FIG. 2 illustrates an expandable sheath that can be used in combination with the delivery system of FIG. 1, according to one aspect.

FIG. 3 is a magnified view of a portion of the expandable sheath of FIG. 2.

FIG. 4 is a side elevation cross-sectional view of a portion of the expandable sheath of FIG. 2.

FIG. 5A is a magnified view of a portion of the expandable sheath of FIG. 2, with the outer layer removed for illustration purposes.

FIG. 5B is a magnified view of a portion of the braided layer of the sheath of FIG. 2.

FIG. 6 is a magnified view of a portion of the expandable sheath of FIG. 2, illustrating the expansion of the sheath as a prosthetic device is advanced through the sheath.

FIG. 7 is a magnified, partial cross-sectional view illustrating the constituent layers of the sheath of FIG. 2 disposed on a mandrel.

FIG. 8 is a magnified view illustrating some aspects of an expandable sheath.

FIG. 9 is a cross-sectional view of an apparatus that can be used to form an expandable sheath according to one aspect.

FIGS. 10A-10D illustrate some aspects of a braided layer in which the filaments of the braided layer are configured to buckle when the sheath is in a radially collapsed state.

FIG. 11 shows a side cross-sectional view of an assembly of an expandable sheath with a vessel dilator.

FIG. 12 shows the vessel dilator of the assembly aspect of FIG. 11.

FIG. 13 shows a side view of some aspects of an assembly, including an expandable sheath and a vessel dilator.

FIG. 14 shows a side view of the assembly aspect of FIG. 13, with the vessel dilator, pushed partially away from the expandable sheath.

FIG. 15 shows a side view of the assembly aspect of FIG. 13, with the vessel dilator, pushed fully away from the expandable sheath.

FIG. 16 shows a side view of the assembly aspect of FIG. 13, with the vessel dilator being retracted into the expandable sheath.

FIG. 17 shows a side view of the assembly aspect of FIG. 13, with the vessel dilator being retracted further into the expandable sheath.

FIG. 18 shows a side view of the assembly aspect of FIG. 13, with the vessel dilator being fully retracted into the expandable sheath.

FIG. 19 shows a side cross-sectional view of an exemplary assembly aspect, including an expandable sheath and a vessel dilator.

FIG. 20 illustrates an aspect of a vessel dilator that may be used in combination with the expandable sheaths described herein.

FIG. 21 illustrates an aspect of a vessel dilator that may be used in combination with the expandable sheaths described herein.

FIG. 22 shows a side view with a cutaway to cross section of an aspect of an expandable sheath having an outer cover and an overhang.

FIG. 23 shows an example aspect of an outer cover having longitudinal scorelines.

FIG. 24 illustrates an end portion of an aspect of a braided layer of an expandable sheath.

FIG. 25A illustrates a perspective view of a roller-based crimping mechanism aspect for crimping an expandable sheath.

FIG. 25B illustrates a side view of a disc-shaped roller and connector of the crimping mechanism shown in FIG. 25A.

FIG. 25C illustrates a top view of a disc-shaped roller and connector of the crimping mechanism shown in FIG. 25A.

FIG. 26 shows an aspect of a device for crimping an elongated expandable sheath. The encircled portion of the device is magnified in the inset on the left side of the picture.

FIG. 27 shows an aspect of an expandable sheath having an inner layer with scorelines.

FIG. 28 shows an example aspect of a braided layer of an expandable sheath.

FIG. 29 shows a perspective view of an example expandable sheath aspect.

FIG. 30 shows a perspective view of the aspect of FIG. 29, with the outer heat shrink tubing layer partially torn away from the inner sheath layers.

FIG. 31 shows a side view of a sheath aspect prior to the movement of a delivery system therethrough.

FIG. 32 shows a side view of a sheath aspect as a delivery system moves through, splitting the heat-shrink tubing layer.

FIG. 33 shows a side view of a sheath aspect with the delivery system fully moved through, the heat shrink tubing layer fully split along the length of the sheath.

FIG. 34 shows a perspective view of a sheath aspect having a distal end portion folded around an introducer.

FIG. 35 shows an enlarged, cross-sectional view of the distal end portion folded around the introducer.

FIG. 36 shows a cross section of an example expandable sheath aspect.

FIG. 37 shows an aspect of a cushioning layer.

FIG. 38 shows an exemplary aspect of a cushioning layer.

FIG. 39 shows a side view of an example expandable sheath aspect.

FIG. 40 shows a longitudinal cross section of the aspect of FIG. 39.

FIG. 41 shows a transverse cross section of an example expandable sheath aspect.

FIG. 42 shows a partial longitudinal cross section of an example expandable sheath aspect.

FIG. 43 shows a transverse cross section of an example expandable sheath aspect in an expanded state.

FIG. 44 shows a transverse cross section of the expandable sheath aspect of FIG. 43 during the crimping process.

FIG. 45 shows a perspective view of a sheath aspect similar to the sheath of FIG. 43, in the expanded state.

FIG. 46 shows a perspective view of a sheath aspect similar to the sheath of FIG. 43, in the folded and compressed state.

FIG. 47 shows an example aspect of a braided layer.

FIG. 48 shows a cross-section of an expandable sheath aspect.

FIG. 49 shows a simplified cross-section of a sheath wall having a plurality of longitudinally extending pleats in one aspect.

FIGS. 50A-50B show a photograph of the sheath in a collapsed state (FIG. 50A) and an expanded state (FIG. 50B).

FIG. 51 shows a simplified cross-section of a distal portion of a sheath wall having a plurality of longitudinally extending pleats in one aspect.

FIG. 52 shows a simplified cross-section of a distal portion of a sheath wall having a plurality of longitudinally extending pleats in one aspect.

FIG. 53 shows a flow chart showing example fabrication steps for an expandable sheath.

FIG. 54 shows a cross-section of an expandable sheath aspect in a proximal portion of the sheath in one aspect.

FIG. 55 shows a side view of the sheath of FIG. 54.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

The present disclosure relates to introducer sheaths. Such introducer sheaths may be radially expandable. However, currently known sheaths tend to have complex mechanisms, such as ratcheting mechanisms that maintain the sheath in an expanded configuration once a device with a larger diameter than the sheath's original diameter is introduced. Existing expandable sheaths can also be prone to axial elongation as a consequence of the application of longitudinal force attendant to passing a prosthetic device through the sheath. Such elongation can cause a corresponding reduction in the diameter of the sheath, increasing the force required to insert the prosthetic device through the narrowed sheath.

Accordingly, there remains a need in the art for an improved introducer sheath for endovascular systems used for implanting valves and other prosthetic devices.

### Definitions

As used in this application and the claims, the singular forms “a,” “an,” and “the” include the plural forms unless the context clearly dictates otherwise. Thus, for example, reference to a “polymer” includes aspects having two or more such polymers unless the context clearly indicates otherwise.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable combination.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and the claims, the term “comprising” can include the aspects “consisting of” and “consisting essentially of.” Additionally, the term “includes” means “comprises.”

For the terms “for example” and “such as” and grammatical equivalences thereof, the phrase “and without limitation” is understood to follow unless explicitly stated otherwise.

References in the specification and concluding claims to parts by weight of a particular element or component in a composition or article denote the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5 and are present in such ratio regardless of whether additional components are contained in the composition.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, including the recited values, may be used. Further, ranges can be expressed herein as from “about” one particular value and/or to “about” another particular value. When such a range is expressed, some aspects include from one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

As used herein, the term “and/or” includes any and all combinations of one or more of the associated listed items. It is also understood that the term “and/or” includes where one or another of the associated listed items is present and the aspects where both of the associated listed items are present or any combinations of the associated listed items are present.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “substantially” means that the subsequently described event or circumstance completely occurs or that the subsequently described event or circumstance generally, typically, or approximately occurs.

As used herein, the term “substantially,” when used in reference to a composition or a compound, refers to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” in, for example, the context “substantially free” refers to a composition having less than about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component.

Further, the terms “coupled” and “associated” generally mean electrically, electromagnetically, and/or physically (e.g., mechanically or chemically) coupled or linked and do not exclude the presence of intermediate elements between the coupled or associated items.

It will be understood that, although the terms “first,” “second,” etc., may be used herein to describe various elements, components, regions, layers sections, and/or steps. These elements, components, regions, layers, sections, and/or steps should not be limited by these terms. These terms are only used to distinguish one element, component, region, layer, or section from another element, component, region, layer, section, or step. Thus, a first element, component, region, layer, section, or step discussed below could be termed a second element, component, region, layer, section, or step without departing from the teachings of example aspects.

It is understood that the terms “layer” and “liner” can be used interchangeably. For example, the aspects describing an “inner liner” also include aspects describing an “inner layer.” Similarly, the aspects describing an “outer layer” also include aspects describing an “outer liner.”

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “atraumatic” is commonly known in the art and refers to a device or a procedure that minimizes tissue injury.

Some of the aspects disclosed herein comprise a plurality of longitudinally-extending creases. It is understood that the terms “creases,” “folds,” and “pleats” as used in reference to these aspects can be used interchangeably. It is understood that the pleats or creases can be arranged in a specific pattern, or they can be randomly formed along a length of the sheath. For example, pleats formed along the length of the sheath are formed as a result of the manufacturing process where the various polymer layers encapsulate a braid (or braided layer; it is understood that braid and braided layer can be used interchangeably) and form creases that can be flattened out during expansion process of the valve. In some aspects, pleats can have an arranged pattern. For example, and without limitation, pleats can have an arranged pattern at a tip of the sheath. In some implementations, the pleats can have an even area in an arc length of a cross section. In such aspects, each of the formed pleats can have a substantially identical area, whether there are 2 pleats, or 3 pleats, or 4 pleats, or 5 pleats, and so on. In some implementations, the pleats can have a predetermined design for the desired application.

It is understood that the term “collapsed” as used herein, refers to a natural unexpanded state of the sheath.

Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

While aspects of the present disclosure can be described and claimed in a particular statutory class, such as the system statutory class, this is for convenience only, and one of ordinary skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present disclosure may be understood more readily by reference to the following detailed description of various aspects of the disclosure and the examples included wherein and to the Figures and their previous and following description.

The present disclosure may be understood more readily by reference to the following detailed description of various aspects of the disclosure and the examples included wherein and to the Figures and their previous and following description.

The expandable introducer sheaths described herein can be used to deliver a prosthetic device through a patient's vasculature to a procedure site within the body. The sheath can be constructed to be highly expandable and collapsible in the radial direction while limiting axial elongation of the sheath and, thereby, undesirable narrowing of the lumen. In one aspect, the expandable sheath includes a braided layer, one or more relatively thin, non-elastic polymeric layers, and an elastic layer. The sheath can resiliently expand from its natural diameter to an expanded diameter as a prosthetic device is advanced through the sheath and can return to its natural diameter upon passage of the prosthetic device under the influence of the elastic layer. In certain aspects, the one or more polymeric layers can engage the braided layer and can be configured to allow radial expansion of the braided layer while preventing axial elongation of the braided layer, which would otherwise result in elongation and narrowing of the sheath.

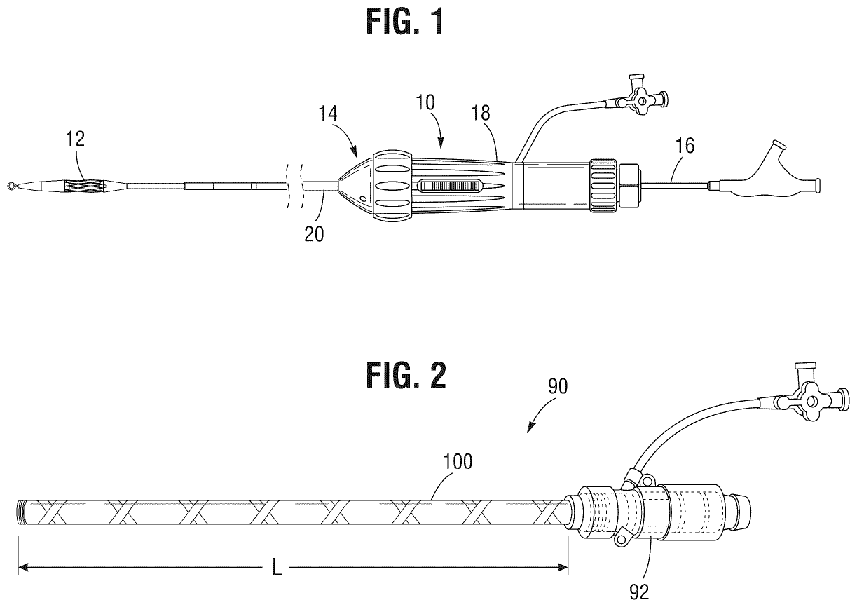

FIG. 1 illustrates a representative delivery apparatus 10 for delivering a medical device, such as a prosthetic heart valve or other prosthetic implant, to a patient. The delivery apparatus 10 is exemplary only and can be used in combination with any of the expandable sheath aspects described herein. Likewise, the sheaths disclosed herein can be used in combination with any of the various known delivery apparatuses. The delivery apparatus 10 illustrated can generally include a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate the delivery and positioning of a prosthetic heart valve 12 at an implantation site in a patient's body. The guide catheter 14 includes a handle portion 18 and an elongated guide tube or shaft 20 extending from the handle portion 18.

The prosthetic heart valve 12 can be delivered into a patient's body in a radially compressed configuration and radially expanded to a radially expanded configuration at the desired deployment site. In the illustrated aspect, the prosthetic heart valve 12 is a plastically expandable prosthetic valve that is delivered into a patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Some details regarding a plastically expandable heart valve that can be implanted using the devices disclosed herein are disclosed in U.S. Publication No. 2012/0123529, which is incorporated herein by reference. In some aspects, the prosthetic heart valve 12 can be a self-expandable heart valve that is restrained in a radially compressed configuration by a sheath or other component of the delivery apparatus and self-expands to a radially expanded configuration when released by the sheath or other component of the delivery apparatus. Some details regarding a self-expandable heart valve that can be implanted using the devices disclosed herein are disclosed in U.S. Publication No. 2012/0239142, which is incorporated herein by reference. In some aspects, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve.

Some details regarding a mechanically expandable heart valve that can be implanted using the devices disclosed herein are disclosed in U.S. Publication No. 2018/0153689, which is incorporated herein by reference. In some aspects, a prosthetic valve can incorporate two or more of the above-described technologies. For example, a self-expandable heart valve can be used in combination with an expansion device to assist in the expansion of the prosthetic heart valve.

FIG. 2 illustrates an assembly 90 (which can be referred to as an introducer device or assembly) that can be used to introduce the delivery apparatus 10 and the prosthetic device 12 into a patient's body, according to one aspect. The introducer device 90 can comprise a housing 92 at a proximal end of the device and an expandable sheath 100 extending distally from the housing 92. The housing 92 can function as a handle for the device. The expandable sheath 100 has a central lumen 112 (FIG. 4) to guide the passage of the delivery apparatus for the prosthetic heart valve. Generally, during use, a distal end of the sheath 100 is passed through the patient's skin and inserted into a vessel, such as the femoral artery. The delivery apparatus 10 with its implant 12 can then be inserted through the housing 92 and the sheath 100 and advanced through the patient's vasculature to the treatment site, where the implant is to be delivered and implanted within the patient. In certain aspects, the introducer housing 92 can include a hemostasis valve that forms a seal around the outer surface of the guide catheter 14 once inserted through the housing to prevent leakage of pressurized blood.

In alternative aspects, the introducer device 90 need not include a housing 92. For example, the sheath 100 can be an integral part of a component of the delivery apparatus 10, such as the guide catheter. For example, the sheath can extend from the handle 18 of the guide catheter. Some examples of introducer devices and expandable sheaths can be found in U.S. patent application Ser. No. 16/378,417 and U.S. Provisional Patent Application No. 62/912,569, which are incorporated by reference in their entireties.

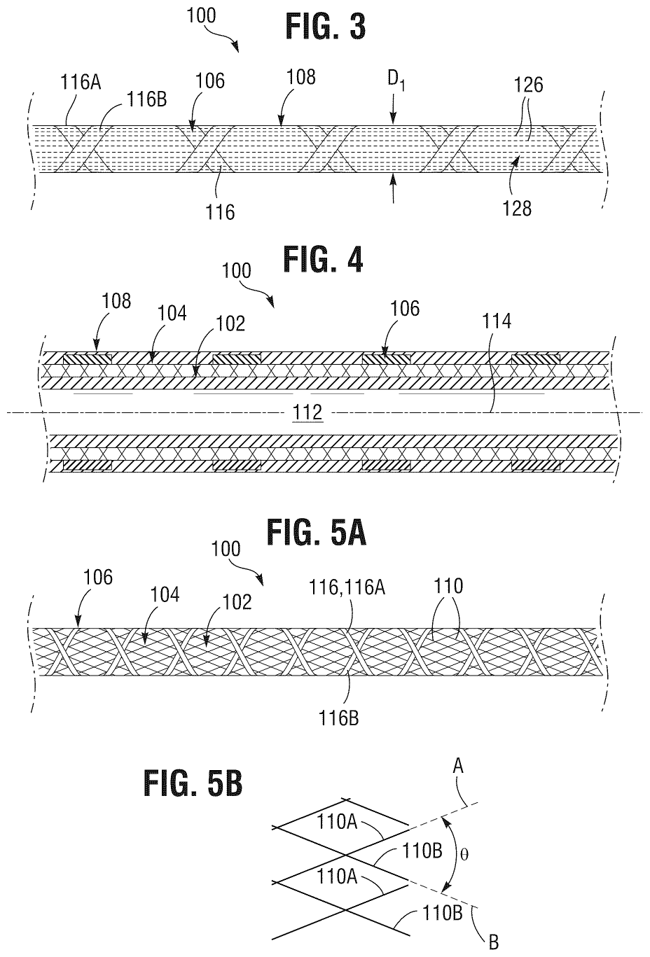

FIG. 3 illustrates the expandable sheath 100 in greater detail. With reference to FIG. 3, the sheath 100 can have a natural, unexpanded outer diameter D₁. In certain aspects, the expandable sheath 100 can comprise a plurality of coaxial layers extending along at least a portion of the length L of the sheath (FIG. 2). For example, with reference to FIG. 4, the expandable sheath 100 can include a first layer 102 (also referred to as an inner layer or an inner liner), a second layer 104 disposed around and radially outward of the first layer 102, a third layer 106 disposed around and radially outward of the second layer 104, and a fourth layer 108 (also referred to as an outer layer or an outer liner) disposed around and radially outward of the third layer 106. In the illustrated configuration, the inner layer (liner) 102 can define the lumen 112 of the sheath extending along a central axis 114.

Referring to FIG. 3, when the sheath 100 is in an unexpanded state, the inner layer (liner) 102 and/or the outer layer (liner) 108 can form longitudinally-extending folds or pleats or creases such that the surface of the sheath comprises a plurality of ridges 126 (also referred to herein as “folds” or “pleats”). The ridges 126 can be circumferentially spaced apart from each other by longitudinally-extending valleys 128. When the sheath expands beyond its natural (initial) diameter D₁, the ridges 126 and the valleys 128 can level out or be taken up as the surface radially expands and the circumference increases, as described below. When the sheath collapses back to its natural diameter (or, in other words, returns to its unexpanded state), the ridges 126 and valleys 128 can reform.

In certain aspects, the inner layer (liner) 102 and/or the outer layer (liner) 108 can comprise a relatively thin layer of polymeric material. For example, in some aspects, the thickness of the inner layer 102 can be from 0.01 mm to 0.5 mm, 0.02 mm to 0.4 mm, or 0.03 mm to 0.25 mm. In certain aspects, the thickness of the outer layer 108 can be from 0.01 mm to 0.5 mm, 0.02 mm to 0.4 mm, or 0.03 mm to 0.25 mm. In yet some aspects, the inner liner 102 and the outer liner 108 can comprise at least one polymer layer. In some aspects, the inner liner and the outer liner each can comprise two or more layers of polymeric material.

In certain examples, the inner layer 102 and/or the outer layer 108 can comprise a lubricious, low-friction, and/or relatively non-elastic material. In particular aspects, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such a low coefficient of friction materials can facilitate the passage of the prosthetic device through the lumen 112. Some suitable materials for the inner and outer layers can include polytetrafluoroethylene (PTFE), expanded polytetrafluoroethylene (ePTFE), ethylene tetrafluoroethylene (ETFE), nylon, polyethylene (such as, for example, low-density polyethylene (LDPE), high density polyethylene (HDPE)), polyether block amide (e.g., Pebax), bi-oriented polypropylene, cast polypropylene, thermoplastic polyurethane, and/or combinations of any of the above. Some aspects of a sheath 100 can include an additional lubricious liner on the inner surface of the inner layer 102. Examples of such suitable lubricious liners include materials that can further reduce the coefficient of friction of the inner layer 102, such as PTFE, polyethylene (such as, for example, HMWPE, UHMWPE, LDPE, HDPE), polyvinylidine fluoride, and combinations thereof. Suitable materials for a lubricious liner also include some materials desirably having a coefficient of friction of 0.1 or less.

Some aspects of the sheath 100 can include an exterior hydrophilic coating on the outer surface of the outer layer (liner) 108. Such a hydrophilic coating can facilitate the insertion of the sheath 100 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N. V, Heerlen, the Netherlands), as well as other hydrophilic coatings (e.g., PTFE, polyethylene, polyvinylidine fluoride), are also suitable for use with the sheath 100. Such hydrophilic coatings may also be included on the inner surface of the inner layer 102 to reduce friction between the sheath and the delivery system, thereby facilitating the use and improving safety. In some aspects, a hydrophobic coating, such as Perylene, may be used on the outer surface of the outer layer 108 or the inner surface of the inner layer 102 in order to reduce friction.

In certain aspects, the second layer 104 can be braided. FIGS. 5A and 5B illustrate the sheath 100 with the outer layer 108 removed to expose the elastic layer 106. With reference to FIGS. 5A and 5B, the braided layer 104 can comprise a plurality of members or filaments 110 (e.g., metallic or synthetic wires or fibers) braided together. The braided layer 104 can have any desired number of filaments 110, which can be oriented and braided together along any suitable number of axes. For example, with reference to FIG. 5B, the filaments 110 can include a first set of filaments 110A oriented parallel to a first axis A and a second set of filaments 110B oriented parallel to a second axis B. The filaments 110A and 110B can be braided together in a biaxial braid such that filaments 110A oriented along axis A form an angle θ with the filaments 110B oriented along axis B. In certain aspects, the angle θ can be from 5° to 70°, 10° to 60°, 10° to 50°, or 10° to 45°. In the illustrated aspect, the angle θ is 45°; however, it is understood that this is for exemplary purposes only and is not limiting. In some implementations, the filaments 110 can also be oriented along three axes and braided in a triaxial braid or oriented along any number of axes and braided in any suitable braid pattern.

The braided layer 104 can extend along substantially the entire length L of the sheath 100, or alternatively, it can extend only along a portion of the length of the sheath. In particular aspects, the filaments 110 can be wires made from metal (e.g., Nitinol, stainless steel, etc.) or any of various polymers or polymer composite materials, such as carbon fiber. In certain aspects, the filaments 110 can be round and can have a diameter of from 0.01 mm to 0.5 mm, 0.03 mm to 0.4 mm, or 0.05 mm to 0.25 mm. In some implementations, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for certain aspects. If a braided wire is used, the braid density can be varied. Some aspects have a braid density of from ten picks per inch to eighty picks per inch and can include eight wires, sixteen wires, or up to fifty-two wires in various braid patterns. In some implementations, the second layer 104 can be laser cut from a tube, or laser-cut, stamped, punched, etc., from sheet stock and rolled into a tubular configuration. Layer 104 can also be woven or knitted, as desired. In some aspects, the braided layer can have a weave pattern of, for example, 1×1 (one over, one under), 2×2 (two over, two under), or 2×1 (two over, one under).

A braided layer 104 can comprise any known in the art material that can be provided for the desired expansion of the sheath. For example, and without limitations, the braided layer 104 can comprise Nitinol or some other shape memory metal or material that can exhibit superelastic properties. In such aspects, these materials can have the advantage of allowing for austenitic finishing (AF) at a certain temperature. For example, a nitinol braided layer having AF at 15 degrees Celsius or less allows for its use in relatively cold operating rooms while still exhibiting superelastic properties. In some implementations, the materials used to form the braided layer can exhibit superelastic properties at temperatures at or above about 15 degrees Celsius.

The third layer 106 can be a resilient, elastic layer (also referred to as an elastic material layer). In certain aspects, the elastic layer 106 can be configured to apply force to the underlying layers 102 and 104 in a radial direction (e.g., toward the central axis 114 of the sheath) when the sheath expands beyond its natural diameter by the passage of the delivery apparatus through the sheath. Stated differently, the elastic layer 106 can be configured to apply encircling pressure to the layers of the sheath beneath the elastic layer 106 to counteract expansion of the sheath. The radially inwardly directed force is sufficient to cause the sheath to collapse radially back to its unexpanded state after the delivery apparatus is passed through the sheath. It is understood, however, that layer 106 can be optional. And also described herein are the aspects where this third elastic layer is not present, while all other layers described herein are. It is also understood that this description includes all various combinations of the layers, and unless it is stated otherwise, some of the described herein layers (liners) can be present while others can be absent. In some implementations, and as shown below, additional layers can also be present.

In the illustrated aspect, the elastic layer 106 can comprise one or more members configured as strands, ribbons, or bands 116 helically wrapped around the braided layer 104. For example, in the illustrated aspect, the elastic layer 106 comprises two elastic bands, 116A and 116B, wrapped around the braided layer with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some aspects, the elastic layer can comprise an elastomeric material having a modulus of elasticity of 200 MPa or less. In some aspects, the elastic layer 106 can comprise a material exhibiting an elongation to break of 200% or greater or an elongation to break of 400% or greater. The elastic layer 106 can also take other forms, such as a tubular layer comprising an elastomeric material, a mesh, a shrinkable polymer layer such as a heat-shrink tubing layer, etc. In lieu of, or in addition to, the elastic layer 106, the sheath 100 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In some aspects, the elastic layer 106 can also be radially outward of the polymeric layer 108.

In certain aspects, one or both of the inner layer 102 and/or the outer layer 108 can be configured to resist axial elongation of the sheath 100 when the sheath expands. More particularly, one or both of the inner layer 102 and/or the outer layer 108 can resist stretching against longitudinal forces caused by friction between a prosthetic device and the inner surface of the sheath such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 5B, the filaments 110A and 110B of the braided layer can be allowed to move angularly relative to each other such that the angle θ changes as the sheath expands and contracts. This, in combination with the longitudinal folds 126 in layers 102 and 108, can allow the lumen 112 of the sheath to expand as a prosthetic device is advanced through it.

For example, in some aspects, the inner layer 102 and the outer layer 108 can be heat-bonded during the manufacturing process, such that the braided layer 104 and the elastic layer 106 are encapsulated between the layers 102 and 108. More specifically, in certain aspects, the inner layer 102 and the outer layer 108 can be adhered to each other through the spaces between the filaments 110 of the braided layer 104 and/or the spaces between the elastic bands 116. Layers 102 and 108 can also be bonded or adhered together at the proximal and/or distal ends of the sheath. In certain aspects, layers 102 and 108 are not adhered to the filaments 110. This can allow the filaments 110 to move angularly relative to each other and relative to the layers 102 and 108, allowing the diameter of the braided layer 104, and thereby the diameter of the sheath, to increase or decrease. As the angle θ between the filaments 110A and 110B changes, the length of the braided layer 104 can also change. For example, as the angle θ increases, the braided layer 104 can foreshorten, and as the angle θ decreases, the braided layer 104 can lengthen to the extent permitted by the areas where the layers 102 and 108 are bonded. However, because the braided layer 104 is not adhered to layers 102 and 108, the change in length of the braided layer that accompanies a change in the angle θ between the filaments 110A and 110B does not result in a significant change in the length L of the sheath.

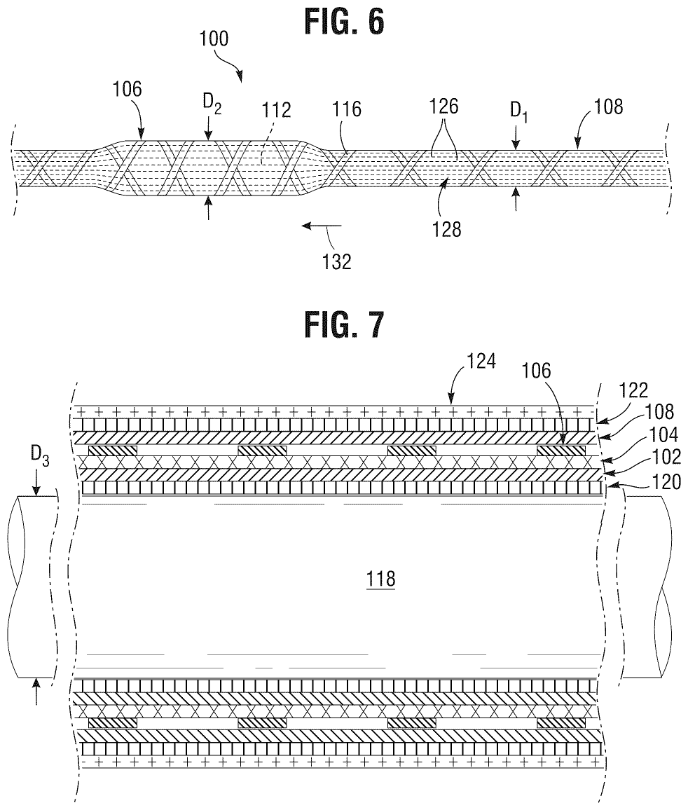

FIG. 6 illustrates a local radial expansion of the sheath 100 as a prosthetic device 12 is passed through the sheath in the direction of arrow 132 (e.g., distally). As the prosthetic device 12 is advanced through the sheath 100, the sheath can resiliently locally expand to a second diameter D₂ that corresponds to the size or diameter of the prosthetic device. As prosthetic device 12 is advanced through the sheath 100, the prosthetic device can apply longitudinal force to the sheath in the direction of motion by virtue of the frictional contact between the prosthetic device and the inner surface of the sheath. However, as noted above, the inner layer (liner) 102 and/or the outer layer (liner) 108 can resist axial elongation such that the length L of the sheath remains constant or substantially constant. This can reduce or prevent the braided layer 104 from lengthening, and thereby constricting the lumen 112.

Meanwhile, the angle θ between the filaments 110A and 110B can increase as the sheath expands to the second diameter D₂ to accommodate the prosthetic valve. This can cause the braided layer 104 to be foreshortened. However, because the filaments 110 are not engaged or adhered to the layers 102 or 108, the shortening of the braided layer 104 attendant to an increase in the angle θ does not affect the overall length L of the sheath. Moreover, because of the longitudinally-extending folds 126 formed in the layers 102 and 108, the layers 102 and 108 can expand to the second diameter D₂ without rupturing, despite being relatively thin and relatively non-elastic. In this manner, the sheath 100 can resiliently expand from its natural diameter D₁ to a second diameter D₂ that is larger than the diameter D₁ as a prosthetic device is advanced through the sheath, without lengthening and without constricting. Thus, the force required to push the prosthetic implant through the sheath is significantly reduced.

In some examples, because of the radial force applied by the elastic layer 106, the radial expansion of the sheath 100 can be localized to the specific portion of the sheath occupied by the prosthetic device. For example, with reference to FIG. 6, as the prosthetic device 12 moves distally through the sheath 100, the portion of the sheath immediately proximal to the prosthetic device 12 can radially collapse back to the initial diameter D₁ under the influence of the elastic layer 106. The layers 102 and 108 can also buckle as the circumference of the sheath is reduced, causing the ridges 126 and the valleys 128 to reform. This can reduce the size of the sheath required to introduce a prosthetic device of a given size. In some examples, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device and the amount of time for which a given portion of the vessel must be dilated.

In addition to the advantages above, the expandable sheath aspects described herein can provide surprisingly superior performance relative to known introducer sheaths. For example, it is possible to use a sheath configured as described herein to deliver a prosthetic device having a diameter that is two times larger, 2.5 times larger, or even three times larger than the natural outer diameter of the sheath. For example, in one aspect, a crimped prosthetic heart valve having a diameter of 7.2 mm was successfully advanced through a sheath configured as described above and having a natural outer diameter of 3.7 mm. As the prosthetic valve was advanced through the sheath, the outer diameter of the portion of the sheath occupied by the prosthetic valve increased to 8 mm. In other words, it was possible to advance a prosthetic device having a diameter more than two times the outer diameter of the sheath through the sheath, during which the outer diameter of the sheath resiliently increased by 216%. In some examples, a sheath with an initial or natural outer diameter of 4.5 mm to 5 mm can be configured to expand to an outer diameter of 8 mm to 9 mm.

In alternative aspects, the sheath 100 may optionally include layer 102 without layer 108, or layer 108 without layer 102, depending upon the particular characteristics desired.

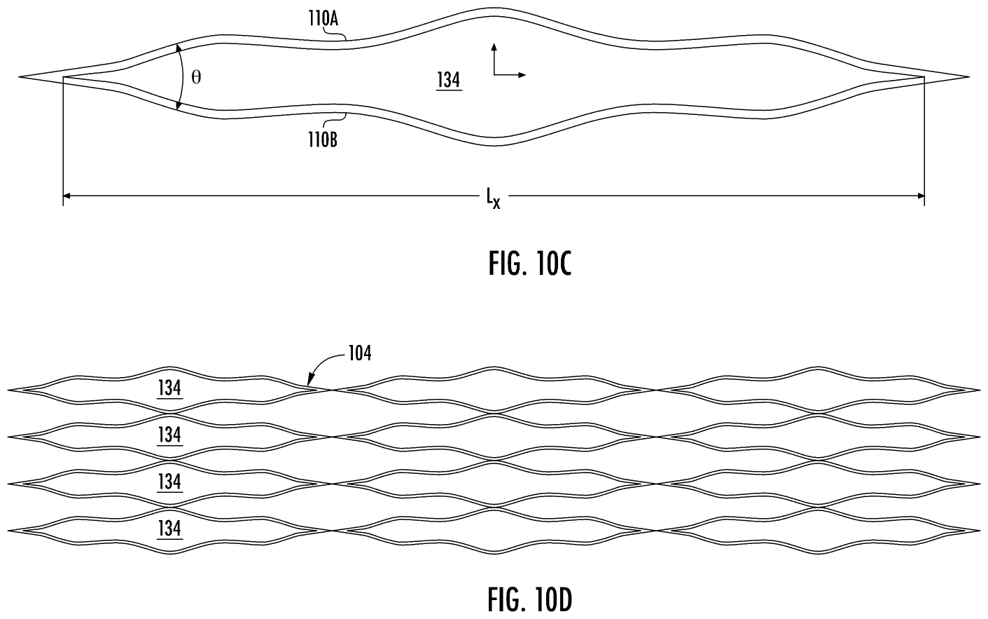

FIGS. 10A-10D illustrate some aspects of the braided layer 104 in which the filaments 110 are configured to buckle. For example, FIG. 10A illustrates a unit cell 134 of the braided layer 104 in a configuration corresponding to the braided layer in a fully expanded state. For example, the expanded state illustrated in FIG. 10A can correspond to the diameter D₂ described above and/or a diameter of the braided layer during the initial construction of the sheath 100 before the sheath is radially collapsed to its functional design diameter D₁, as described below with reference to FIG. 7. The angle θ between the filaments 110A and 110B can be, for example, 40°, and the unit cell 134 can have a length Lₓ along the x-direction (note Cartesian coordinate axes shown). FIG. 10B illustrates a portion of the braided layer 104, including an array of unit cells 134 in the expanded state.

In the illustrated aspects, the braided layer 104 is disposed between the polymeric layers 102 and 108, as described above. For example, the polymeric layers 102 and 108 can be adhered or laminated to each other at the ends of the sheath 100 and/or between the filaments 110 in the open spaces 136 defined by the unit cells 134. Thus, with reference to FIGS. 10C and 10D, when the sheath 100 is radially collapsed to its functional diameter D₁, the diameter of the braided layer 104 can decrease as the angle θ decreases. However, the bonded polymeric layers 102 and 108 can constrain or prevent the braided layer 104 from lengthening as it radially collapses. This can cause the filaments 110 to resiliently buckle in the axial direction, as shown in FIGS. 10C and 10D. The degree of buckling can be such that the length Lₓ of the unit cells 134 is the same, or substantially the same, between the collapsed and fully expanded diameters of the sheath. This means that the overall length of the braided layer 104 can remain constant, or substantially constant, between the natural diameter D₁ of the sheath and the expanded diameter D₂. As the sheath expands from in its initial diameter D₁ during passage of a medical device, the filaments 110 can straighten as the buckling is relieved, and the sheath can radially expand. As the medical device passes through the sheath, the braided layer 104 can be urged back to the initial diameter D₁ by the elastic layer 106, if present, and the filaments 110 can resiliently buckle again. Using the configuration of FIGS. 10A-10C, it is also possible to accommodate a prosthetic device having a diameter that is two times larger, 2.5 times larger, or even three times larger than the natural outer diameter D₁ of the sheath.

Turning now to methods of making expandable sheaths, FIG. 7 illustrates the layers 102-108 of the expandable sheath 100 disposed on a cylindrical mandrel 118, according to one aspect. In certain aspects, the mandrel 118 can have a diameter D₃ that is greater than the desired natural outer diameter D₁ of the finished sheath. For example, in some aspects, a ratio of the diameter D₃ of the mandrel to the outer diameter D₁ of the sheath can be 1.5:1, 2:1, 2.5:1, 3:1, or greater. In certain aspects, the diameter Ds of the mandrel can be equal to the expanded diameter D₂ of the sheath. In other words, the diameter Ds of the mandrel can be the same, or nearly the same, as the desired expanded diameter D₂ of the sheath when a prosthetic device is being advanced through the sheath. Thus, in certain aspects, a ratio of the expanded outer diameter D₂ of the expanded sheath to the collapsed outer diameter D₁ of the unexpanded sheath can be 1.5:1, 2:1, 2.5:1, 3:1, or greater.

With reference to FIG. 7, the expandable sheath 100 can be made by wrapping or situating an ePTFE layer 120 around the mandrel 118, followed by the first polymeric layer 102. In some aspects, the ePTFE layer can aid in removing the sheath 100 from the mandrel 118 upon completion of the fabrication process. The first polymeric layer 102 may be in the form of a pre-fabricated sheet that is applied by being wrapped around the mandrel 118 or may be applied to the mandrel by dip-coating, electro-spinning, etc. The braided layer 104 can be situated around the first layer 102, followed by the elastic layer 106. In aspects where the elastic layer 106 comprises one or more elastic bands 116, the bands 116 can be helically wrapped around the braided layer 104. In some implementations, the elastic layer 106 may be dip-coated, electro-spun, etc. The outer polymeric layer 108 can then be wrapped, situated, or applied around the elastic layer 106, followed by another layer 122 of ePTFE and one or more layers 124 of heat-shrink tubing or heat-shrink tape.

In particular aspects, the elastic bands 116 can be applied to the braided layer 104 in a stretched, taut, or extended condition. For example, in certain aspects, the bands 116 can be applied to the braided layer 104 stretched to a length that is twice their natural, relaxed length. This will cause the completed sheath to radially collapse under the influence of the elastic layer when removed from the mandrel, which can cause corresponding relaxation of the elastic layer, as described below. In some implementations, the layer 102 and the braided layer 104 can be removed from the mandrel, the elastic layer 106 can be applied in a relaxed state or moderately stretched state, and then the assembly can be placed back on the mandrel such that the elastic layer is radially expanded and stretched to a taut condition prior to application of the outer layer 108.

The assembly can then be heated to a sufficiently high temperature that the heat-shrink layer 124 shrinks and compresses the layers 102-108 together. In certain aspects, the assembly can be heated to a sufficiently high temperature such that the polymeric inner and outer layers 102 and 108 become soft and tacky and bond to each other in the open spaces between the braided layer 104 and the elastic layer 106 and encapsulate the braided layer and the elastic layer. In some implementations, the inner and outer layers 102, 108 can be reflowed or melted such that they flow around and through the braided layer 104 and the elastic layer 106. In an exemplary aspect, the assembly can be heated at 150° C. for 20-30 minutes.

After heating, the sheath 100 can be removed from the mandrel 118, and the heat-shrink tubing 124 and the ePTFE layers 120 and 122 can be removed. In such exemplary aspects, these ePTFE layers can behave as sacrificial layers. Upon being removed from the mandrel 118, the sheath 100 can at least partially radially collapse to the natural design diameter D₁ under the influence of the elastic layer 106. In certain aspects, the sheath can be radially collapsed to the design diameter with the optional aid of a crimping mechanism. The attendant reduction in circumference can buckle the filaments 110, as shown in FIGS. 10C and 10D, along with the inner and outer layers 102 and 108 to form the longitudinally-extending folds 126.

In certain aspects, a layer of PTFE can be interposed between the ePTFE layer 120 and the inner layer 102, and/or between the outer layer 108 and the ePTFE layer 122, in order to facilitate separation of the inner and outer polymeric layers 102, 108 from the respective ePTFE layers 120 and 122. In some implementations, one of the inner layer 102 or the outer layer 108 may be omitted, as described above.

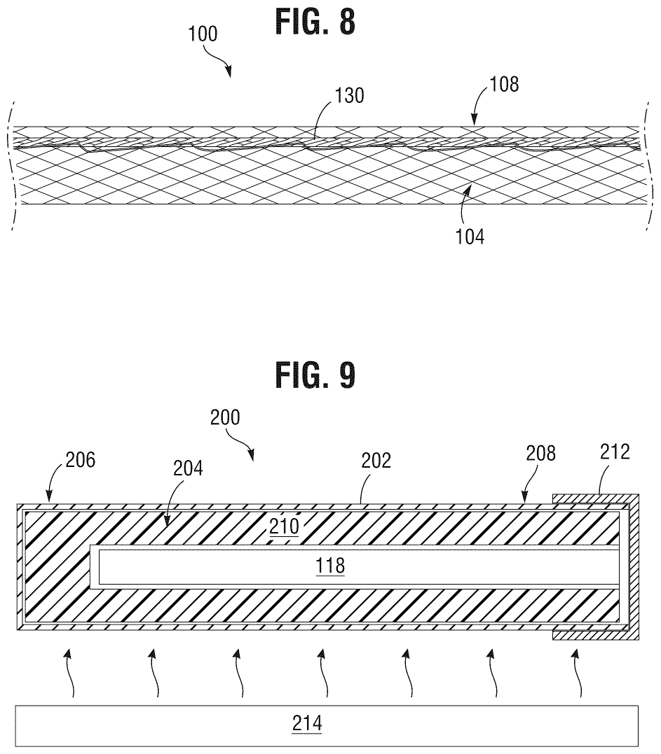

FIG. 8 illustrates some aspects of the expandable sheath 100, including one or more members configured as yarns or cords 130 extending longitudinally along the sheath and attached to the braided layer 104. Although only one cord 130 is illustrated in FIG. 8, in practice, the sheath may include two cords, four cords, six cords, etc., arrayed around the circumference of the sheath at equal angular spacings. The cords 130 can be sutured to the exterior of the braided layer 104, although other configurations and attachment methods are possible. By virtue of being attached to the braided layer 104, the cords 130 can be configured to prevent axial elongation of the braided layer 104 when a prosthetic device is passed through the sheath. The cords 130 may be employed in combination with the elastic layer 106 or separately. The cords 130 may also be used in combination with one or both of the inner and/or outer layers 102 and 108, depending upon the particular characteristics desired. The cords 130 may also be disposed on the inside of the braided layer 104 (e.g., between the inner layer 102 and the braided layer 104).

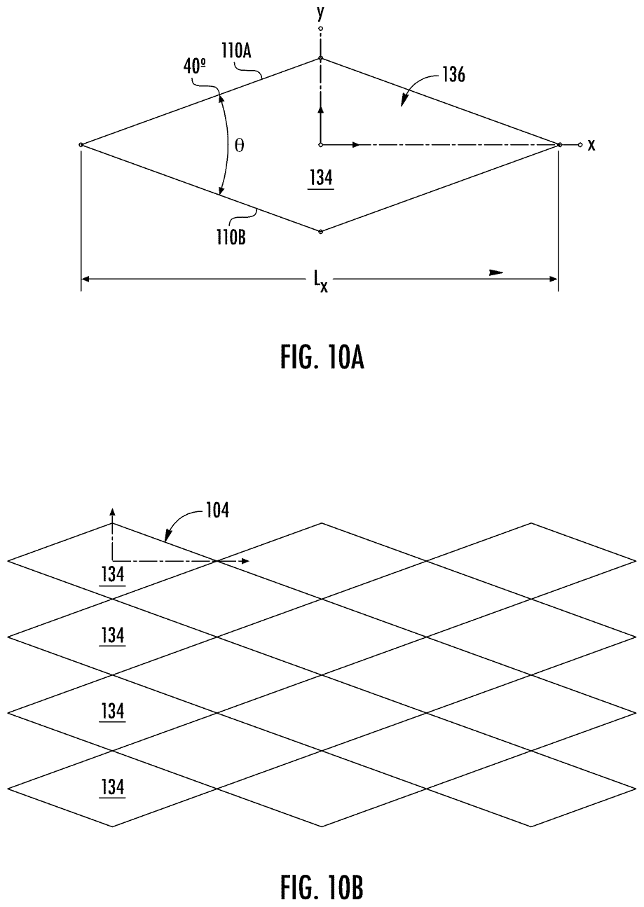

The expandable sheath 100 can also be made in other ways. For example, FIG. 9 illustrates an apparatus 200, including a containment vessel 202 and a heating system schematically illustrated at 214. The apparatus 200 is particularly suited for forming devices (medical devices or devices for non-medical uses) comprised of two or more layers of material. Devices formed by the apparatus 200 can be formed from two or more co-axial layers of material, such as the sheath 100 or shafts for catheters. Devices formed by the apparatus 200 alternatively can be formed by two or more non-coaxial layers, such as two or more layers stacked on top of each other.

The containment vessel 202 can define an interior volume or chamber 204. In the illustrated aspect, vessel 202 can be a metal tube, including a closed-end 206 and an open-end 208. The vessel 202 can be at least partially filled with a thermally-expandable material 210 having a relatively high coefficient of thermal expansion. In particular aspects, the thermally-expandable material 210 may have a coefficient of thermal expansion of 2.4×10⁻⁴/° C. or greater. Exemplary thermally-expandable materials include elastomers, such as silicone materials. Silicone materials can have a coefficient of thermal expansion of from 5.9×10⁻⁴/° C. to 7.9×10⁻⁴/° C.

A mandrel, similar to the mandrel 118 of FIG. 7 and including the desired combination of sheath material layers disposed around, can be inserted into the thermally-expandable material 210. Alternatively, mandrel 118 can be inserted into chamber 204, and the remaining volume of the chamber can be filled with the thermally-expandable material 210 so that the mandrel is surrounded by the material 210. The mandrel 118 is shown schematically for purposes of illustration. As such, mandrel 118 can be cylindrical, as depicted in FIG. 7. Likewise, the inner surface of material 210 and the inner surface of the vessel 202 can have a cylindrical shape corresponding to the shape of the mandrel 118 and the final shape of the sheath 100. To facilitate the placement of a cylindrical or rounded mandrel 118, vessel 202 can comprise two portions that are connected to each other by a hinge to allow the two portions to move between an open configuration for placing the mandrel inside of the vessel and a closed configuration extending around the mandrel. For example, the upper and lower halves of the vessel shown in FIG. 9 can be connected to each other by a hinge at the closed side of the vessel (the left side of the vessel in FIG. 9).

The open end 208 of vessel 202 can be closed with a cap 212. The vessel 202 can then be heated by the heating system 214. Heating by the heating system 214 can cause the material 210 to expand within chamber 204 and apply radial pressure against the layers of material on the mandrel 118. The combination of the heat and pressure can cause the layers on the mandrel 118 to bond or adhere to each other to form a sheath. In certain aspects, it is possible to apply radial pressure of 100 MPa or more to the mandrel 118 using apparatus 200. The amount of radial force applied to the mandrel can be controlled by, for example, the type and quantity of the material 210 selected and its coefficient of thermal expansion, the thickness of the material 210 surrounding the mandrel 118, the temperature to which the material 210 is heated, etc.

In some aspects, the heating system 214 can be an oven into which the vessel 202 is placed. In some aspects, the heating system can include one or more heating elements positioned around vessel 202. In some aspects, vessel 202 can be an electrical resistance heating element or an induction heating element controlled by the heating system 214. In some aspects, heating elements can be embedded in the thermally-expandable material 210. In some aspects, the material 210 can be configured as a heating element by, for example, adding electrically conductive filler materials, such as carbon fibers or metal particles.

The apparatus 200 can provide several advantages over known methods of sheath fabrication, including uniform, highly controllable application of radial force to the mandrel 118 along its length and high repeatability. The apparatus 200 can also facilitate fast and accurate heating of the thermally-expandable material 210 and can reduce or eliminate the need for heat-shrink tubing and/or tape, reducing material costs and labor. The amount of radial force applied can also be varied along the length of the mandrel by, for example, varying the type or thickness of the surrounding material 210. In certain aspects, multiple vessels 202 can be processed in a single fixture, and/or multiple sheaths can be processed within a single vessel 202. The apparatus 200 can also be used to produce other devices, such as shafts or catheters.

In one specific method, the sheath 100 can be formed by placing layers 102, 104, 106, 108 on the mandrel 118 and placing the mandrel with the layers inside of the vessel 202 with the thermally-expandable material 210 surrounding the outermost layer 108. If desired, one or more inner layers 120 of ePTFE (or similar material) and one or more outer layers 122 of ePTFE (or similar material) can be used (as shown in FIG. 7) to facilitate the removal of the finished sheath from the mandrel 118 and the material 210. The assembly is then heated with the heating system 214 to reflow layers 102, 108. Upon subsequent cooling, layers 102, 108 become at least partially bonded to each other and at least partially encapsulate layers 104, 106.

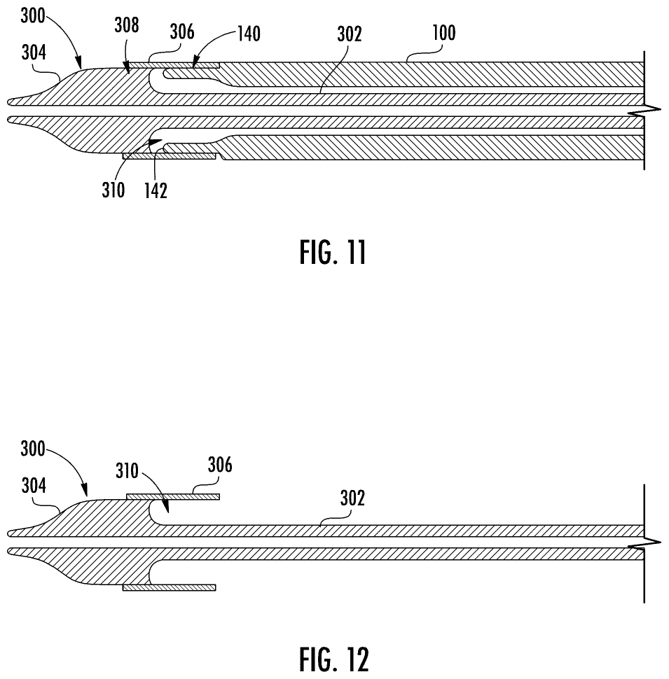

FIG. 11 illustrates some aspects in which the expandable sheath 100 is configured to receive an apparatus configured as a pre-introducer or vessel dilator 300. In particular aspects, the introducer device 90 can include the vessel dilator 300. Referring to FIG. 12, the vessel dilator 300 can comprise a shaft member 302, including a tapered dilator member configured as a nose cone 304 located at the distal end portion of the shaft member 302. The vessel dilator 300 can comprise a capsule or retaining member 306 extending proximally from a proximal end portion 308 of the nose cone 304 such that a circumferential space 310 is defined between the exterior surface of the shaft member 302 and the interior surface of the retaining member 306. In certain aspects, the retaining member 306 can be configured as a thin polymeric layer or sheet, as described below.

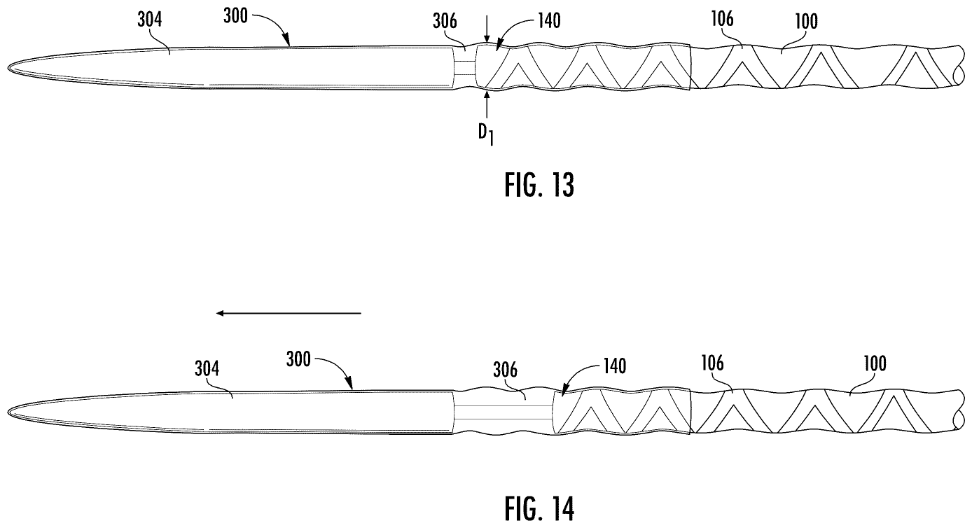

Referring to FIGS. 11 and 13, a first or distal end portion 140 of the sheath 100 can be received in space 310 such that the sheath engages the nose cone 304 and/or such that the retaining member 306 extends over the distal end portion 140 of the sheath. In use, the coupled or assembled vessel dilator 300 and sheath 100 can then be inserted through an incision into a blood vessel. The tapered cone shape of the nose cone 304 can aid in gradually dilating the blood vessel and access site while minimizing trauma to the blood vessel and surrounding tissue. Once the assembly has been inserted to the desired depth, the vessel dilator 300 can be advanced further into the blood vessel (e.g., distally) while the sheath 100 is held steady, as illustrated in FIG. 14.

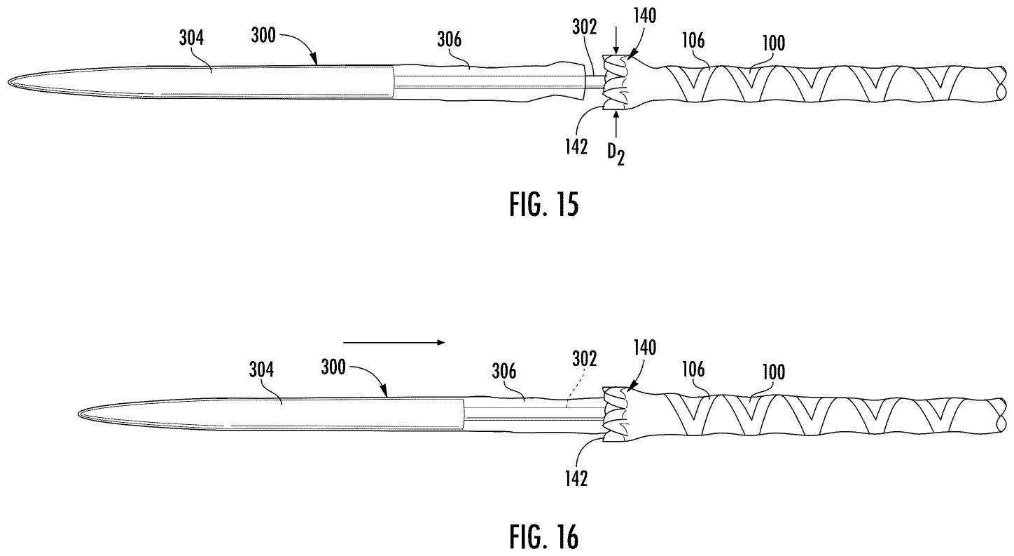

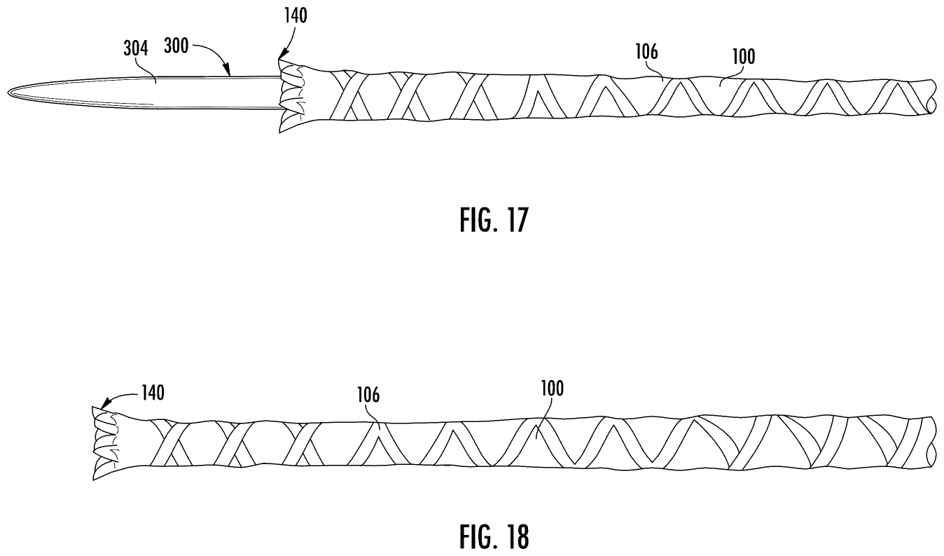

Referring to FIG. 15, the vessel dilator 300 can be advanced distally through the sheath 100 until the retaining member 306 is removed from over the distal end portion 140 of the sheath 100. In certain aspects, the helically-wrapped elastic layer 106 of the sheath can terminate proximally of the distal end 142 of the sheath. Thus, when the distal end portion 140 of the sheath is uncovered, the distal end portion (which can be heat-set) can flare or expand, increasing the diameter of the opening at the distal end 142 from the first diameter D₁ (FIG. 13) to a second, larger diameter D₂ (FIG. 15). The vessel dilator 300 can then be withdrawn through the sheath 100, as illustrated in FIGS. 16-18, leaving the sheath 100 in place in the vessel.

The vessel dilator 300 can include a variety of active and/or passive mechanisms for engaging and retaining the sheath 100. For example, in certain aspects, the retaining member 306 can comprise a polymeric heat-shrink layer that can be collapsed around the distal end portion of the sheath 100. In the aspect illustrated in FIG. 1, the retaining member can comprise an elastic member configured to compress the distal end portion 140 of the sheath 100. In some aspects, the retaining member 306 and the sheath 100 can be glued or fused (e.g., heat-bonded) together in a manner such that application of a selected amount of force can break the adhesive bonds between retaining member 306 free from the sheath 100 to allow the vessel dilator to be withdrawn. In some aspects, the end portion of the braided layer 104 can be heat set to flare or expand radially inwardly or outwardly in order to apply pressure to a corresponding portion of the vessel dilator 300.

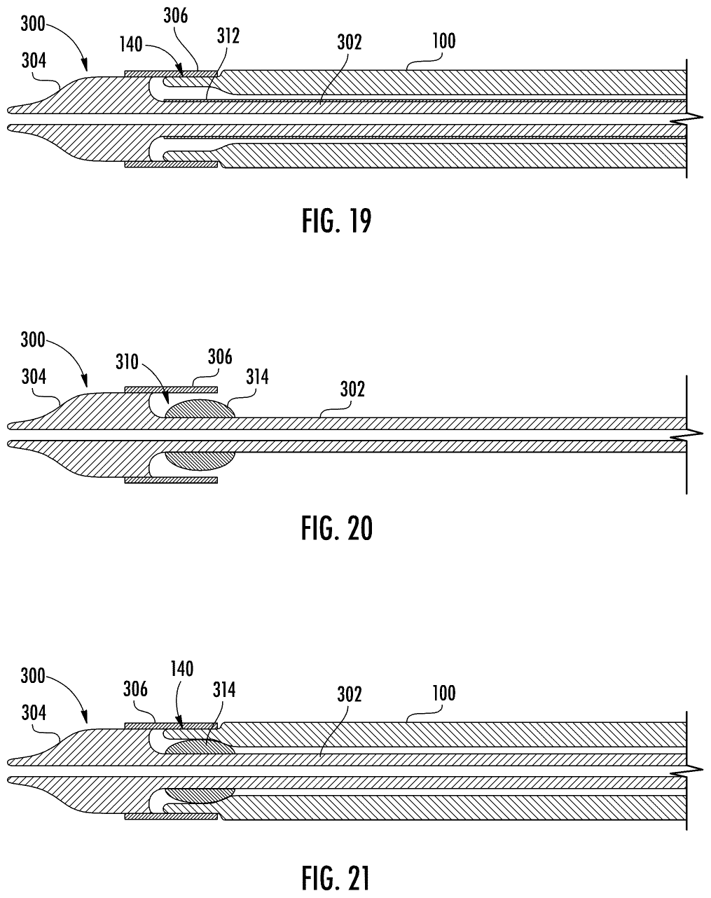

Referring to FIG. 19, the assembly can include a mechanically-actuated retaining mechanism, such as a shaft 312 disposed between the dilator shaft member 302 and the sheath 100. In certain aspects, shaft 312 can releasably couple the vessel dilator 300 to the sheath 100 and can be actuated from outside the body (i.e., manually deactivated).

Referring to FIGS. 20 and 21, in some aspects, the shaft 304 can comprise one or more balloons 314 arrayed circumferentially around its exterior surface and configured to engage the sheath 100 when inflated. The balloons 314 can be selectively deflated in order to release the sheath 100 and withdraw the vessel dilator. For example, when inflated, the balloons press the captured distal end portion of the sheath 100 against the inner surface of capsule 306 to assist in retaining the sheath in place relative to the vessel dilator. When the balloons are deflated, the vessel dilator can be more easily moved relative to the sheath 100.

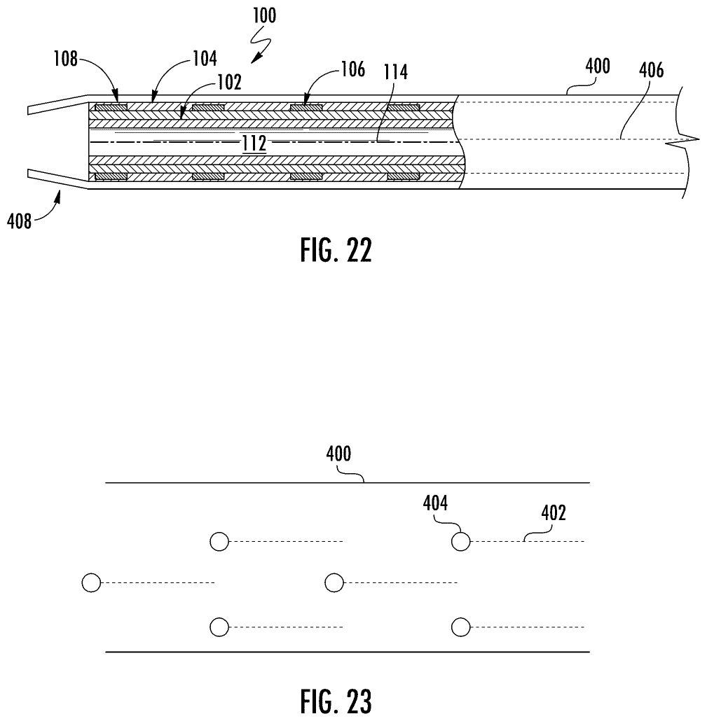

In some aspects, an expandable sheath configured as described above can further comprise a shrinkable polymeric outer cover, such as a heat-shrink tubing layer 400 shown in FIG. 22. The heat-shrink tubing layer 400 can be configured to allow a smooth transition between the vessel dilator 300 and the distal end portion 140 of the sheath. The heat-shrink tubing layer 400 can also constrain the sheath to a selected initial, reduced outer diameter. In certain aspects, the heat-shrink tubing layer 400 extends fully over the length of the sheath 100 and can be attached to the sheath handle by a mechanical fixation means, such as a clamp, nut, adhesive, heat welding, laser welding, or an elastic clamp. In some aspects, the sheath is press-fit into the heat-shrink tubing layer during manufacturing.

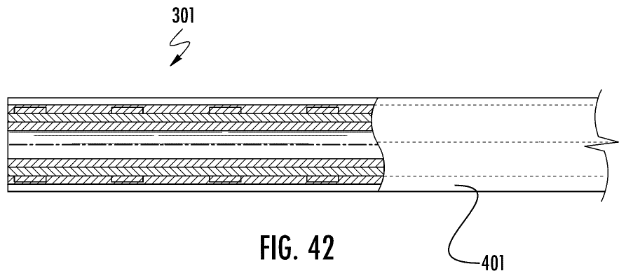

In some aspects, the heat-shrink tubing layer 400 can extend distally beyond the distal end portion 140 of the sheath as the distal overhang 408 is shown in FIG. 22. A vessel dilator can be inserted through the sheath lumen 112 and beyond the distal edge of the overhang 408. The overhang 408 conforms tightly to the inserted vessel dilator to give a smooth transition between the dilator diameter and the sheath diameter to ease the insertion of the combined dilator and sheath. When the vessel dilator is removed, overhang 408 remains in the vessel as part of sheath 100. The heat shrink tubing layer 400 offers the additional benefit of shrinking the overall outer diameter of the sheath along the longitudinal axis. However, it will be understood that some aspects, such as sheath 301 shown in FIG. 42 may have a heat-shrink tubing layer 401 that stops at the distal end of the sheath 301 or, in some aspects, does not extend fully to the distal end of the sheath. In aspects without distal overhangs, the heat-shrink tubing layer functions mainly as an outer shrinking layer, configured to maintain the sheath in a compressed configuration. Such aspects will not result in a flapping overhang at the distal end of the sheath once the dilator is retrieved.

In some aspects, the heat-shrink tubing layer can be configured to split open as a delivery apparatus, such as the delivery apparatus 10 is advanced through the sheath. For example, in certain aspects, the heat-shrink tubing layer can comprise one or more longitudinally extending openings, slits, or weakened, elongated scorelines 406, such as those shown in FIG. 22 configured to initiate splitting of the layer at a selected location. As the delivery apparatus 10 is advanced through the sheath, the heat-shrink tubing layer 400 can continue to split open, allowing the sheath to expand as described above with reduced force. In certain aspects, the sheath need not comprise the elastic layer 106 such that the sheath automatically expands from the initial, reducing the diameter when the heat-shrink tubing layer splits open. The heat shrink tubing layer 400 can comprise polyethylene or other suitable materials.

FIG. 23 illustrates a heat-shrink tubing layer 400 that can be placed around the expandable sheaths described herein, according to one aspect. In some aspects, the heat-shrink tubing layer 400 can comprise a plurality of cuts or scorelines 402 extending axially along the tubing layer 400 and terminating at distal stress relief features configured as circular openings 404. It is contemplated that the distal stress relief feature can be configured as any other regular or irregular curvilinear shape, including, for example, oval and/or ovoid-shaped openings. It is also contemplated various-shaped distal stress relief features along and around the heat-shrink tubing layer 400. As the delivery apparatus 10 is advanced through the sheath, the heat-shrink tubing layer 400 can split open along the scorelines 402, and the distally positioned openings 404 can arrest further tearing or splitting of the tubing layer along the respective scorelines. As such, the heat-shrink tubing layer 400 remains attached to the sheath along the sheath length. In the illustrated aspect, the scorelines and associated openings 404 are longitudinally and circumferentially offset from one another or staggered. Thus, as the sheath expands, the scorelines 402 can form rhomboid structures. The scorelines can also extend in other directions, such as helically around the longitudinal axis of the sheath or in a zig-zag pattern.

In some aspects, splitting or tearing of the heat-shrink tubing layer may be induced in a variety of other ways, such as by forming weakened areas on the tubing surface by, for example, applying chemical solvents, cutting, scoring, or ablating the surface with an instrument or laser, and/or by decreasing the wall thickness or making cavities in the tubing wall (e.g., by femtosecond laser ablation).

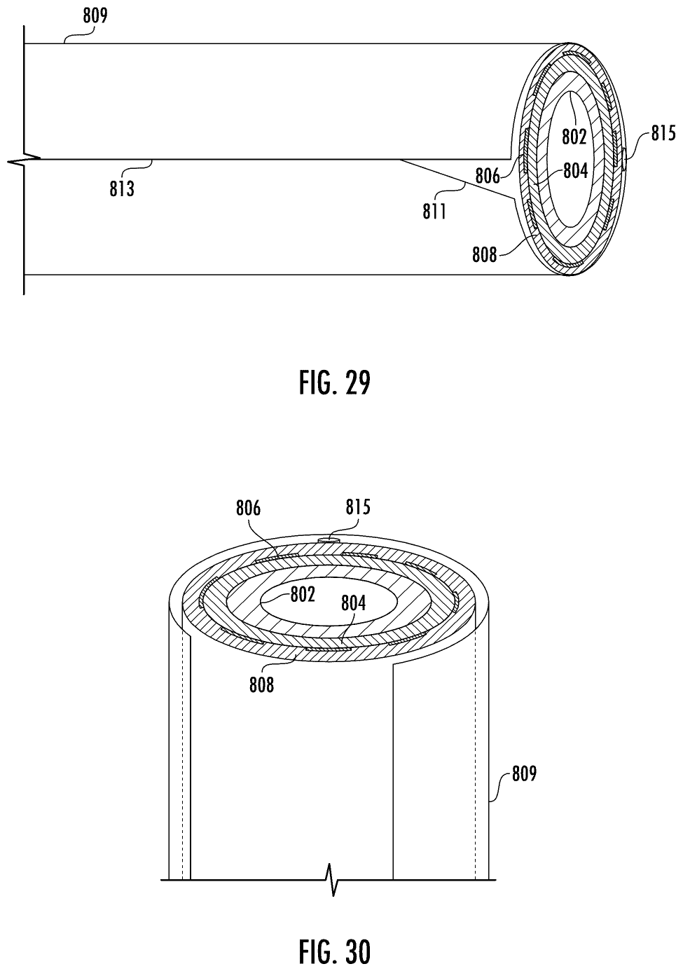

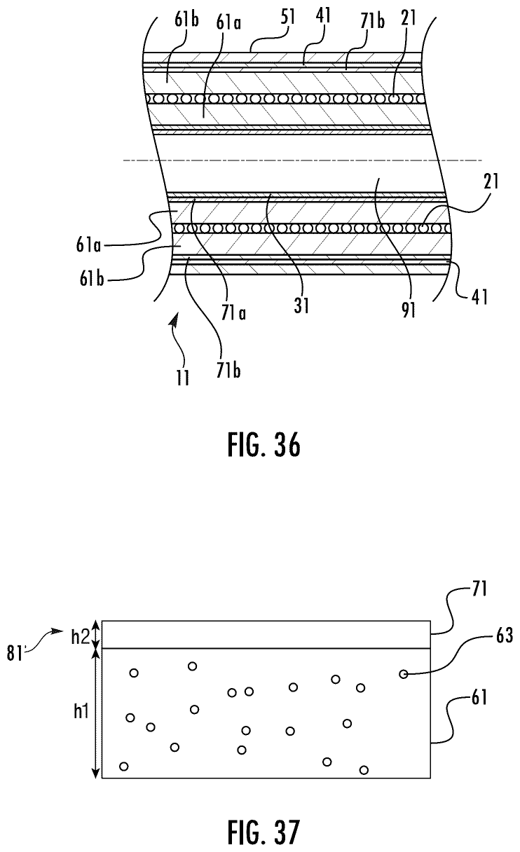

In some aspects, the heat-shrink tubing layer may be attached to the body of the sheath by adhesive, welding, or any other suitable fixation means. FIG. 29 shows a perspective view of a sheath aspect, including an inner layer 802, a braided layer 804, an elastic layer 806, an outer layer 808, and a heat shrink tubing layer 809. As described below, with respect to FIG. 36, some aspects do not include elastic layer 806. Heat shrink tubing layer 809 includes a split 811 and a perforation 813 extending along the heat shrink tubing layer 809. Heat shrink tubing layer 809 is bonded to the outer layer 808 at an adhesive seam 815. For example, in certain aspects, the heat-shrink tubing layer 809 can be welded, heat-bonded, chemically bonded, ultrasonically bonded, and/or bonded using adhesive agents (including, but not limited to, hot glue, for example, LDPE fiber hot glue) at seam 815. The outer layer 808 can be bonded to the heat shrink tubing layer 809 axially along the sheath at a seam 815 or in a spiral or helical fashion. FIG. 30 shows the same sheath aspect with heat shrink tubing layer 809 split open at the distal end of the sheath.

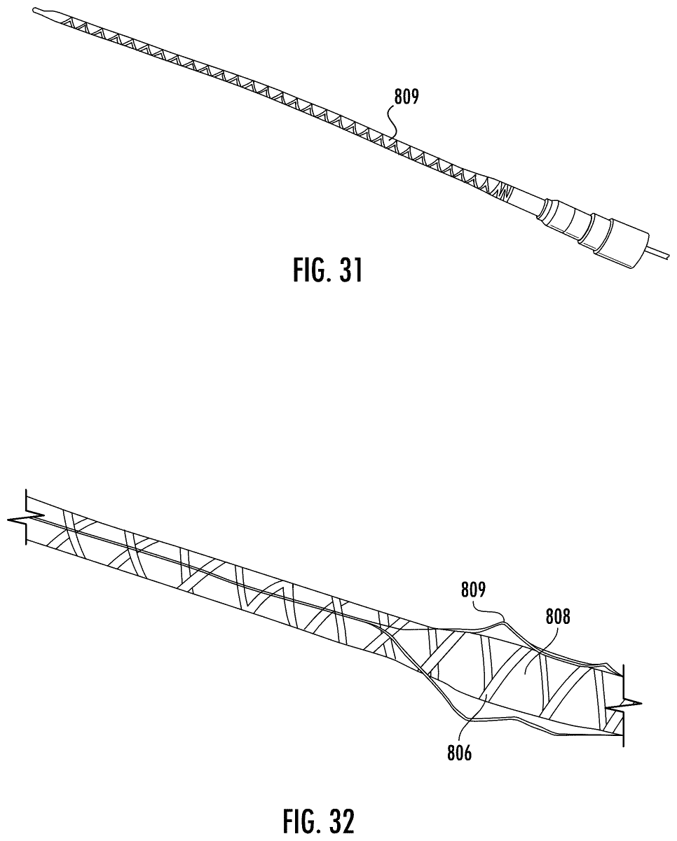

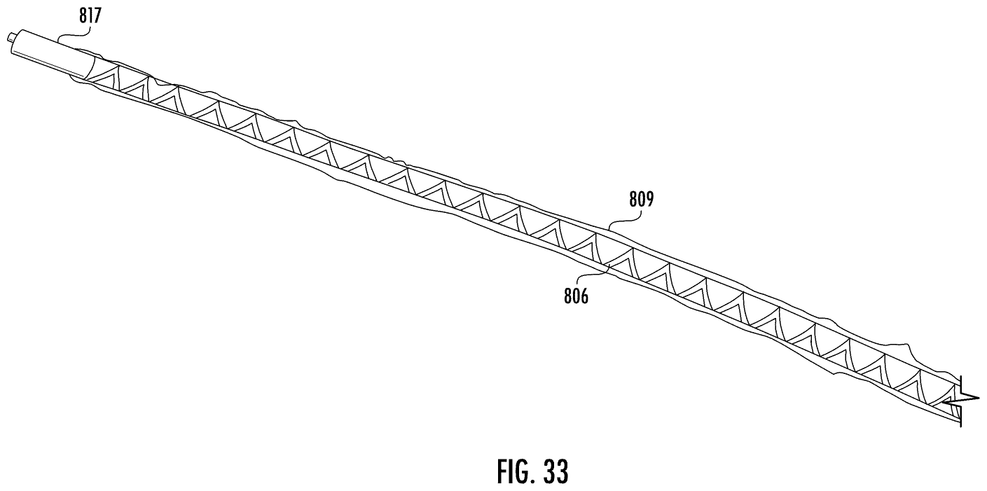

FIG. 31 shows a sheath having a heat shrink tubing layer 809, but prior to movement of a delivery system, therethrough. FIG. 32 shows a perspective view of a sheath wherein the heat shrink tubing layer 809 has been partially torn open and detached as a passing delivery system widens the diameter of the sheath. Heat shrink tubing layer 809 is being retained by the adhesive seam 815. Attaching the heat-shrink tubing layer 809 to the sheath in this manner can help to keep the heat-shrink tubing layer 809 attached to the sheath after the layer splits and the sheath has expanded, as shown in FIG. 33, where delivery system 817 has moved completely through the sheath and torn the heat shrink tubing layer 809 along the entire length of the sheath.

In some aspects, the expandable sheath can have a distal end or tip portion comprising an elastic thermoplastic material (e.g., Pebax), which can be configured to provide an interference fit or interference geometry with the corresponding portion of the vessel dilator 300. In certain configurations, the outer layer of the sheath may comprise polyamide (e.g., nylon) in order to provide for welding the distal end portion to the body of the sheath. In certain aspects, the distal end portion can comprise a deliberately weakened portion, scoreline, slit, etc., to allow the distal end portion to split apart as the delivery apparatus is advanced through the distal end portion.

In some implementations, the entire sheath could have an elastomeric outer cover that extends longitudinally from the handle to the distal end portion 140 of the sheath, optionally extending onward to create an overhang similar to overhang 408 shown in FIG. 22. The elastomeric overhang portion conforms tightly to the vessel dilator but remains a part of the sheath once the vessel dilator is removed. As a delivery system is passed through, the elastomeric overhang portion expands and then collapses to allow it to pass. The elastomeric overhang portion, or the entire elastomeric outer cover, can include deliberately weakened portions, scorelines, slits, etc., to allow the distal end portion to split apart as the delivery apparatus is advanced through the distal end portion.

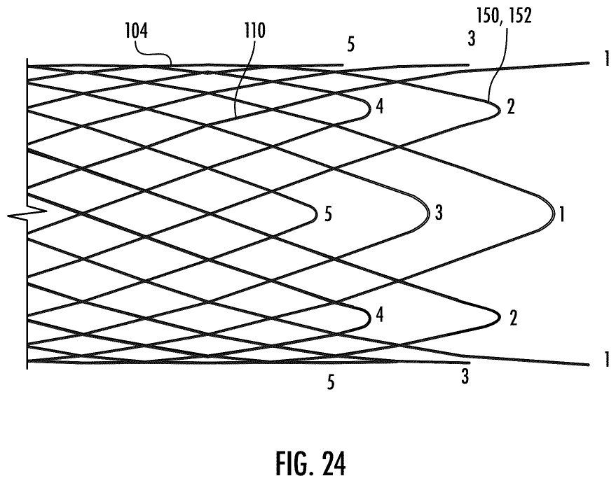

FIG. 24 illustrates an end portion (e.g., a distal end portion) of some aspects of the braided layer 104 in which portions 150 of the braided filaments 110 are bent to form loops 152, such that the filaments loop or extend back in the opposite direction along the sheath. The filaments 110 can be arranged such that the loops 152 of various filaments 110 are axially offset from each other in the braid. Moving toward the distal end of the braided layer 104 (to the right in the figure), the number of braided filaments 110 can decrease. For example, the filaments indicated at 5 can form loops 152 first, followed by the filaments indicated at 4, 3, and 2, with the filaments at 1 forming the distal-most loops 152. Thus, the number of filaments 110 in the braid decreases in the distal direction, which can increase the radial flexibility of the braided layer 104.

In some aspects, the distal end portion of the expandable sheath can comprise a polymer such as Dyneema®, which can be tapered to the diameter of the vessel dilator 300. Weakened portions, such as dashed cuts, scoring, etc., can be applied to the distal end portion such that it will split open and/or expand in a repeatable way. In some implementations and as also described in detail below, the distal end portion of the expandable sheath can comprise any of the disclosed herein layers.

Crimping of the expandable sheath aspects described herein can be performed in a variety of ways, as described above. According to some aspects, the sheath can be crimped using a conventional short crimper several times longitudinally along the longer sheath. In some aspects, the sheath may be collapsed to a specified crimped diameter in one or a series of stages in which the sheath is wrapped in heat-shrink tubing and collapsed under heating. For example, a first heat shrink tube can be applied to the outer surface of the sheath, the sheath can be compressed to an intermediate diameter by shrinking the first heat shrink tube (via heat), the first heat shrink tube can be removed, a second heat shrink tube can be applied to the outer surface of the sheath, the second heat shrink tube can be compressed via heat to a diameter smaller than the intermediate diameter, and the second heat shrink tube can be removed. This can go on for as many rounds as necessary to achieve the desired crimped sheath diameter.

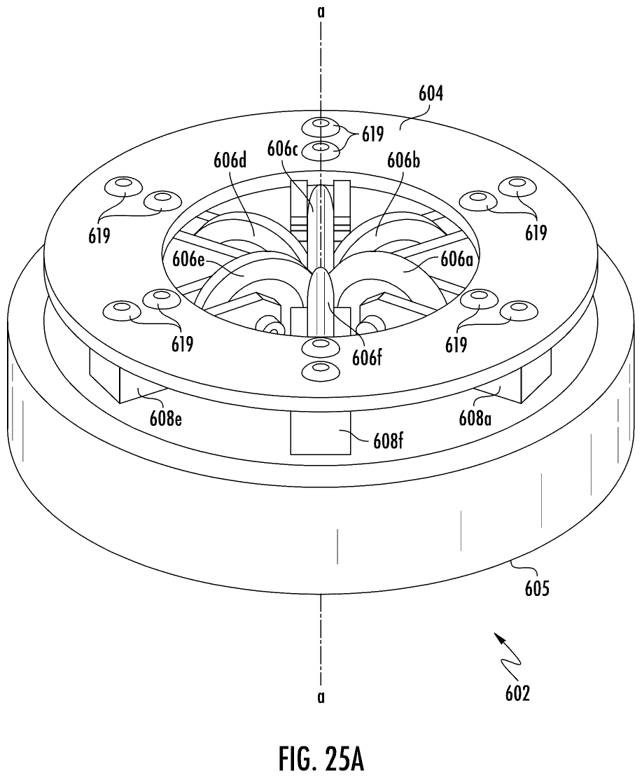

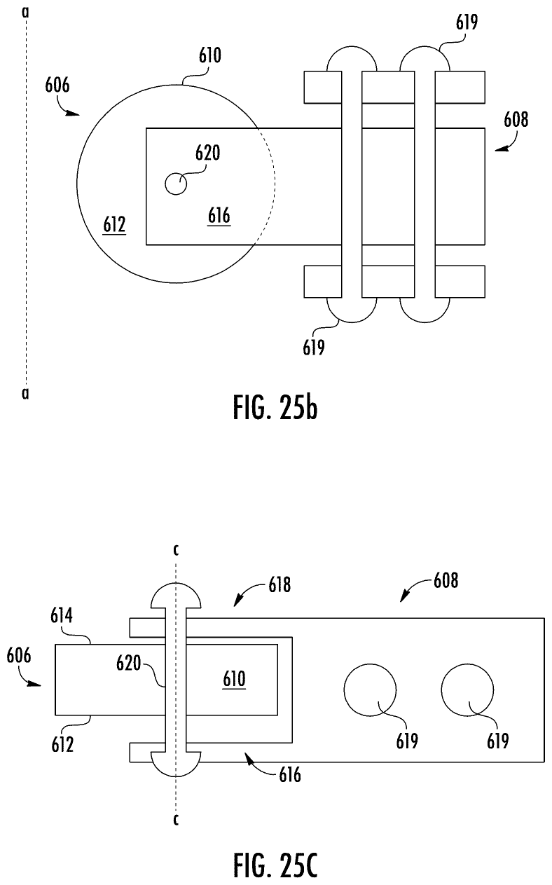

Crimping of the expandable sheath aspects described herein can be performed in a variety of ways, as described above. A roller-based crimping mechanism 602, such as the one shown in FIGS. 25A-25C can be advantageous for crimping elongated structures such as the sheaths disclosed herein. The crimping mechanism 602 has a first end surface 604, a second end surface 605, and a longitudinal axis a-a extending between the first and second end surfaces 604, 605. A plurality of disc-shaped rollers 606a-f are radially arranged about the longitudinal axis a-a, each positioned at least partially between the first and second end surfaces of the crimping mechanism 602. Six rollers are depicted in the aspect shown, but the number of rollers may vary. Each disc-shaped roller 606 is attached to the larger crimping mechanism by a connector 608. A side cross-sectional view of an individual disc-shaped roller 606 and connector 608 is shown in FIG. 25B and a top view of an individual disc-shaped roller 606 and connector 608 is shown in FIG. 25C. An individual disc-shaped roller 606 has a circular edge 610, a first side surface 612, a second side surface 614, and a central axis c-c extending between center points of first and second side surfaces 612, 614, as shown in FIG. 25C. The plurality of disc-shaped rollers 606a-f are radially arranged about the longitudinal axis a-a of the crimping mechanism 602 such that each central axis o-c of a disc-shaped roller 606 is oriented perpendicularly to the longitudinal axis a-a of the crimping mechanism 602. The circular edges 610 of the disc-shaped rollers partially define a passage that extends axially through the crimping mechanism 602 along longitudinal axis a-a.

Each disc-shaped roller 606 is held in place in the radially arranged configuration by a connector 608 that is attached to crimping mechanism 602 via one or more fasteners 619, such that the location of each of the plurality of connectors is fixed with respect to the first end surface of the crimping mechanism 602. In the depicted aspect, fasteners 619 are positioned adjacent an outer portion of the crimping mechanism 602, radially outwardly of the disc-shaped rollers 606. Two fasteners 619 are used to position each connector 608, in the aspect shown, but the number of fasteners 619 can vary. As shown in FIGS. 25B and 25C, a connector 608 has a first arm 616 and a second arm 618. First and second arms 616, 618 extend over a disc-shaped roller 608 from a radially-outward portion of circular edge 610 to a central portion of the disc-shaped roller 608. A bolt 620 extends through the first and second arms 616, 618 and through a central lumen of the disc-shaped roller 608, the central lumen passing from a center point of front surface 612 to a center point of the back surface 614 of the disc-shaped roller 606 along central axis c-C. The bolt 620 is positioned loosely within the lumen, with substantial clearance/space to allow the disc-shaped roller 608 to rotate about central axis c-C.

During use, an elongated sheath is advanced from the first side 604 of the crimping mechanism 602 through the axial passage between the rollers and out the second side 605 of the crimping mechanism 602. The pressure from the circular edge 610 of the disc-shaped rollers 606 reduces the diameter of the sheath to a crimped diameter as it rolls along the outer surface of the elongated sheath.

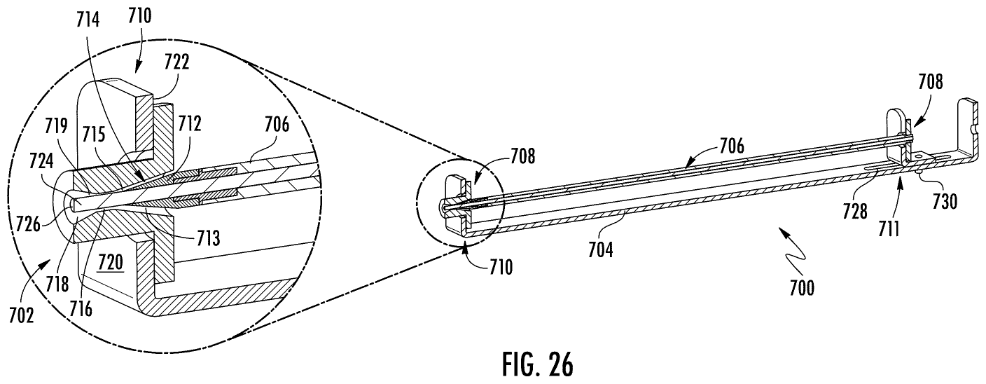

FIG. 26 shows an aspect of a crimping device 700 designed to facilitate the crimping of elongated structures, such as sheaths. The crimping device includes an elongated base 704, an elongated mandrel 706 positioned above the elongated base 704, and a holding mechanism 708 attached to the elongated base 704. The holding mechanism 708 supports the mandrel 706 in an elevated position above base 704. The holding mechanism includes a first end piece 710 that includes a crimping mechanism 702. The mandrel 706 includes a conical end portion 712 that nests within a first tapered portion 713 of a narrowing lumen 714 of the first end piece 710. The conical end portion 712 of mandrel 706 is positioned loosely within the narrowing lumen 714, with enough space or clearance between the conical end portion 712 and the lumen 714 to allow for passage of an elongated sheath over the conical end portion 712 of mandrel 706 and through the narrowing lumen 714. During use, the conical end portion 712 helps to avoid circumferential buckling of the sheath during crimping. In some aspects, the mandrel 706 can also include a cylindrical end portion 724 that extends outwardly from the conical end portion 712 and defines an end 726 of the mandrel 706.

The first tapered portion 713 of the narrowing lumen 714 opens toward a second end piece 711 of the holding mechanism 708, such that the widest side of the taper is located on an inner surface 722 of the first end piece 710. In the aspect shown, the first tapered portion 713 narrows to a narrow end 715 that connects with a narrow cylindrical portion 716 of the narrowing lumen 714. In this aspect, the narrow cylindrical portion 716 defines the narrowest diameter of the narrowing lumen 714. The cylindrical end portion 724 of the mandrel 706 may nest loosely within the narrow cylindrical portion 716 of the narrowing lumen 714, with enough space or clearance between the cylindrical end portion 724 and the narrow cylindrical portion 716 of the lumen to allow for passage of the elongated sheath. The elongated nature of the narrow cylindrical portion 716 may facilitate the smoothing of the crimped sheath after it has passed over the conical end portion 712 of the mandrel. However, the length of the cylindrical portion 716 of the narrowing lumen 714 is not meant to limit the disclosure, and in some aspects, the crimping mechanism 702 may only include a first tapered portion 713 of the narrowing lumen 714 and still be effective to crimp an elongated sheath.

At the opposite end of the first end piece, 710, shown in FIG. 26, a second tapered portion 718 of the narrowing lumen 714 opens up from narrow cylindrical portion 716 such that the widest side of the taper is located on the outer surface 720 of the first end piece 710. The narrow end 719 of the second tapered portion 718 connects with the narrow cylindrical portion 716 of the narrowing lumen 714 in the interior of the crimping mechanism 702. The second tapered portion 718 of the narrowing lumen 714 may not be present in some aspects.

The holding mechanism 708 further includes a second end piece 711 positioned opposite the elongated base 704 from the first end piece 710. The second end piece 711 is movable with respect to elongated base 704, such that the distance between the first end piece 710 and the second end piece 711 is adjustable and, therefore, able to support mandrels of varying sizes. In some aspects, elongated base 704 may include one or more elongated sliding tracks 728. The second end piece 711 can be slidably engaged to the sliding track 728 via at least one reversible fastener 730, such as, but not limited to, a bolt that extends into or through the second end piece 711 and the elongated sliding track 728. To move the second end piece 711, the user would loosen or remove the reversible fastener 730, slide the second end piece 711 to the desired location, and replace or tighten the reversible fastener 730.

In use, a sheath in an uncrimped diameter can be placed over the elongated mandrel 706 of the crimping device 700, shown in FIG. 26, such that the inner surface of the entire length of the uncrimped sheath is supported by the mandrel. The uncrimped sheath is then advanced over the conical end portion 712 and through the narrowing lumen 714 of the crimping mechanism 702. The uncrimped sheath is crimped to a smaller, crimped diameter via pressure from the interior surface of the narrowing lumen 714. In some aspects, the sheath is advanced through both a first tapered portion 713 and a cylindrical portion 716 of the narrowing lumen 714 before exiting the crimping mechanism 702. In some aspects, the sheath is advanced through a first tapered portion 713, a cylindrical portion 716, and a second tapering portion 718 of the narrowing lumen 714 before exiting the crimping mechanism 702.

In some aspects, the crimping mechanism 602 shown in FIG. 25A may be positioned within a larger crimping device, such as crimping device 700, shown in FIG. 26. For example, the crimping mechanism 602 can be positioned within the first end piece 710 of crimping device 700 instead of, or in combination with, crimping mechanism 702. For example, the rolling crimping mechanism 602 could entirely replace the narrowing lumen 714 of crimping mechanism 702, or the rolling crimping mechanism 602 could be nested within the narrow cylindrical portion 716 of the narrowing lumen 714 of the crimping mechanism 702, such that the first tapered portion 713 feeds the expandable sheath through the plurality of radially arranged disc-shaped rollers 606.

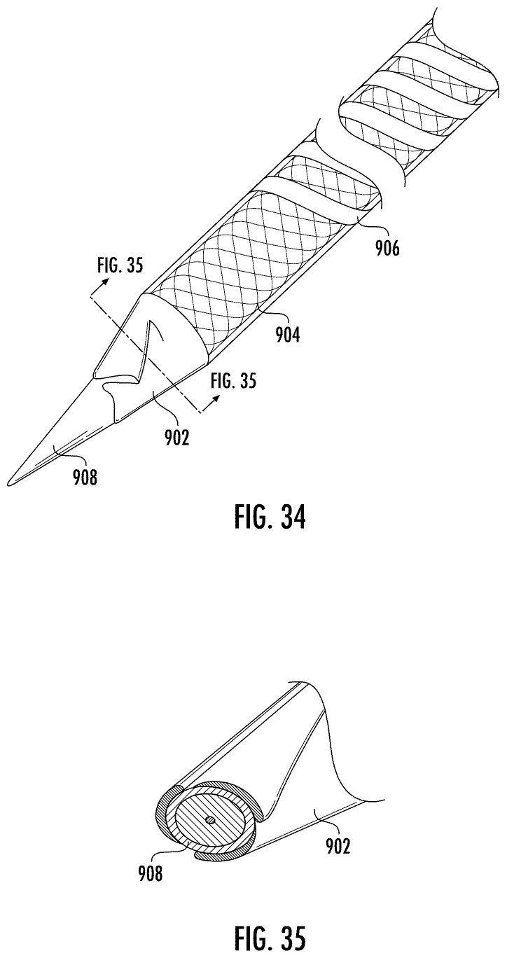

FIGS. 34-35 show a sheath aspect, including a distal end portion 902, which can be an extension of an outer cover extending longitudinally along the sheath in the proximal direction. FIG. 34 shows a distal end portion 902 folded around an introducer (in the crimped and collapsed configuration). FIG. 35 shows a cross section of the distal end portion 902 folded around the introducer 908 (in the crimped and collapsed (or unexpanded) configuration). The distal end portion 902 can be formed of, for example, and without limitation, one or more layers of a similar or the same material used to form the outer layer (liner) of the sheath. Yet, in some implementations, the distal end portion can comprise all the layers disclosed herein. Yet, in some implementations, the distal portion can comprise all the layers disclosed herein with an exemption of the braided layer.

However, there are also aspects where the distal end portion of the sheath can include additional materials that are used in addition or instead of the material similar to the one used in the outer liner. For example, and without limitation, the distal portion of the sheath can include layers of materials that exceed the number of layers in other parts of the sheath. In some aspects, the distal end portion 902 includes an extension of the outer layer of the sheath, with or without one more additional layer added by separate processing techniques. The distal end portion can include anywhere from 1 to 8 layers of material (including 1, 2, 3, 4, 5, 6, 7, and 8 layers of material). In some aspects, the distal end portion comprises multiple layers of a Dyneema® material. The distal end portion 902 can extend distally beyond a longitudinal portion of the sheath that includes braided layer 904 and elastic layer 906. In fact, in some aspects, the braided layer 904 may extend distally beyond the elastic layer 906, and the distal end portion 902 may extend distally beyond both the braided layer 904 and elastic layer 906, as shown in FIGS. 34-35.

The distal end portion 902 may have a smaller collapsed diameter than the more proximal portions of the sheath, giving it a tapered appearance. This smooths the transition between the introducer/dilator and the sheath, ensuring that the sheath does not get lodged against the tissue during insertion into the patient. The smaller collapsed diameter can be a result of multiple folds (for example, 1, 2, 3, 4, 5, 6, 7, or 8 folds) positioned circumferentially (evenly or unevenly spaced) around the distal end portion. For example, a circumferential segment of the distal end portion can be brought together and then laid against the adjacent outer surface of the distal end portion to create an overlapping fold. In the collapsed configuration, the overlapping portions of the fold extend longitudinally along the distal end portion 902. Exemplary folding methods and configurations are described in U.S. Application Number 14/880,109 and U.S. application Ser. No. 14/880,111, each of which are hereby incorporated by reference in their entirety. Scoring can be used as an alternative or in addition to folding of the distal end portion. Both scoring and folding of the distal end portion 902 allow for the expansion of the distal end portion upon the passage of the delivery system and ease the retraction of the delivery system back into the sheath once the procedure is complete. In some aspects, the distal end portion of the sheath (and/or of the vessel dilator) can decrease from the initial diameter of the sheath (e.g., 8 mm) to 3.3 mm (10F) and may decrease to the diameter of a guidewire, allowing the sheath and/or the vessel dilator 300 to run on a guidewire.

In some aspects, a distal end portion can be added, the sheath and tip can be crimped, and the crimping of the distal end portion and sheath can be maintained by the following method. As mentioned above, the distal end portion 902 can be an extension of the outer layer (liner) of the sheath. It can also be a separate, multilayer tubing that is heat bonded to the remainder of the sheath prior to the tip crimping processing steps. In some aspects, the separate, multilayer tubing is heat bonded to a distal extension of the outer layer of the sheath to form the distal end portion 902. For crimping of the sheath after tip attachment, the sheath is heated on a small mandrel. The distal end portion 902 can be folded around the mandrel to create the folded configuration shown in FIG. 34. The folds are added to the distal end portion 902 prior to the tip crimping process or at an intermediate point during the tip crimping process. In some aspects, the small mandrel can be from about 2 millimeters to about 4 millimeters in diameter (including about 2.2 millimeters, about 2.4 millimeters, about 2.6 millimeters, about 2.8 millimeters, about 3.0 millimeters, about 3.2 millimeters, about 3.4 millimeters, about 3.6 millimeters, about 3.8 millimeters, and about 4.0 millimeters). The heating temperature will be lower than the melting point of the material used. This can cause the material to shrink on its own to a certain extent. For example, and without limitation, in some aspects, such as those where Dyneema® materials are utilized as a part of the sheath's outer layer and/or distal end portion materials, a sheath crimping process begins by heating the sheath on a 3 millimeter mandrel to about 125 degrees Celsius (lower than Dyneema@ melting point of about 140 degrees Celsius). This causes the sheath to crimp itself to about a 6 millimeter outer diameter. At this point, the sheath and distal end region 902 are allowed to cool. A heat shrink tube can then be applied. In some aspects, the heat shrink tube can have a melting point that is about the same as the melting point of the distal end portion material. The sheath with the heat shrink tube extending over the sheath and the distal end portion 902 is heated again (for example, to about 125 degrees Celsius for sheaths including Dyneema® outer layers and distal end portions), causing the sheath to crimp to an even smaller diameter. At the distal end portion 902, a higher temperature can be applied (for example, from about 145 degrees Celsius to about 155 degrees Celsius for Dyneema® material), causing the layers of material to melt together in the folded configuration shown in FIG. 34 (the folds can be added at any point during this process). The bonds at the distal end portion 902 induced by the high temperature melting step will still be weak enough to be broken by a passing delivery system. As a final step, the heat shrink tube is removed, and the shape of the sheath remains at the crimped diameter.

FIG. 43 shows a transverse cross section taken near the distal end (a tip) of the sheath in one aspect, at a point longitudinally distal to the braided layer. In such an aspect, the distal end of the sheath does not comprise a braided layer. The distal end of the sheath 501 includes an inner polymeric layer (inner liner) 513, an outer polymeric layer (outer liner) 517, and an outer covering 561. It is understood, however, that an additional layer can be placed between the inner liner and the outer liner. Some of these layers are described in detail below. A method of compressing the distal portion (tip) of an expandable sheath can include: covering at pre-crimped state the distal portion of the expandable sheath 501 with an external covering layer 561 having a melting temperature TM1 which is lower than the melting temperature TM2 of the inner and outer polymeric layers; heating at least one region, which does not span the entire area of overlap between the cover layer 561 and the expandable sheath 501, to a first temperature which is equal or higher than TM2, thereby melting both the covering layer 561 and the outer polymeric layer 517 of the expandable sheath 501, so as to create at attachment region 569 there between; inserting a mandrel into the lumen of the expandable sheath 501 and crimping at least a portion thereof, such as the distal portion, of the expandable sheath 501; heating the external covering layer 561 over the distal portion of the expandable sheath 501 to a second temperature which is at least equal to or higher than the melting temperature TM1 of the external covering layer 561, and lower than the melting temperature TM2 of the inner and outer polymeric layers, for a predefined first time window.

This method advantageously avoids risks that a tear initiated at a score or split line (such as perforation 813 shown in FIG. 29) should divert from the intended axial direction of tear propagation due to defects (weakened points or unintended apertures) in the heat-shrink tubing. This method further enables choosing an external covering layer made of materials that may be heated to form moderately attached folds at temperatures lower than those required for the internal or external layers of the expandable sheath.

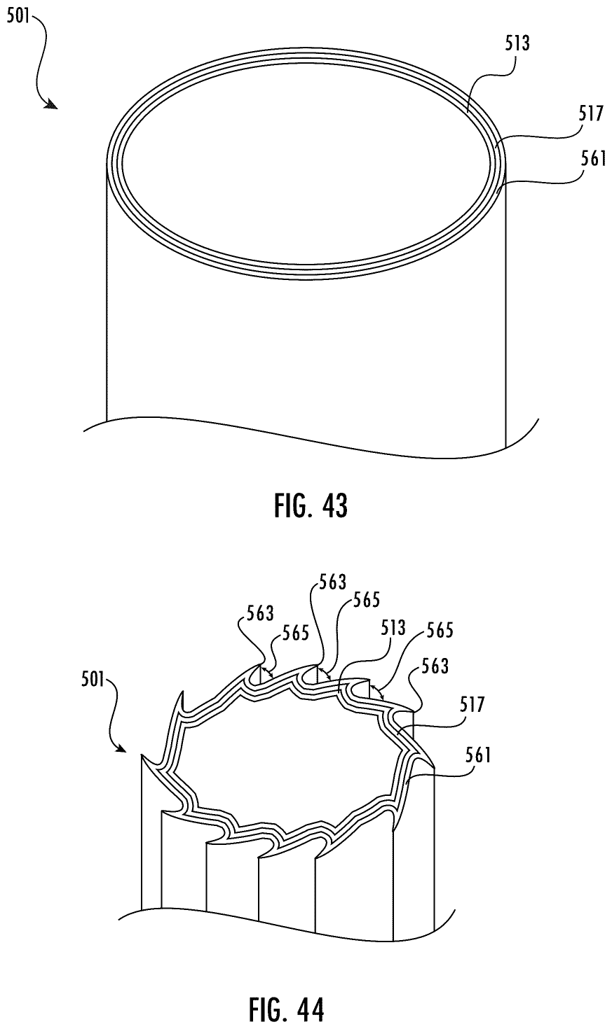

The crimping of the inner and outer polymeric layers (liners) 513, 517 and the external covering layer 561 can be, for example, from a pre-compressed diameter of about 8.3 mm to a compressed diameter of about 3 mm. FIG. 44 shows a transverse cross section of the aspect of FIG. 43 (distal portion (tip) of the sheath) during crimping. Folds 563 are created along the external layer 561 during crimping. The heating to the second temperature is sufficient to melt the external covering layer 561 so as to attach the fold 563 to each other while avoiding similar melting and attachment of the inner and outer polymeric layers. Again, it is understood that additional layers, as described below, can be present between the inner liner and the outer liner of the sheath. In some implementations, it is also understood that the inner liner and/or outer liner, as described in this disclosure, can comprise one or more polymer layers.

The method of compressing the distal portion of the expandable sheath can include a step of covering the expandable sheath 501 and the external covering layer 561 with a heat-shrink tube (HST) prior to, during, or following the heating to the second temperature, wherein the second temperature acts to shrink the HST in order to retain the external covering layer 561 and the expandable sheath 501 in a compressed state. The HST can be removed from the expandable sheath 501, and the external covering layer 561 after the folds 563 of the covering layer 563 are sufficiently attached to each other in the desired compressed state and cooled down for a sufficient period of time.

According to some aspects, the HST is utilized as a heat shrink tape to apply the external radial pressure by wrapping and heating it over the external covering layer 561 and the expandable sheath 501.

According to some aspects, a non-heat-shrink tape can be used instead of a heat-shrink tube.

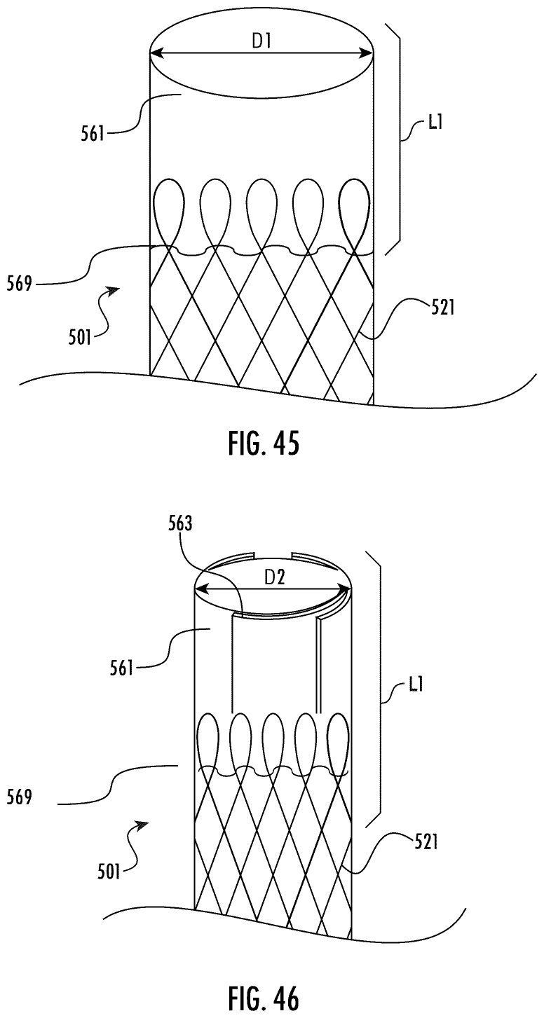

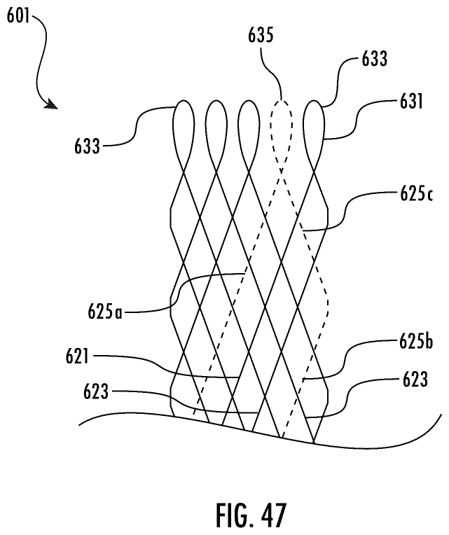

FIG. 45 shows a distal portion of an expandable sheath 501 having an expandable braid 521, wherein its distal portion is covered by an external covering layer 561, which is shown to extend along a length L1 up to the distal edge 513 of the expandable sheath 501. D1 denotes the distal diameter of the expandable sheath 501 in the pre-compressed state. FIG. 46 shows the distal portion of the expandable sheath 501 in a compressed state, wherein its distal diameter D2 is smaller than D1. It should be noted that compressing the external covering layer 561 from an uncompressed state to a compressed state of the expandable sheath 501 results in the formation of folds 563 (FIGS. 44 and 46) along the external covering layer 561 as well as layers 517 and 513 (FIG. 44), when reaching the compressed state, due to the diameter reduction thereof. It is desirable to promote moderate attachment between the folds 563. The term “moderate attachment,” as used herein, refers to an attachment force sufficient in magnitude to form a structural cover maintaining the expandable sheath 501 in a compressed state prior to advancement of a DS component through its lumen, yet low enough so that advancement of the DS component there-through is sufficient to break or disconnect the attachments 565 between the folds 563 (FIG. 44), thereby enabling expansion of the expandable sheath 501.

The external covering layer 561 is chosen such that its melting temperature TM1 is lower than the melting temperature TM2 of the polymeric layers of the expandable sheath 100 in order to promote folds' 563 formation with moderate attachment in the external covering layer 561 while avoiding melting and attaching similar folds in the polymeric layers 513 and 517 of the expandable sheath 501.

According to some aspects, the external covering layer 561 is low-density polyethylene. Other suitable materials, as known in the arts, such as polypropylene, thermoplastic polyurethane, and the like, may be utilized to form the external covering layer 561.

FIGS. 45 and 46 show perspective views of a sheath aspect that is similar to or the same as FIGS. 43 and 44. The external covering layer 561 and expandable sheath 501 were heated to a first temperature TM2 along a circumferential interface therebetween at the proximal end of the external covering layer 561 to form a circumferential proximal attachment region 569.

According to some aspects, the external covering layer 561 is attached to different attachment regions, such as along a longitudinally oriented attachment line, to the external surface of the expandable sheath 501 (e.g., the outer polymeric layer). According to some aspects, the external covering layer 561 is attached to the external surface of the expandable sheath 501 by a plurality of circumferentially spaced attachment regions 569, wherein the circumferential distance between adjacent attachment regions is chosen to allow formation of folds (or pleats) 563 therebetween. Attachment regions 569 ensure that the external covering layer 561 always remains attached to the expandable sheath 501, either during the compressed or expanded states thereof.

According to some aspects, the covering with an external covering layer 561 is performed after crimping the expandable sheath 501, such that the external layer 561 covers pre-formed folds of inner 513 and/or outer 517 layers of the sheath 501.

According to some aspects, the bond between the folds 563 is based on the adhesive with moderate adhesion strength.

Aspects of the sheaths described herein may comprise a variety of lubricious outer coatings, including hydrophilic or hydrophobic coatings and/or surface blooming additives or coatings.

FIG. 27 illustrates aspects of a sheath 500 comprising a tubular inner layer 502. The inner layer 502 may be formed from an elastic thermoplastic material such as nylon and can comprise a plurality of cuts or scorelines 504 along its length such that the tubular layer 502 is divided into a plurality of long, thin ribs or portions 506. When delivery apparatus 10 is advanced through the tubular layer 502, the scorelines 504 can resiliently expand or open, causing the ribs 506 to splay apart and allowing the diameter of layer 502 to increase to accommodate the delivery apparatus.

In some aspects, the scorelines 504 can be configured as openings or cutouts, having various geometrical shapes, such as rhombuses, hexagons, etc., or combinations thereof. In the case of hexagonal openings, the openings can be irregular hexagons with relatively long axial dimensions to reduce foreshortening of the sheath when expanded.

The sheath 500 can comprise an outer layer (not shown), which can comprise a relatively low durometer, elastic thermoplastic material (e.g., Pebax, polyurethane, etc.), and which can be bonded (e.g., by adhesive or welding, such as by heat or ultrasonic welding, etc.) to the inner nylon layer. Attaching the outer layer to the inner layer 502 can reduce the axial movement of the outer layer relative to the inner layer during radial expansion and collapse of the sheath. The outer layer may also form the distal tip of the sheath.

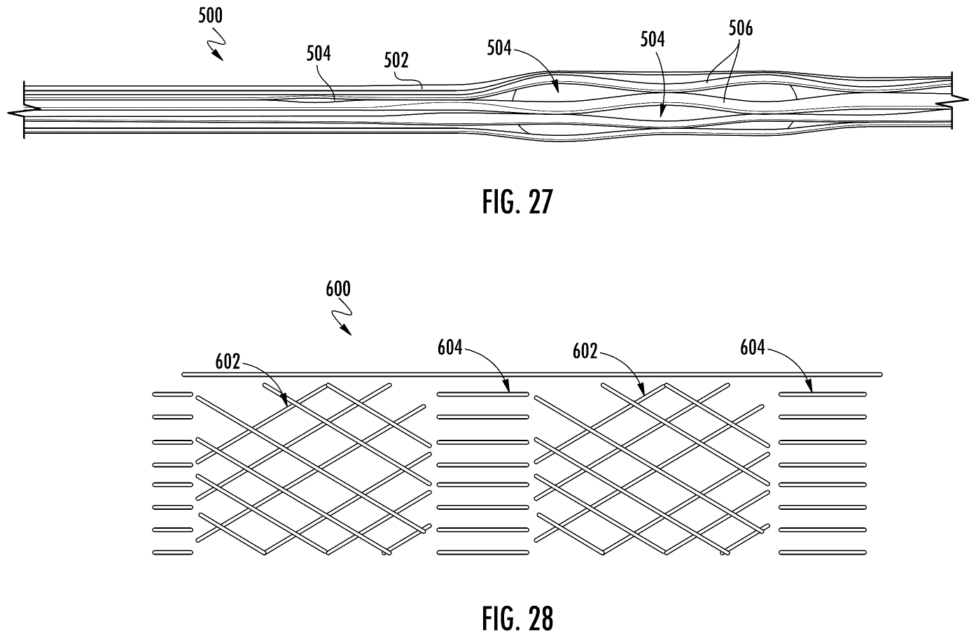

FIG. 28 illustrates some aspects of a braided layer 600 that can be used in combination with any of the sheath aspects described herein. The braided layer 600 can comprise a plurality of braided portions 602, in which filaments of the braided layer are braided together, and unbraided portions 604, in which the filaments are not braided extend axially without being intertwined. In certain aspects, the braided portions 602 and unbraided portions 604 can alternate along the length of the braided layer 600 or may be incorporated in any other suitable pattern. The proportion of the length of the braided layer 600 given to braided portions 602 and unbraided portions 604 can allow the selection and control of the expansion and foreshortening properties of the braided layer.

FIG. 47 depicts an aspect of a braided layer having at least one radiopaque strut or filament. The expandable sheath 601 and its expandable braided layer 621 are shown without the polymeric layers, as would be visualized in the x-ray fluoroscopy for illustration purposes. As shown in FIG. 47, the expandable braided layer 621 comprises a plurality of crossing struts 623, which can form distal crowns 633, for example, in the form of distal loops or eyelets at the distal portion of the expandable sheath 601.

The expandable sheath 601 is configured for advancement in a pre-compressed state up to a target area, for example, along the abdominal aorta or the aortic bifurcation, at which point the clinician should cease further advancement thereof and introduce the DS through its lumen, to facilitate expansion thereof. To that end, the clinician should receive a real-time indication of the expandable sheath's position during the advancement thereof. According to an aspect of the disclosure, there is provided at least one radio-opaque marker at or along at least one region of the expandable braided layer 621, configured to enable visualization of the expandable sheath's position under radio fluoroscopy.

According to one aspect, at least one of the distal crowns 633 comprises a radio-opaque marker. According to some aspects, the distal crowns 633 comprise at least one gold-plated crown 635 (FIG. 47), configured to serve as a radio-opaque marker. It will be clear that gold-plating is merely an example and that the crowns 635 can comprise other radio-opaque materials known in the art, such as tantalum, platinum, iridium, and the like.

Since the expandable sheath 601 comprises an expandable braided layer 621 having a plurality of crossing struts 623 disposed along its length, this structure can be advantageously utilized for more convenient incorporation of radio-opaque elements.

According to some aspects, the struts 623 further comprise at least one radio-opaque strut 625, having a radio-opaque core. For example, a drawn-filled tubing (DFT) wire comprising a gold core (as may be provided by, for example, Fort Wayne Metals Research Products Corp.) may serve as a radio-opaque strut 625. FIG. 47 shows an exemplary expandable braided layer 621 comprising a plurality of less-opaque struts or filaments 623 and radio-opaque struts or filaments 625a, 625b, and 625c. In some instances, the struts 625a and 625c can be made of a single wire, wherein the wire extends along the path of strut 625a, loops at the distal crown 635, and extends along the path of strut 625c therefrom. Thus, a single wire, such as a DFT wire, can be utilized to form radio-opaque struts 625a and 625c and radio-opaque distal crown 635.

Since radio-opaque wires, such as a DFT wire, can be costly, the expandable braided layer 621 can comprise a plurality of non-radio-opaque or less radio-opaque struts 623, for example, made of a shape-memory alloy such as Nitinol and polymer wire such as PET respectively, intertwined with at least one radio-opaque strut 625 (FIG. 47).

According to some aspects, radio-opaque wires are embedded within the polymer braid, such as the outer polymeric layer 617 or the inner polymeric layer 615, which are made of less-opaque materials.

Advantageously, the expandable braid embedded within the expandable sheath is utilized according to the disclosure for incorporating radio-opaque markers along specific portions thereof to improve visualization of the sheath's position in real-time under radio fluoroscopy.

According to yet some aspects of the disclosure, radiopaque tubes can be threaded on the distal crowns or loops 633, or radiopaque rivets can be swaged on the distal crowns or loops 633 to improve their visibility under fluoroscopy.

FIG. 36 shows a longitudinal cross section of some aspects of expandable sheath 11 (positioned on mandrel 91 during the fabrication process, under compression by heat shrink tube 51). The sheath 11 comprises a braided layer 21 but lacks the elastic layer described in the previous aspects. The heat applied during the shrinking procedure may promote at least partial melting of the inner 31 and outer 41 polymeric layers (liners). In some aspects, if additional layers are present between the inner and outer liners, the shrinking procedure can also promote at least a partial melting of those layers. Since the filaments of the braid define open cells therebetween, uneven outer surfaces may be formed when the inner 31 and outer 41 polymeric layers melt into the cell openings and over the filaments of the braided layer 21.

In order to mitigate uneven surface formations, cushioning polymeric layers 61a, 61b are added between the inner 31 and outer 41 layers of the sheath 11, configured to evenly spread the forces acting in the radial direction during sheath compression. A first cushioning layer 61a is placed between the inner polymeric layer 31 and the braided layer 21, and a second cushioning layer 61b is placed between the outer polymeric layer 41 and the braided layer 21. In some aspects, the cushioning polymeric layers 61a and 61b are sacrificial and are removed in a later processing step.

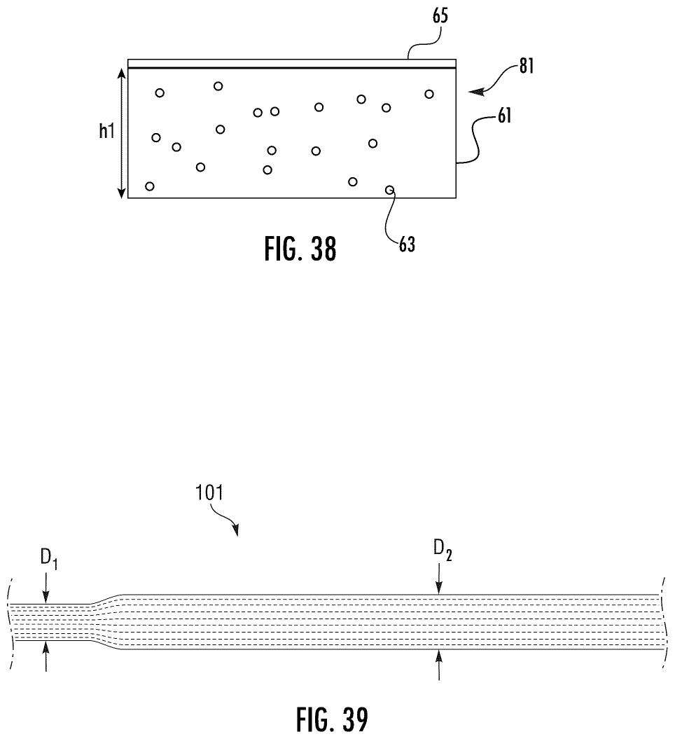

The cushioning layers 61a, 61b can comprise a porous material having a plurality of micropores of nanopores 63 (FIGS. 37-38) in a porous interior region. One such material includes, but is not limited to, expanded polytetrafluoroethylene (ePTFE). A porous cushioning layer can advantageously be formed with a minimal thickness h1 required to sufficiently spread the compression forces to prevent uneven surface formation along the inner 31 and outer 41 polymeric layers. Thickness h1 is measured in the radial direction (from an inner surface to an outer surface) of the cushioning layer and can be from about 80 microns to about 1,000 microns (including, for example, about 80 microns, about 90 microns, about 100 microns, about 110 microns, about 120 microns, about 130 microns, about 140 microns, about 150 microns, about 160 microns, about 170 microns, about 180 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, and about 1,000 microns). In some aspects, the range of thickness h1 is from about 110 to 150 microns.

However, when cushioning layers comprise a plurality of micropores of nanopores 63 (FIGS. 37-38), the inner 31 and outer 41 polymeric layers may melt into the pores of the cushioning layers 61a, 61b upon heating during the fabrication process. In order to prevent the inner 31 and outer 41 polymeric layers from melting into the pores 63 of the cushioning layer 61, a first sealing layer 71a can be placed between the inner polymeric layer 31 and the first cushioning layer 61a, and a second sealing layer 71b can be placed between the outer polymeric layer 41 and the second cushioning layer 61b (as shown in FIG. 36). The sealing layers 71a, 71b can have a higher melting point than the polymeric layers 31 and 41 and can be formed of a non-porous material (such as, but not limited to, polytetrafluoroethylene) in order to prevent fluid flow therethrough. The thickness h2 of each sealing layer 71 (FIG. 37), measured in a radial direction from the inner to the outer surface of the sealing layer, can be much thinner than that of the cushioning layer 61, for example, from about 15 to about 35 microns (including about 15 microns, about 20 microns, about 25 microns, about 30 microns, and about 35 microns). In some aspects, the sealing layers 71a and 71b are sacrificial and are removed in a later processing step.

While advantageous for the reasons described above, the addition of the cushioning and sealing can increase the complexity and time required to assemble the sheath 11. Advantageously, providing a single sealed cushioning member configured to provide both cushioning and sealing functionalities (instead of providing two separate cushioning and sealing layers, each configured to provide one functionality) reduces sheath assembly time and significantly simplifies the process. According to an aspect of the disclosure, there is provided a single sealed cushioning member configured for placement between the inner and outer polymeric layers of the sheath and the central braided layer. The single sealed cushioning member includes a cushioning layer and a sealed surface configured to prevent leakage/melting into the pores in the radial direction.

FIG. 37 shows an aspect of a single sealed cushioning member 81′, comprising a cushioning layer 61 having a width thickness h1 as elaborated hereinabove, fixedly attached to a corresponding sealing layer 71 having a thinner thickness h2 to form the sealed surface. The sealing layer 71 and the cushioning layer 61 are pre-assembled or pre-attached to each other to form together a single member 81′, for example, by gluing, welding, and the like.

FIG. 38 shows one aspect of a single sealed cushioning member 81, comprising a cushioning layer 61 having a width thickness h1, wherein the cushioning layer 61 is provided with at least one sealed surface 65, configured to face an inner 31 or an outer 41 polymeric layer when assembled in the sheath 11. According to some aspects, the sealed surface 65 can be formed by a surface treatment configured to fluidly seal a surface of the cushioning layer 61. As such, the sealed surface 65 can be the same material as the cushioning layer 61.

According to some aspects of the disclosure, and as mentioned above, with respect to FIG. 36, a minimum of three layers may be sufficient to retain the sheath's expandability, provided with the preferable resistance to axial elongation. This is accomplished by eliminating the need to incorporate an additional elastic layer in the sheath, thereby advantageously reducing production costs and simplifying manufacturing procedures.

The sheath does not necessarily return to an initial diameter but may rather remain in an expanded diameter upon passage of the valve in the absence of the elastic layer.

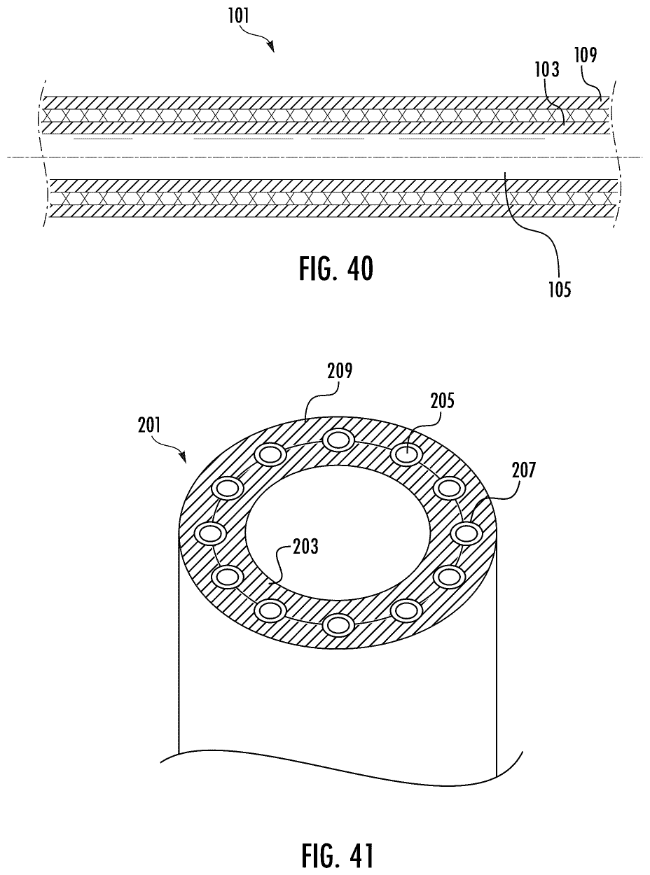

FIGS. 39-40 show an expandable sheath 101 similar to the expandable sheath 100 shown in FIG. 3, but without an elastic layer 106. The inner and outer layers 103 and 109 may be structured and configured to resist axial elongation of the sheath 101 during expansion. However, in the proposed configuration, the absence of an elastic layer results in the sheath 101 remaining in an expanded diameter along the sheath's portion proximal to the valve, without necessarily collapsing back to the initial diameter D₁ after the valve passes in in the longitudinal direction. FIG. 39 is a schematic representation of the sheath 101 remaining in an expanded diameter D₂ along the portion proximal to the valve's passage.

In some aspects, provided herein is an expandable sheath for deploying a medical device, comprising a first polymeric layer, a braided layer radially outward of the first polymeric layer, and a second polymeric layer radially outward of the braided layer. The braided layer includes a plurality of filaments braided together. The second polymeric layer can be bonded to the first polymeric layer such that the braided layer is encapsulated between the first and second polymeric layers. When a medical device is passed through the sheath, the diameter of the sheath expands from a first diameter to a second diameter around the medical device, while the first and second polymeric layers resist axial elongation of the sheath such that the length of the sheath remains substantially constant. However, according to some aspects, the first and second polymeric layers are not necessarily configured to resist axial elongation.

According to some aspects of the disclosure, the expandable sheath does include an elastic layer. But, unlike elastic layer 106, shown in FIG. 3, the elastic layer is not configured to apply a substantial radial force. It can still serve to provide column strength to the sheath. By limiting the tangential (diametrical) expansion of the braid, the elastic layer enhances the strength of the braid and the sheath in the axial direction (column strength). As such, using elastic materials with higher tensile strengths (resistance to stretch) will result in a sheath with greater column strength. Likewise, elastic materials that are under greater tension in the free state will also result in a sheath with greater column strength during pushing, as they will be more resistant to stretch. The pitch of any helically wound elastic layers is another variable that contributes to the column strength of the sheath. The additional column strength ensures that the sheath does not spontaneously expand due to frictional forces applied thereto during forward movement in a distal direction and does not buckle when the delivery system is pulled out of the sheath.

In some optional aspects, the elastic layer can be applied by dip coating in the elastic material (such as, but not limited to) silicone or TPU. The dip coating can be applied to the polymeric outer layer or to the braided layer.

Thus, there is provided an expandable sheath for deploying a medical device, comprising a first polymeric layer, a braided layer radially outward of the first polymeric layer, an elastic layer radially outward of the braided layer, and a second polymeric layer radially outward of the braided layer. The braided layers comprise a plurality of filaments braided together. The elastic layer is configured to provide the expandable sheath with sufficient column strength to resist buckling of spontaneous expansion due to friction forces applied thereto by a surrounding anatomical structure during the sheath's movement in an axial direction. The second polymeric layer is bonded to the first polymeric layer such that the braided layer is encapsulated between the first and second polymeric layers. When a medical device is passed through the sheath, the diameter of the sheath expands from a first diameter to a second diameter around the medical device, optionally, while the first and second polymeric layers resist axial elongation of the sheath such that the length of the sheath remains substantially constant.

According to an aspect of the disclosure, there is provided a three-layered expandable sheath comprising an inner polymeric layer, an outer polymeric layer bonded to the inner polymeric layer, and a braided layer encapsulated between the inner and outer polymeric layers, wherein the braided layer comprises an elastic coating.

FIG. 41 shows a transverse cross section of expandable sheath 201. The expandable sheath 201 includes inner and outer polymeric layers 203 and 209 and a braided layer 205. Instead of the elastic layer described with reference to FIG. 3 above, the braided layer 205 is provided with an elastic coating 207. The elastic coating 207 can be applied directly to the filaments of the braided layer 205, as shown in FIG. 41. The elastic coating can be made of synthetic elastomers, exhibiting properties similar to those described in conjunction with the elastic layer 106.

In some aspects, the second outer polymeric layer 209 is bonded to the first, inner polymeric layer 203, such that the braided layer 205 and the elastic coating 207 are encapsulated between the first and second polymeric layers. Moreover, the elastic coating applied directly to the braided filaments is configured to serve the same function as that of the elastic layer 106 (that is, to apply radial force on the braided layer and the first polymeric layer).

While the aspect of FIG. 41 shows the elastic coating 207 covering the entire circumference of every filament of the braided layer 205, it will be understood that only a portion of the filaments, for example, a portion constituting essentially an outer surface of the braided layer, may be coated by the elastic coating 207.

Alternatively, or additionally, an elastic coating can be applied to other layers of the sheath.

In some aspects, a braided layer 105, such as the one shown in FIG. 39 and FIG. 40 can have a self-contractible frame made of a shape-memory material or a material that exhibits superelastic properties under specific conditions, such as, but not limited to, Nitinol. The self-contracting frame can be pre-set to have a free-state diameter equal to the sheath's initial compressed diameter D1, for example, prior to being placed on a mandrel around the first polymeric layer. The self-contracting frame may expand to a larger diameter D2 while an inner device, such as a prosthetic valve, passes through the sheath's lumen and self-contract back to the initial diameter D1 upon passage of the valve. In some aspects, the filaments of the braid are the self-contracting frame and are made of a shape-memory material.

According to some aspects, an expandable sheath can include a braided expandable layer attached to at least one expandable sealing layer. In some aspects, the braided layer and the sealing layer are the only two layers of the expandable sheath. The braided layer is passively or actively expandable relative to a first diameter, and the at least one expandable sealing layer is passively or actively expandable relative to a first diameter. An expandable sealing layer can be useful with any of the aspects described above and may be particularly advantageous for braids having self-contracting frames or filaments.

The braided layer can be attached or bonded to the expandable sealing layer along its entire length, advantageously decreasing the risk of the polymeric layer being peeled off the braided layer due to frictional forces that may be applied thereon either during entry or exit through the surgical incision. The at least one sealing layer can comprise a lubricious, low-friction material so as to facilitate passage of the sheath within the blood vessels and or to facilitate passage of the delivery apparatus carrying a valve through the sheath.

A sealing layer is defined as a layer that is not permeable to the blood flow. The sealing layer can comprise a polymeric layer, a membrane, a coating, and/or a fabric, such as a polymeric fabric. According to some aspects, the sealing layer comprises a lubricious, low-friction material. According to some aspects, the sealing layer is radially outward to the braided layer so as to facilitate passage of the sheath within the blood vessels. According to some aspects, the sealing layer is radially inward to the braided layer so as to facilitate passage of the medical device through the sheath.

According to some aspects, the at least one sealing layer is passively expandable and/or contractible. In some aspects, the sealing layer is thicker at certain longitudinal positions of the sheath than at others, which can hold a self-contracting braided layer open at a wider diameter than at other longitudinal positions where the sealing layer is thinner.

Attaching the braided layer to at least one expandable sealing layer instead of encapsulating it between two polymeric layers bonded to each other may simplify the manufacturing process and reduce costs.

According to some aspects, the braided layer can be attached to both an outer expandable sealing layer and an inner expandable sealing layer so as to seal the braided layer from both sides while facilitating passage of the sheath along the blood vessels and facilitating passage of a medical device within the sheath. In such aspects, the braided layer can be attached to a first sealing layer, while the other sealing layer may also be attached to the first sealing layer. For example, the braided layer and the inner sealing layer can be each attached to the outer sealing layer, or the braided layer and the outer sealing layer can be each attached to the inner sealing layer.

According to some aspects, the braided layer is further coated by a sealing coating. This may be advantageous in configurations of a braided layer being attached only to a single expandable layer, wherein the coating ensures that the braided layer remains sealed from the blood flow or other surrounding tissues, even along regions that are not covered by the expandable layer. For example, if a braided layer is attached to a sealing layer on one side, the other side of the braided layer may receive a sealing coating. In some aspects, the sealing coating can be used instead of, or in addition to, one or both of the sealing layers.

According to some aspects, an expandable sheath for deploying a medical device are also disclosed. It is understood that in these exemplary aspects, the sheath can comprise any of the layers, elements, or materials described above. It is also understood that any of the described above methods of making the sheath can also be applied to disclosed below exemplary sheaths. Similarly, the methods of making the pleats (folds), such as crimping procedures, are also applicable to the disclosed below exemplary sheaths.

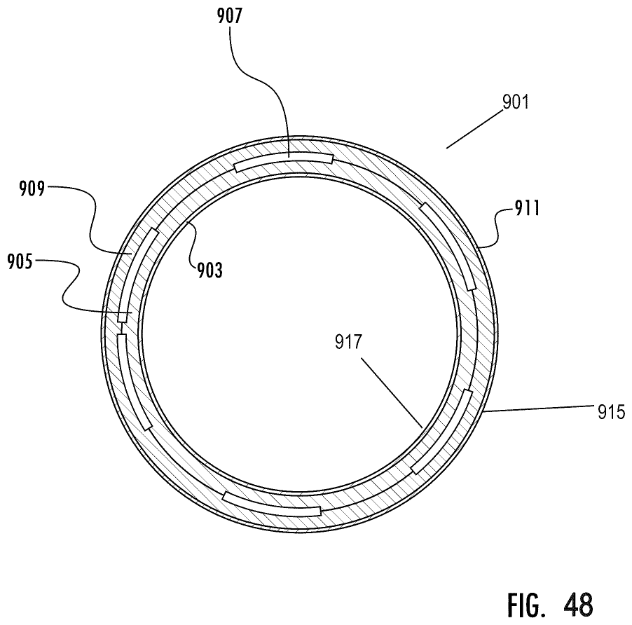

Some exemplary aspects are shown in FIGS. 48-52. An exemplary sheath disclosed herein has a proximal end and a distal end, an inner surface, and an outer surface. FIG. 48 shows a cross-section of such an exemplary sheath 901. The exemplary sheath 901 (FIG. 48) has an inner surface 917 and an outer surface 915.

In some aspects, an expandable sheath 901 shown in FIG. 48 further comprises an inner low-friction liner 903 having a first surface and an opposite second surface, where the first surface of the inner liner defines the inner surface 917 of the sheath 901. It is understood that the inner liner can comprise one or more polymer layers. In some aspects, the inner liner can comprise two or more layers. In some implementations, the inner liner can comprise from 1 to 8 layers, including an exemplary amount of 2, 3, 4, 5, 6, and 7 layers. It is understood that the inner liner can also comprise more than 8 layers, for example, and without limitation, it can comprise 9, 10, 15, 20, or more than 25 layers. It is understood that in some aspects, the layers of the polymers can be melted together during manufacturing processes.

In some implementations, if desired, an additional low friction polymer layer, such as PTFE, for example (not shown), can be disposed on the first surface of the inner liner. In such an exemplary aspect, the PTFE layer would define the inner surface of the sheath.

In some implementations, the exemplary sheath 901 further comprises an outer low-friction liner 911 having a first surface and an opposite second surface, wherein the second surface of the outer liner defines the outer surface 915 of the sheath 901. It is also understood that similar to the inner liner 903, the outer liner 911 can comprise one or more polymer layers. Yet, in some implementations, the outer liner 911 can comprise two or more polymer layers. In some implementations, the outer liner can comprise from 1 to 8 layers, including an exemplary amount of 2, 3, 4, 5, 6, and 7 layers. It is understood that the outer liner can also comprise more than 8 layers, for example, and without limitation, it can comprise 9, 10, 15, 20, or more than 25 layers. It is understood that in some aspects, the layers of the polymers can be melted together during manufacturing processes.

In some implementations, the outer liner 911 can further comprise any of the disclosed herein hydrophilic coatings.

The sheath 901 can further comprise a first polymeric layer 905 that surrounds radially outward of the inner liner 903, such that it overlies the second surface of the inner liner 903. In the aspects disclosed herein, the first polymeric layer can comprise one or more sublayers. Yet, in some implementations, the first polymeric layer can comprise two or more polymeric sublayers. For example, the first polymeric layer can comprise from 1 to 8 sublayers, including an exemplary amount of 2, 3, 4, 5, 6, and 7 sublayers. It is understood that the first polymeric layer can also comprise more than 8 sublayers, for example, and without limitation, it can comprise 9, 10, 15, 20, or more than 25 sublayers. It is understood that in some aspects, the sublayers of the first polymeric layer can be melted together during manufacturing processes.

In some implementations, the sheath 901 can further comprise a braided layer 907 that is disposed radially outward of the first polymer layer 905.

In some implementations, the sheath 901 can further comprise a second polymeric layer 909 that surrounds radially outward of the braided layer 907. In the aspects disclosed herein, the second polymeric layer can comprise at least one sublayer or two or more polymeric sublayers. For example, the second polymeric layer can comprise from 1 to 8 sublayers, including an exemplary amount of 2, 3, 4, 5, 6, and 7 sublayers. It is understood that the second polymeric layer can also comprise more than 8 sublayers, for example, and without limitation, it can comprise 9, 10, 15, 20, or more than 25 sublayers. It is understood that in some aspects, the sublayers of the second polymeric layer can be melted together during manufacturing processes. As disclosed herein, the first surface of the outer liner 911 overlies the second polymeric layer 909.

FIG. 48 is shown as an exploded view for exemplary purposes. In practice, adjacent layers will be in contact with each other and, in some cases, laminated such that they meld with or extend through each other. In some implementations, the layers of this exemplary sheath form a laminate structure.

For example, and without limitations, any of the disclosed herein braided layers can be used as the braided layer 907. In certain exemplary aspects, the braided layer can comprise a plurality of helical multifilar filaments braided together. In such aspects, the first and second polymeric layers 905, 909 can be thermally bonded to each other through the open spaces of the braided layer 907 such that the braided layer is encapsulated between these two polymeric layers. In some implementations, the first and second polymeric layers 905, 909 can also be thermally bonded to the adjacent inner and outer liners 903, 911. In such aspects, the braided layer is encapsulated between all the layers of the sheath. In some aspects, and as disclosed herein, the inner and outer liners can comprise various polymeric materials. In certain aspects, these polymeric materials can be porous. In such exemplary aspects, the first and second polymeric layers can penetrate at least a portion of the pores present in the porous material of the inner and/or outer liners during the manufacturing process. As a result, the sheath is more mechanically stable than any other known in the art sheaths.

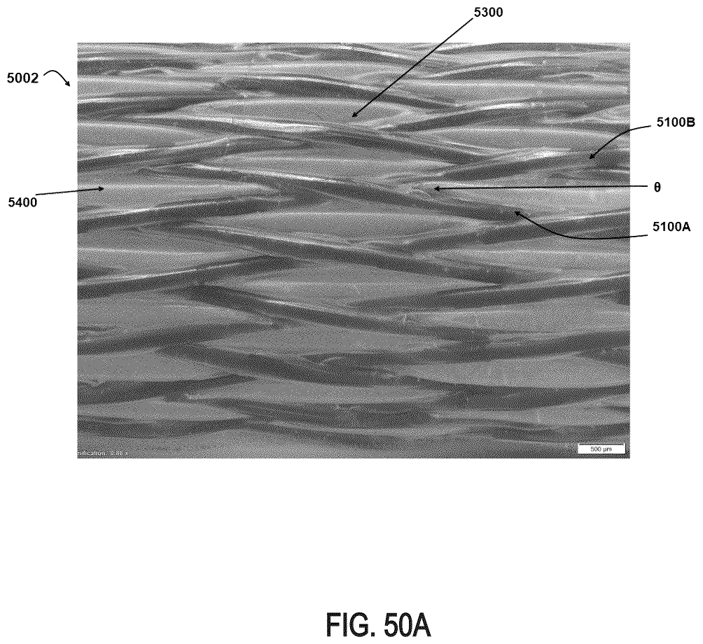

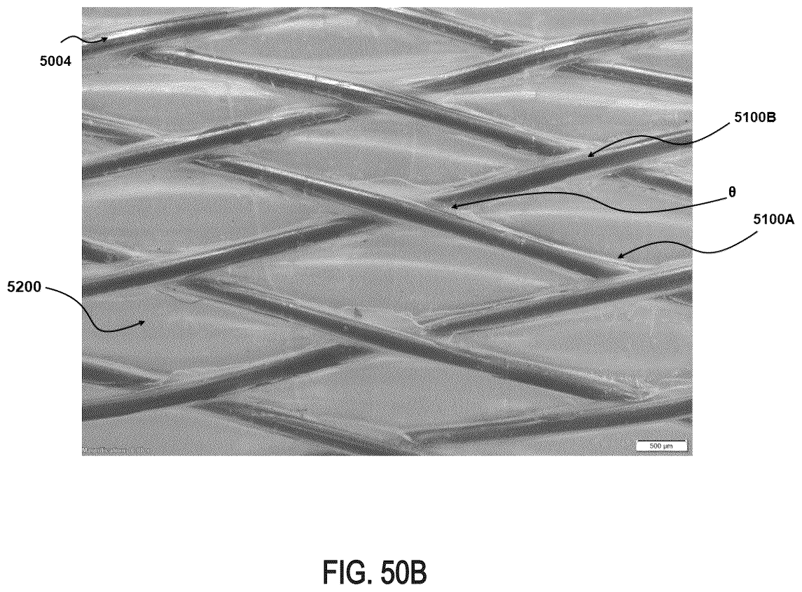

As disclosed in detail above, when the first polymeric layer, second polymeric layer, inner liner, and outer liner encapsulate the braided layer, they can connect (adhere or penetrate if the porous materials are present) to each other through the spaces between the filaments of the braided layer as shown above (for example as shown in FIGS. 50A-50B). In some implementations, the layers of the sheath can also be bonded (adhered or penetrated into the pores if present) together at the proximal and/or distal ends of the sheath.

In some implementations, it is understood that the filaments of the braided layer are not adhered to the polymeric layers of the sheath. This can allow the filaments, similarly to the aspects disclosed above, to move angularly relative to each other and relative to the first and the second polymeric layers, as well as relatively to all polymeric layers of the laminate structure, allowing the diameter of the braided layer, and thereby the diameter of the sheath, to increase or decrease upon the passage of the medical device. Again, as disclosed above, in this exemplary sheath, the angle θ between the filaments can change, and the length of the braided layer can also change. For example, as the angle θ increases, the braided layer can foreshorten, and as the angle θ decreases, the braided layer can lengthen to the extent permitted by the areas where the polymeric layers of the laminate structure are bonded. However, because the braided layer is not adhered to the polymeric layers of the sheath, the change in length of the braided layer that accompanies a change in the angle θ between the filaments does not result in a significant change in the length L of the sheath.

In some implementations, the laminate structure of this exemplary sheath can facilitate resistance to kinking and ballooning.

In some implementations, the inner and outer liners can comprise any of the disclosed above materials. In certain aspects, the inner liner comprises a first material. In some implementations, the outer liner can comprise a fourth material. In certain exemplary aspects, the first and fourth materials can be the same or different.

In some aspects, the material (the first and the fourth) of a low friction inner and outer liners 903, 911 can be a material with a relatively low coefficient of friction yet a relatively high tensile strength. The inner and/or outer liners can have a coefficient of friction of less than about 0.3, less than about 0.2, less than about 0.1, less than about 0.09, less than about 0.08, less than about 0.07, less than about 0.06, less than about 0.05, less than about 0.04, less than about 0.03, less than about 0.02, or even less than about 0.01.

In some exemplary aspects, materials for the inner and outer liners (the first and the fourth) can comprise ultra-high molecular weight polyethylene (UHMWPE). In some implementations, the UHMWPE can be present as a fabric, a laminate, or a porous film or membrane. For example, the inner and outer liners can comprise or be formed of Dyneema® UHMWPE. In some aspects, the inner and outer liner can comprise, or be formed of, the Dyneema Purity® membrane, which has a tensile strength of about 20 MPa. In some exemplary and unlimiting aspects, the inner and outer liner can also be formed by coating. In such aspects, the UHMWPE can be provided as a polymeric solution, for example. Any known in the art coating methods can be utilized. For example, the coating methods can include dipping, doctor blade coating, spraying, and the like.

Other suitable materials for the inner and outer liners (in addition or instead of those disclosed above) can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Again, it is understood that these materials can also be provided in any known in the art form.

In some implementations, the first polymeric layer and the second polymeric layer can also comprise any of the disclosed herein materials. In some aspects, the first polymeric layer and the second polymeric layer can be the same or different. In some aspects, the first polymer layer can comprise a second material, while the second polymeric layer can comprise a third material. Again, as mentioned above, the second material and the third materials can be the same or different. In certain aspects, the second material and/or the third material can comprise polyolefin or polyurethane. In some implementations, the polyolefin can comprise polyethylene, polypropylene, or a combination thereof. In some implementations, where the second material and/or the third material are polyolefin, such a polyolefin can comprise a bi-oriented polypropylene, cast polypropylene, a low-density polyethylene (LDPE), or a high density polyethylene (HDPE), or any combination thereof. In some implementations, where the second material and/or the third material are polyurethane, such aspects comprise a thermoplastic polyurethane.

It is understood that the second and/or the third materials can be provided in any form known in the art. In some aspects, they can be provided as a film or as a solution. In such aspects, if the materials are provided as a solution, the first and the second polymeric materials can be formed by coating, for example, dipping, spraying, doctor blade coating, and the like.

In some implementations, the tensile strength of the first polymeric layer and/or the second polymeric layer is substantially the same or different from the tensile strength of the inner liner and/or outer liner. In some implementations, the tensile strength of the first polymeric layer and/or the second polymeric layer can be larger than the tensile strength of the inner liner and/or outer liner. In some implementations, the tensile strength of the first polymeric layer and/or the second polymeric layer can be smaller than the tensile strength of the inner liner and/or outer liner.

In some exemplary aspects, the porous structure of inner and outer liners, the liner 903, 911 can enable the first and second polymeric layers 905, 909 to flow into the pores during processing to mechanically bond the layers together. Such a laminate structure allows the sheath to be more mechanically stable and durable. In such exemplary aspects, the inner liner and the outer liner can exhibit a mechanical strength higher than a mechanical strength of a reference sheath that does not comprise a substantially identical laminated structure. In some implementations, the disclosed herein sheath can exhibit an improved column strength when compared with a substantially identical reference sheath in the absence of a laminate structure.

In some aspects, liners 903, 911 can be relatively thin compared to the radial thickness of the adjacent first and second polymeric layers, having the appearance of a liner or a membrane. For example, the liner can have a radial thickness ranging from about 0.5 microns to about 40 microns, including about 1 micron, about 2 microns, about 3 microns, about 4 microns, 5 microns, about 10 microns, about 15 microns, about 20 microns, about 25 microns, about 30 microns, about 35 microns, and about 40 microns.

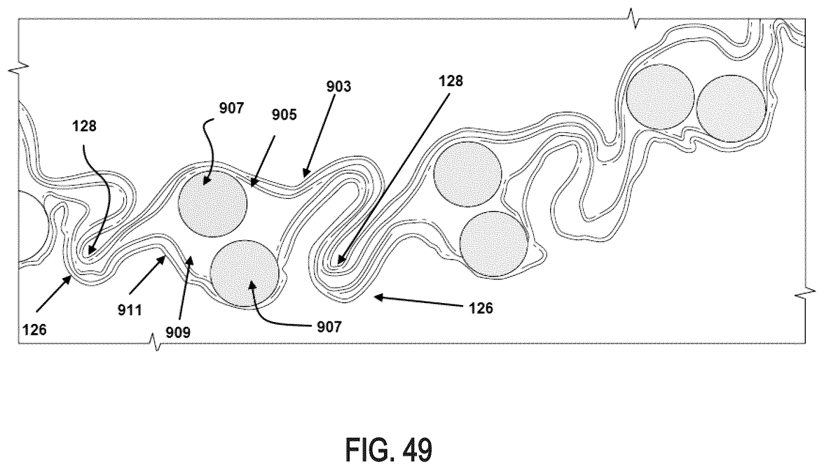

In some implementations and as illustrated in FIG. 49, the sheath can comprise a plurality of pleats of creases that extend along at least a portion of the circumference of the sheath and along its length. Similar pleats (folds, creases having ridges, and valleys 126/128) are shown in FIG. 3, for example. FIG. 49 shows a cross section of a portion of the sheath. It can be seen that the braid filaments 907 are encapsulated between inner and outer liners 903 and 911 and the first and the second polymers 905 and 907, respectively. The pleats having ridges 126 and valleys 128 are spaced circumferentially along the sheath. It is understood that each of the plurality of pleats can comprise at least a portion of the inner liner, and/or at least a portion of the first polymeric layer, and/or at least a portion of the braided layer, and/or at least a portion of the second polymeric layer, and/or at least a portion of the outer liner. These pleats are configured to flatten out when the sheath is in an expended state and form back after collapsing back to the unexpended state. It is understood that these pleats can have a random pattern around the circumference of the sheath or along a length of the sheath. It is also understood that these pleats can have a random longitudinal pattern that is not the same at different portions of the sheath's length. In some implementations, the plurality of pleats can extend along just a central portion, along just a proximal portion, or close to a distal portion, or along of any combination thereof.

The plurality of pleats, as shown in FIG. 49, in some aspects, can extend longitudinally along just a portion of the sheath or along the entire length of the sheath. When the sheath is in a collapsed (unexpanded state, the circumferentially spaced pleats can form a plurality 126 of ridges circumferentially spaced apart from each other by valleys 128. It is understood that these circumferentially spaced pleats can be extended longitudinally in an orderly or random fashion.

Yet, in some implementations, the pleats can be structured circumferentially or longitudinally in any desired pattern. It is also understood that these pleats along the length of the sheath are formed as the various polymer layers encapsulate the braid during the manufacturing proceedings and are flattening out or shortened as a result of braid expansion.

In other words, in some aspects, the plurality of pleats can be uniformly distributed along at least a portion of a length of the sheath and at least a portion of a circumference of the sheath; while in some implementations, the plurality of pleats can be randomly distributed along at least a portion of a length of the sheath and at least a portion of a circumference of the sheath.

In some implementations, as the medical device is passed through the sheath, the ridges and valleys of the pleats can at least partially level out to allow a sheath wall to radially expand and allow the medical device to pass through without damaging the sheath or vascular system of the patient. The photograph of the locally collapsed and locally expanded sheath is shown in FIGS. 50A-50B, respectively.

It can be seen in FIG. 50A that in the collapsed state, the sheath 5002 comprises a braided layer having the filaments 5100A and 5100B (arranged similar to the filaments shown in FIG. 5B) having an angle θ. The plurality of pleats form ridges 5400 and valleys 5300 around the filaments of the braided layer. When the sheath is in the expanded state 5004, as shown in FIG. 50B, the plurality of the pleats straightens out as shown in 5200, while the angle θ between the filaments 5100A and 5100B increases. It is understood that angle θ can have any value between about 5° to about 70°, including exemplary values of about 10°, about 15°, about 20°, about 25°, about 30°, about 35°, about 40°, about 45°, about 50°, about 55°, about 60°, and about 65° depending on whether the sheath in the collapsed, partially collapsed, partially expanded, or expanded state.

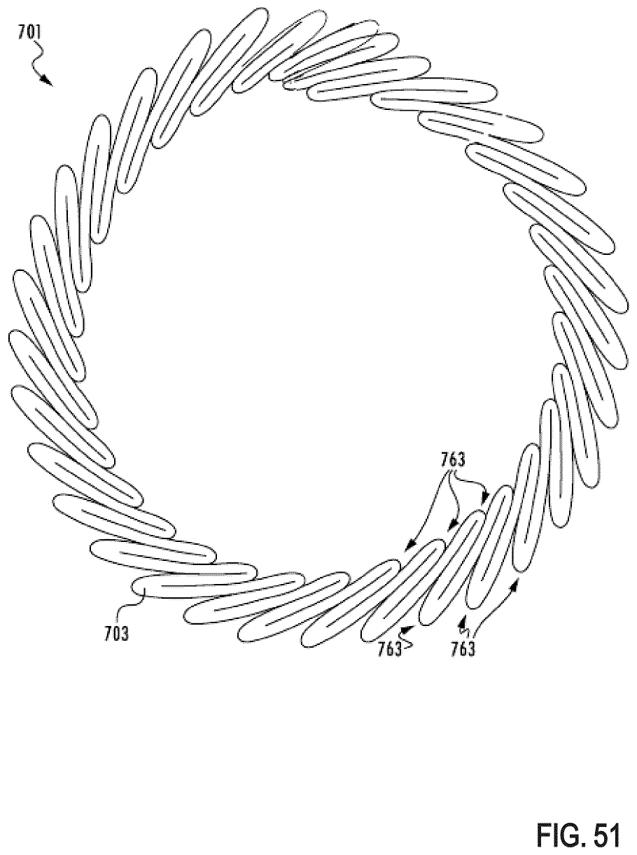

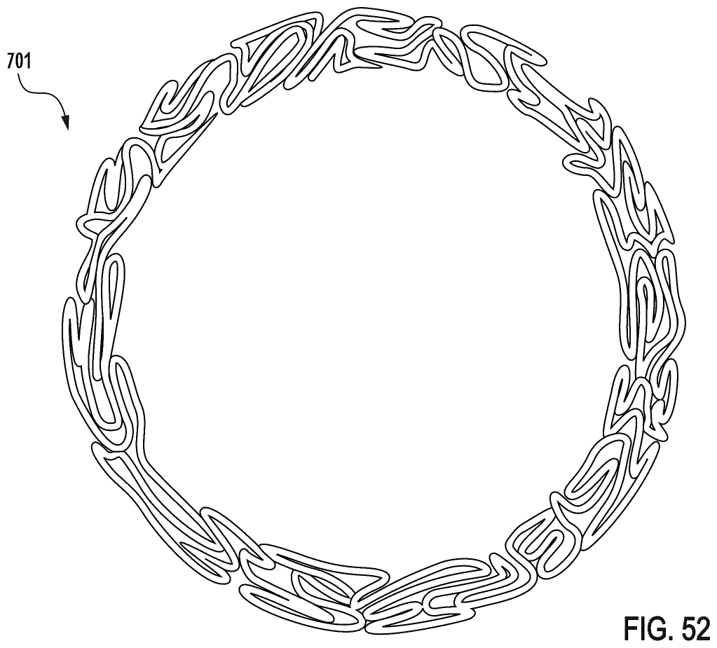

FIGS. 51 and 52 show a distal end or a tip of the sheath. On the distal end of the sheath, the pleats can have a more organized structure of the pleats. It is understood that the sheath's tip may not comprise a braid, and therefore, the pleats are not dependent on the braid structure and the state. As shown, the tip (or distal portion of the sheath) can comprise a plurality of longitudinally extending pleats (fold). As shown in FIG. 51, the collapsed (unexpanded) wall 703 of a sheath 701 can include a plurality of pleats 763. It is understood that each of the plurality of pleats can comprise at least a portion of the inner liner, at least a portion of the first polymeric layer, at least a portion of the second polymeric layer, and at least a portion of the outer liner.

The folds can be arranged in an orderly fashion, as shown in FIG. 51, but are not necessarily so. In some aspects, manufacturing processes lead to more randomly arranged longitudinally-extending folds, valleys, and ridges, as shown in FIG. 52.

As a medical device passes through the inner lumen of the tip, it applies an outward radial force on the sheath wall 703. This causes the plurality of longitudinally-extending pleats 763 to partially or fully unfold (or at least partially smooth out) (that is, a single longitudinally-extending pleat may partially or fully unfold, and several of the longitudinally-extending pleats may partially or fully unfold, or all of the longitudinally-extending pleats may partially or fully unfold).

In some implementations, an additional lubricious liner can be used. In such aspects, this liner can be applied to the first surface of the sheath and becomes the most inner surface of the sheath. In such aspects, this additional lubricious liner can comprise a low coefficient of friction materials that can facilitate the passage of the medical device through the inner lumen.

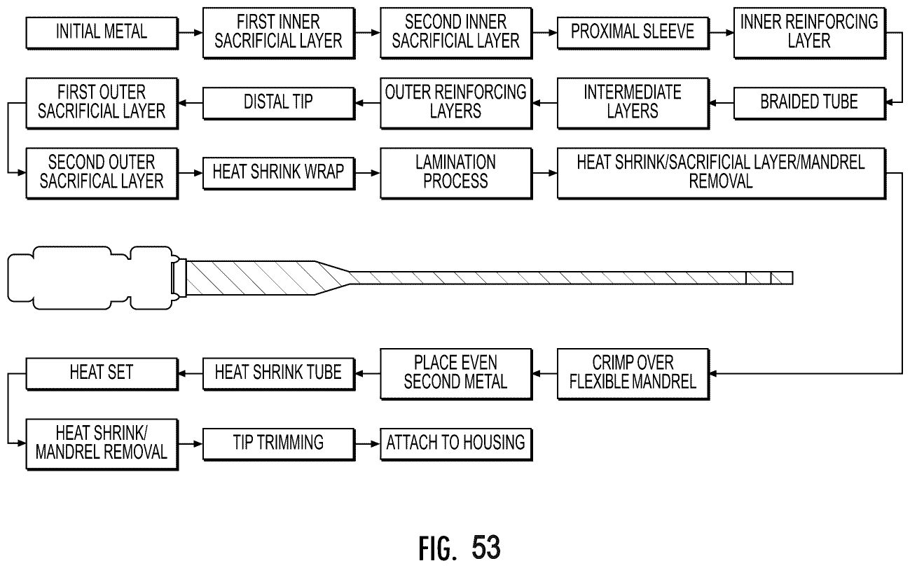

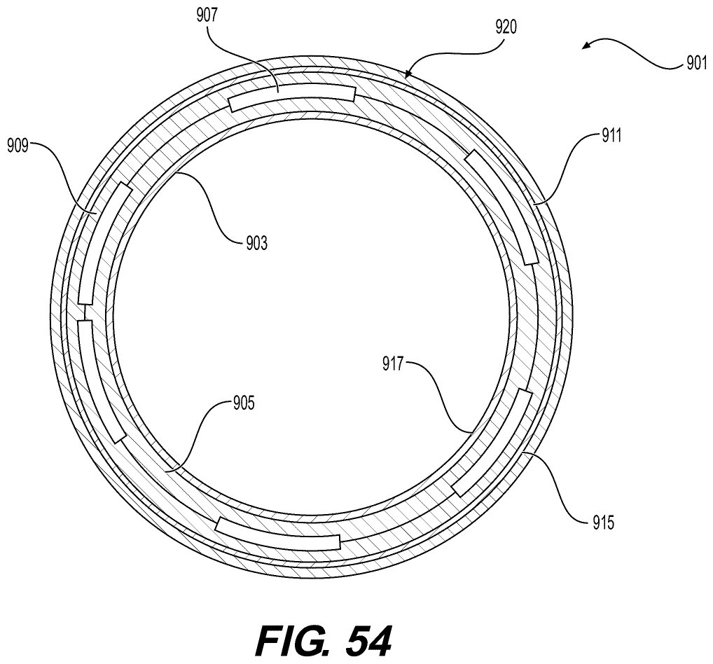

An example of a method of making an expandable sheath is depicted in FIG. 53. All materials mentioned in reference to FIG. 53 are exemplary only and may be substituted with other materials as detailed in this disclosure. In some implementations, the steps may take a different order. As a first step, an initial metal mandrel (e.g., stainless steel) can be wrapped with a first sacrificial preliminary layer (e.g., ePTFE), optionally followed by a second sacrificial preliminary layer (e.g., PTFE). The diameter of the initial metal mandrel plus the sacrificial layers will correspond approximately to the inner diameter of the expanded sheath. The sacrificial layers do not adhere to the mandrel or the subsequent sheath layers, thereby assisting with the ultimate removal of the sheath layers from the initial metal mandrel.

Next, in reference to FIG. 53, a proximal sleeve is positioned over a proximal end of the second sacrificial preliminary layer. A low friction inner liner comprising a first material such as Dyneema® is then wrapped or otherwise positioned around the second sacrificial preliminary layer and the distal end of the proximal sleeve. It is understood that the first material can be wrapped multiple times to form more than one polymer layer, depending on the desired application. In some aspects, for example, the one shown in FIGS. 48-52, a first polymeric layer can be formed by wrapping (or otherwise positioning) a second material around the inner liner (not shown in FIG. 49). The first polymeric layer can actually include several sublayers formed by wrapping the second material around multiple times. A braided layer (e.g., nitinol) is then positioned over the inner liner or the first polymeric layer. A second polymeric layer is then formed by wrapping a third material around the braided layer. Again, the second polymeric layer can include several sublayers, each formed by wrapping the third material multiple times. It is understood that in certain aspects, additional intermediate layers and sublayers can be present. In such aspects, these intermediate layers and sublayers (such as for example, silicone or elastomeric bands, additional polymeric layers, and/or radiopaque layers) are wrapped or otherwise positioned over the braided layer or over the second polymeric layer depending on the desired application. A low friction outer liner is formed by wrapping a fourth material (e.g., Dyneema®) or otherwise positioning the fourth material over the second polymeric layer or any additional intermediate layers if present. The low friction outer liner can also include two or more layers formed by wrapping the fourth material around multiple times.

Continuing to refer to FIG. 53, in the next step, a distal tip (e.g., LDPE) is positioned over the distal end of the low friction outer liner. Sacrificial outer layers such as ePTFE, PTFE, TPE, and/or PVC tapes can also be wrapped or otherwise positioned over the reinforcing layer and the distal tip, optionally followed by an additional outer sacrificial layer (e.g., ePTFE). These sacrificial layers are used to protect integrity of the sheath during the manufacturing process and are removed from the final product.

The layers are then covered by a heat shrink wrap and laminated by heating. This step allows the polymeric layers to flow into the unit cells of the braided layer. The sacrificial outer layers do not adhere to the sheath layers or the heat shrink wrap, thus assisting with the removal of the heat shrink wrap.

Continuing to refer to FIG. 53, in the next step, the layers are removed from the stainless steel mandrel, and the heat shrink wrap and sacrificial outer and sacrificial preliminary layers are removed from the sheath layers. The sheath layers are placed over a flexible mandrel (e.g., silicone) and crimped to create longitudinally-extending pleats. In some aspects, the partially-completed sheath is moved to a second metal mandrel with a diameter approximately the size of the inner diameter of the collapsed (unexpanded) sheath. A heat shrink tube/wrap is applied over the outside of the sheath, covering the longitudinally-extending pleats. The longitudinally-extending pleats are then set by an additional heating step, in which they at least partially bond to each other. The heat shrink tube is removed, and the nearly-completed sheath is removed from the second metal mandrel. The distal tip is trimmed, and the proximal housing is attached to finish the construction of the expandable sheath.

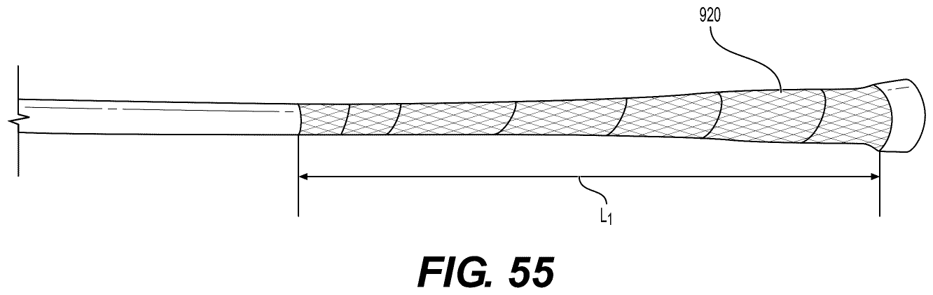

An example aspect of the disclosed sheath is shown in FIG. 54. In this aspect, a third polymeric layer 920 comprising one or more layers can be disposed on at least a portion of the proximal end of the sheath. A photograph of such an aspect is shown in FIG. 55, where L₁ shows a length of the third polymeric layer in the proximal portion. In such an aspect, a fifth material is wrapped radially outward of at least a portion of the outer liner 911 to form the third polymeric layer. In certain aspects, the fifth material is wrapped at least 2 times around the portion of the outer liner. Yet, in some implementations, the fifth material can be wrapped multiple times, forming from about 2 to about 10 layers.

In some implementations, the fifth material can be any material that is suitable for the desired application. Yet, in some implementations, the fifth material can comprise ultra-high molecular weight polyethylene (UHMWPE). In some implementations, the UHMWPE can be present as a fabric, a laminate, or a porous film or membrane. For example, the fifth material forming the third polymeric layer can comprise or be formed of Dyneema® UHMWPE. In some aspects, the third polymeric layer can comprise, or be formed of, the Dyneema Purity® membrane, which has a tensile strength of about 20 MPa.

In some exemplary and unlimiting aspects, the third polymeric layer can also be formed by coating. In such aspects, the UHMWPE can be provided as a polymeric solution, for example. Any known in the art coating methods can be utilized. For example, the coating methods can include dipping, doctor blade coating, spraying, and the like.

It is understood that in aspects where the third polymeric layer is present, such a layer is formed after the laminate structure described herein and comprising the inner liner, the first polymeric layer, the braided layer, the second polymeric layer, and the outer liner is formed.

In some implementations, after the third polymeric layer is formed either by wrapping the fifth material or by coating, a structure is heated to a temperature from about 120° C. to about 150° C., including exemplary values of about 125° C., about 130° C., about 135° C., about 140° C., and about 145° C. It is understood that at such temperatures, the third material is not fully melted and, therefore, is not expected to penetrate all the layers beneath it, and more specifically, it is not expected to be bound to the braided layer. In some implementations, at such temperatures, the third polymeric layer is at least partially bonded to the outer liner.

In certain aspects, prior to the exposure of the third polymeric layer to the described above temperature, a sacrificial heat tubing is first disposed on the third polymeric layer, and then the sheath is exposed to elevated temperatures. Yet, in some implementations, the heating of the third polymeric layer to form the described herein sheath can be performed without the presence of the sacrificial heath tubing.

In some implementations, where the third polymeric layer is laminated to at least a portion of the outer liner, an outer surface of the third polymeric layer is smoother than an outer surface of the outer liner anywhere on the sheath. In some implementations, an outer surface of the third polymeric layer exhibits less roughness than an outer surface of the outer liner anywhere on the sheath.

In yet some implementations, the third polymeric layer exhibits higher porosity than the outer liner.

In some implementations, the at least portion of the proximal end of the sheath that is covered by the third polymeric layer is up to about 15 cm, including exemplary values of about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, and about 14 cm.

In some implementations, at least a portion of the sheath with the third polymeric layer is inserted into a patient's body. In such aspects, the third polymeric sheath can form a substantial seal with the patient's natural anatomy to prevent unnecessary blood loss.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, specific conditions, etc.), but some errors and deviations should be accounted for.

The performance of the disclosed herein sheath was evaluated in an animal model with small bold vessels. Push and pull forces needed to operate the disclosed herein sheath were compared with the push and pull forces needed to operate other conventional and commercially available sheaths. The damage to the artery and the surrounding areas were evaluated.

The sheaths used herein comprised Dyneema@ inner and outer liners and cast polypropylene or low-density polyethylene as the first and the second polymeric layers. The sheath inner diameter ranged from 12 F to 14 F. The sheath was inserted either in the right or left femoral artery, and the results were compared.

Animals used in the study had an average weight of about 105-110 kg and had a femoral artery size of about 4.5-5.5 mm. It was found that disclosed herein, sheaths require significantly lower push force (25-55 N) when compared with a commercially available sheath having a different design (>60N).

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

EXAMPLE 1: An expandable sheath for deploying a medical device having a proximal end and a distal end, an inner surface and an outer surface and comprising: an inner liner comprising one or more polymer layers; wherein the inner liner has a first surface and an opposite second surface, wherein the first surface of the inner liner defines the inner surface of the sheath; a first polymeric layer surrounding radially outward of the inner liner, such that it positioned at the second surface of the inner liner and wherein the first polymeric layer comprises one or more sublayers; a braided layer disposed radially outward of the first polymeric layer; a second polymeric layer surrounding radially outward of the braided layer, wherein the second polymeric layer comprises one or more sublayers; an outer liner comprising one or more polymer layers; wherein the outer liner has a first surface and an opposite second surface, wherein the first surface of the outer liner overlies the second polymeric layer, and wherein the second surface of the liner layer defines the outer surface of the sheath; wherein the inner liner, the first polymeric layer, the second polymeric layer and the outer liner form a laminate structure; and wherein when a medical device is passed through the sheath, a diameter of the sheath locally expands from a first unexpended diameter around the medical device to a second expanded diameter, while the first and second polymeric layers resist axial elongation of the sheath such that a length of the sheath remains substantially constant; and wherein the sheath resiliently returns to a third diameter after the passage of the medical device.

EXAMPLE 2: The expandable sheath of any one of examples herein, particularly example 1, wherein the third diameter is substantially similar to the first unexpanded diameter.

EXAMPLE 3: The expandable sheath of any examples herein, particularly examples 1-2, wherein the inner liner comprises two or more polymer layers, wherein each of the polymer layers has a thickness from about 0.5 microns to about 40 microns.

EXAMPLE 4: The expandable sheath of any one of examples herein, particularly example 3, wherein the two or more polymer layers are laminated together.

EXAMPLE 5: The expandable sheath of any one of examples herein, particularly examples 1-4, wherein the outer liner comprises two or more polymer layers, wherein each of the polymer layers has a thickness from about 0.5 microns to about 40 microns.

EXAMPLE 6: The expandable sheath of any one of examples herein, particularly example 5, wherein the two or more polymer layers are laminated together.

EXAMPLE 7: The expandable sheath of any one of examples herein, particularly examples 1-6, wherein the first polymeric layer comprises two or more polymer sublayers, wherein each of the polymer sublayers has a thickness from about 0.5 microns to about 40 microns.

EXAMPLE 8: The expandable sheath of any one of examples herein, particularly example 7, wherein the two or more polymer sublayers are laminated together.

EXAMPLE 9: The expandable sheath of any one of examples herein, particularly examples 1-8, wherein the second polymeric layer comprises two or more polymer sublayers, wherein each of the polymeric layers has a thickness from about 0.5 microns to about 40 microns.

EXAMPLE 10: The expandable sheath of any one of examples herein, particularly example 9, wherein the two or more polymer sublayers are laminated together.

EXAMPLE 11: The expandable sheath of any one of examples herein, particularly examples 1-10, wherein the first polymeric layer is provided as a film or as a coating.

EXAMPLE 12: The expandable sheath of any one of examples herein, particularly example 11, wherein the one or more layers of the first polymeric layer are one or more layers of the film.

EXAMPLE 13: The expandable sheath of any one of examples herein, particularly example 12, wherein the one or more layers of the first polymeric layer are one or more layers of the coating.

EXAMPLE 14: The expandable sheath of any one of examples herein, particularly examples 1-13, wherein the second polymeric layer is provided as a film or as a coating.

EXAMPLE 15: The expandable sheath of any one of examples herein, particularly example 14, wherein the one or more layers of the second polymeric layer are one or more layers of the film.

EXAMPLE 16: The expandable sheath of any one of examples herein, particularly example 14, wherein the one or more layers of the second polymeric layer are one or more layers of the coating.

EXAMPLE 17: The expandable sheath of any one of examples herein, particularly examples 1-16, wherein a portion of the proximal end of the expandable sheath further comprises a third polymeric layer comprising one or more layers and surrounding radially outward of the outer layer.

EXAMPLE 18: The expandable sheath of any one of examples herein, particularly example 17, wherein the third polymeric layer is provided as a film or as a coating.

EXAMPLE 19: The expandable sheath of any one of examples herein, particularly examples 17 or 18, wherein the third polymeric layer is applied to the outer liner, after the inner liner, the first polymeric layer, the second polymeric layer, and the outer liner form the laminate structure.

EXAMPLE 20: The expandable sheath of any one of examples herein, particularly examples 17-19, wherein an outer surface of the third polymeric layer is substantially smoother than an outer surface of the outer liner.

EXAMPLE 21: The expandable sheath of any one of examples herein, particularly examples 17-20, wherein an outer surface of the third polymeric layer has a roughness substantially smaller than an outer surface of the outer liner.

EXAMPLE 22: The expandable sheath of any one of examples herein, particularly examples 17-21, wherein the third polymeric layer has a porosity substantially larger than a porosity of the outer liner.

EXAMPLE 23: The expandable sheath of any one of examples herein, particularly examples 17-22, wherein the third polymeric layer is bonded to at least a portion of the outer liner.

EXAMPLE 24: The expandable sheath of any one of examples herein, particularly examples 17-23, wherein the third polymeric layer is not bonded to the braided layer.

EXAMPLE 25: The expandable sheath of any one of examples herein, particularly examples 17-24, wherein the portion of the proximal end is from about 10 mm to about 150 mm from a proximal edge of the sheath.

EXAMPLE 26: The expandable sheath of any one of examples herein, particularly examples 17-25, wherein the third polymeric layer comprises from about 2 to about 10 layers.

EXAMPLE 27: The expandable sheath of any one of examples herein, particularly examples 17-26, wherein the third polymeric layer forms a substantial seal with a patient's natural anatomy upon insertion into a patient's body.

EXAMPLE 28: The expandable sheath of any one of examples herein, particularly examples 1-27, wherein the one or more polymer layers of the inner liner and/or outer liner comprise an ultra-high-molecular-weight polyethylene (UHMWP) polymer layer.

EXAMPLE 29: The expandable sheath of any one of examples herein, particularly example 28, wherein the UHMWP polymer layer is a porous film.

EXAMPLE 30: The expandable sheath of any one of examples herein, particularly example 28 or 29, wherein the UHMWP polymer is Dyneema®.

EXAMPLE 31: The expandable sheath of any one of examples herein, particularly examples 1-30, wherein the first polymeric layer comprises at least one sublayer comprising a polyolefin or a polyurethane.

EXAMPLE 32: The expandable sheath of any one of examples herein, particularly examples 1-31, wherein the second polymeric layer comprises at least one sublayer comprising a polyolefin or a polyurethane.

EXAMPLE 33: The expandable sheath of any one of examples herein, particularly example 31 or 32, wherein the polyolefin comprises polyethylene, polypropylene, or a combination thereof.

EXAMPLE 34: The expandable sheath of any one of examples herein, particularly example 33, wherein the polypropylene comprises a bi-oriented polypropylene, a cast polypropylene, or a combination thereof.

EXAMPLE 35: The expandable sheath of any one of examples herein, particularly example 33, wherein the polyethylene comprises a low-density polyethylene (LDPE), a high density polyethylene (HDPE), or a combination thereof.

EXAMPLE 36: The expandable sheath of any one of examples herein, particularly example 33, wherein the polyolefin comprises a bi-oriented polypropylene, a cast polypropylene, a low-density polyethylene (LDPE), a high density polyethylene (HDPE), or a combination thereof.

EXAMPLE 37: The expandable sheath of any one of examples herein, particularly example 32, wherein the polyurethane comprises a thermoplastic polyurethane.

EXAMPLE 38: The expandable sheath of any one of examples herein, particularly examples 17-37, wherein the third polymeric layer comprises an ultra-high-molecular-weight polyethylene (UHMWP) polymer layer.

EXAMPLE 39: The expandable sheath of any one of examples herein, particularly example 38, wherein the UHMWP polymer layer is a porous film.

EXAMPLE 40: The expandable sheath of any one of examples herein, particularly example 38 or 39, wherein the UHMWP polymer is Dyneema®.

EXAMPLE 41: The expandable sheath of any one of examples herein, particularly examples 1-40, wherein the tensile strength of the first polymeric layer and/or the second polymeric layer is substantially the same or different from the tensile strength of the inner liner and/or outer liner.

EXAMPLE 42: The expandable sheath of any one of examples herein, particularly examples 1-41, wherein the tensile strength of the first polymeric layer and/or the second polymeric layer is larger than the tensile strength of the inner liner and/or outer liner.

EXAMPLE 43: The expandable sheath of any one of examples herein, particularly examples 1-41, wherein the tensile strength of the first polymeric layer and/or the second polymeric is smaller than the tensile strength of the inner liner and/or outer liner.

EXAMPLE 44: The expandable sheath of any one of examples herein, particularly examples 1-43, wherein the inner surface of the sheath is substantially smooth.

EXAMPLE 45: The expandable sheath of any one of examples herein, particularly examples 1-44, wherein the sheath comprises a plurality of longitudinally extending pleats.

EXAMPLE 46: The expandable sheath of any one of examples herein, particularly example 45, wherein the plurality of pleats extend around at least a portion of a circumference of the sheath.

EXAMPLE 47: The expandable sheath of any one of examples herein, particularly example 45 or 46, wherein each of the plurality of pleats comprises at least a portion of the inner liner, at least a portion of the first polymeric layer, at least a portion of the second polymeric layer, and at least a portion of the outer liner.

EXAMPLE 48: The expandable sheath of any one of examples herein, particularly examples 45-47, wherein the plurality of pleats extend along at least a portion of a length of the sheath.

EXAMPLE 49: The expandable sheath of any one of examples herein, particularly examples 45-48, wherein the plurality of pleats are uniformly distributed along at least a portion of a length of the sheath and at least a portion of a circumference of the sheath.

EXAMPLE 50: The expandable sheath of any one of examples herein, particularly examples 45-49, wherein the plurality of pleats are randomly distributed along at least a portion of a length of the sheath and at least a portion of a circumference of the sheath.

EXAMPLE 51: The expandable sheath of any one of examples herein, particularly examples 45-50, wherein the plurality of pleats form a plurality of circumferentially spaced ridges and a plurality of circumferentially spaced valleys, and wherein, as the medical device is passed through the sheath, the ridges and valleys at least partially level out to allow a sheath wall to radially expand.

EXAMPLE 52: The expandable sheath of any one of examples herein, particularly examples 1-51, wherein the first polymeric layer is bonded or adhered to at least a portion of the second surface of the inner liner, and the second polymeric layer is bonded or adhered to at least a portion of the first surface of the outer liner.

EXAMPLE 53: The expandable sheath of any one of examples herein, particularly example 52, when the one or more polymer layers of the inner liner comprise the porous film, at least a portion of the first polymeric layer extends within at least a portion of the porous film of the inner liner.

EXAMPLE 54: The expandable sheath of any one of examples herein, particularly example 52 or 53, when the one or more polymer layers of the outer liner comprises the porous film, at least a portion of the second polymeric layer extends within at least a portion of the porous film of the outer liner.

EXAMPLE 55: The expandable sheath of any one of examples herein, particularly examples 52-54, wherein the inner liner and the outer liner exhibit a mechanical strength higher than a mechanical strength of a substantially identical reference sheath in the absence of the laminate structure.

EXAMPLE 56: The expandable sheath of any one of examples herein, particularly examples 1-55, wherein the braided layer comprises a plurality of helical multifilar filaments braided together.

EXAMPLE 57: The expandable sheath of any one of examples herein, particularly example 56, wherein the plurality of helical multifilar filaments comprise nitinol.

EXAMPLE 58: The expandable sheath of any one of examples herein, particularly example 56 or 57, wherein the braided layer has a proximal end and a distal end.

EXAMPLE 59: The expandable sheath of any one of examples herein, particularly example 58, wherein the braided layer comprises a plurality of closed loops at the distal end of the braid.

EXAMPLE 60: The expandable sheath of any one of examples herein, particularly examples 58-59, wherein the proximal end of the braided layer is positioned along the proximal end of the sheath.

EXAMPLE 61: The expandable sheath of any one of examples herein, particularly examples 58-60, wherein the braided layer has a length extending from the proximal end of the braided layer to the distal end of the braided layer and wherein the braided layer length is shorter than a length of the sheath measured from proximal end of the sheath to the distal end of the sheath.

EXAMPLE 62: The expandable sheath of any one of examples herein, particularly examples 56-61, wherein the braided layer has a weave pattern of 1×1.

EXAMPLE 63: The expandable sheath of any one of examples herein, particularly examples 56-61, wherein the braided layer has a weave pattern of 2×2.

EXAMPLE 64: The expandable sheath of any one of examples herein, particularly examples 56-61, wherein the braided layer has a weave pattern of 2×1.

EXAMPLE 65: The expandable sheath of any one of examples herein, particularly examples 56-64, wherein the braided layer comprises a self-contracting material.

EXAMPLE 66: The expandable sheath of any one of examples herein, particularly examples 56-65, wherein the braided layer comprises a shape memory material exhibiting superelastic properties at temperatures at or above 15 degrees Celsius.

EXAMPLE 67: The expandable sheath of any one of examples herein, particularly examples 56-66, wherein a portion of the plurality of filaments comprises an elastic coating.

EXAMPLE 68: The expandable sheath of any one of examples herein, particularly examples 56-67, wherein the filaments of the braided layer are movable between the first and second polymeric layers such that the braided layer is configured to radially expand as a medical device is passed through the sheath while the length of the sheath remains substantially constant.

EXAMPLE 69: The expandable sheath of any one of examples herein, particularly examples 56-68, wherein the filaments of the braided layer are resiliently buckled when the sheath is in a collapsed configuration, and the first and second polymeric layers are attached to each other at a plurality of open spaces between the filaments of the braided layer.

EXAMPLE 70: The expandable sheath of any one of examples herein, particularly examples 1-69, further comprising an outer cover formed of a heat shrink material and extending over at least a longitudinal portion of the first polymeric layer and the second polymeric layer, the outer cover comprising one or more longitudinally extending slits, weakened portions, or scorelines.

EXAMPLE 71: The expandable sheath of any one of examples herein, particularly examples 1-70, wherein the sheath further comprises an elastic outer layer that applies an inward radial force on a sheath wall, biasing the sheath toward the unexpanded state.

EXAMPLE 72: The expandable sheath of any one of examples herein, particularly examples 1-71, wherein the sheath exhibits an improved column strength when compared with a substantially identical reference sheath in the absence of a laminate structure.

EXAMPLE 73: The expandable sheath of any one of examples herein, particularly examples 1-72, wherein the medical device is a prosthetic heart valve.

EXAMPLE 74: An expandable sheath for deploying a medical device having a proximal end and a distal end, an inner surface and an outer surface and comprising: an inner liner comprising one or more polymer layers; wherein the inner liner has a first surface and an opposite second surface, wherein the first surface of the inner liner defines the inner surface of the sheath; a first polymeric layer surrounding radially outward of the inner liner, such that it positioned at the second surface of the inner liner and wherein the first polymeric layer comprises one or more sublayers; a braided layer disposed radially outward of the first polymeric layer; a second polymeric layer surrounding radially outward of the braided layer, wherein the second polymeric layer comprises one or more sublayers; an outer liner comprising one or more polymer layers; wherein the outer liner has a first surface and an opposite second surface, wherein the first surface of the outer liner overlies the second polymeric layer, and wherein the second surface of the liner layer defines the outer surface of the sheath; wherein the inner liner, the first polymeric layer, the second polymeric layer and the outer liner form a laminate structure; wherein the inner liner, the first polymeric layer, the second polymeric layer and the outer liner form a laminate structure; and wherein when a medical device is passed through the sheath, the diameter of the sheath locally expands from a first unexpended diameter around the medical device to a second expanded diameter, while the first and second polymeric layers resist axial elongation of the sheath such that a length of the sheath remains substantially constant; and wherein the sheath resiliently returns to a third diameter after the passage of the medical device.

EXAMPLE 75: A method of making an expandable sheath, the method comprising: forming an inner liner; wherein the inner liner comprises one or more polymer layers and wherein the inner liner has a first surface and an opposite second surface; forming a first polymeric layer, wherein the first polymeric layer is positioned radially outward of the inner liner; wherein the first polymeric layer comprises one or more sublayers; and wherein the first polymeric layer overlies the second surface of the inner liner; positioning a braided layer radially outward of a first polymeric layer; forming a second polymeric layer such that it is positioned radially outward of the braided layer; wherein the second polymeric layer comprises one or more sublayers; forming an outer liner radially outward of the second polymeric layer; wherein the outer liner comprises one or more polymer layers; and wherein the outer liner has a first surface and an opposite second surface and wherein the first surface of the outer liner is in contact with at least a portion of the second polymeric layer; heating the inner liner, the first polymeric layer, the braided layer, the second polymeric layer, and the outer liner to form a laminate structure; and crimping the laminate structure to form a plurality of longitudinally-extending pleats, wherein the plurality of longitudinally-extending pleats are configured to expand upon passage of a medical device through the sheath.

EXAMPLE 76: The method of any one of examples herein, particularly example 75, wherein the step of forming the inner liner comprises wrapping a first material around an initial mandrel.

EXAMPLE 77: The method of any one of examples herein, particularly example 75 or 76, wherein the first material comprises an ultra-high-molecular-weight polyethylene (UHMWP) polymer film.

EXAMPLE 78: The method of any one of examples herein, particularly example 76 or 77, wherein the step of wrapping of the first material comprises forming two or more polymer layers of the inner liner.

EXAMPLE 79: The method of any one of examples herein, particularly example 77 or 78, wherein the UHMWP polymer film is porous.

EXAMPLE 80: The method of any one of examples herein, particularly examples 75-79, wherein the step of forming the first polymeric layer comprises wrapping a second material radially outward of the second surface of the inner liner.

EXAMPLE 81: The method of any one of examples herein, particularly example 80, wherein the step of wrapping of the second material comprises forming two or more polymer sublayers of the first polymeric layer.

EXAMPLE 82: The method of any one of examples herein, particularly examples 75-81, wherein the step of forming the first polymeric layer comprises coating of the second surface of the inner liner with one or more layers of a second material to form the one or more sublayers of the first polymeric layer.

EXAMPLE 83: The method of any one of examples herein, particularly example 82, wherein the coating comprises dipping, spray-coating, brush-coating, doctor blade coating, or any combination thereof.

EXAMPLE 84: The method of any one of examples herein, particularly examples 75-83, wherein the step of forming the second polymeric layer comprises wrapping a third material outward of the braided layer.

EXAMPLE 85: The method of any one of examples herein, particularly example 84, wherein the step of wrapping of the third material comprises forming two or more polymer sublayers of the second polymeric material.

EXAMPLE 86: The method of any one of examples herein, particularly examples 75-83, wherein the step of forming the second polymeric layer comprises coating the braided layer with one or more layers of a third material to form the one or more sublayers of the first polymeric layer.

EXAMPLE 87: The method of any one of examples herein, particularly example 86, wherein the coating comprises dipping, spray-coating, brush-coating, doctor blade coating, or any combination thereof.

EXAMPLE 88: The method of any one of examples herein, particularly examples 81-87, wherein the second material and the third material are the same or different.

EXAMPLE 89: The method of any one of examples herein, particularly examples 81-88, wherein the second material and/or the third material comprise a polyolefin or a polyurethane.

EXAMPLE 90: The method of any one of examples herein, particularly example 89, wherein the polyolefin comprises polyethylene, polypropylene, or a combination thereof.

EXAMPLE 91: The method of any one of examples herein, particularly example 90, wherein the polypropylene comprises a bi-oriented polypropylene, a cast polypropylene, or a combination thereof.

EXAMPLE 92: The method of any one of examples herein, particularly example 91, wherein the polyethylene comprises a low-density polyethylene (LDPE), a high density polyethylene (HDPE), or a combination thereof.

EXAMPLE 93: The method of any one of examples herein, particularly example 92, wherein the polyolefin comprises a bi-oriented polypropylene, a cast polypropylene, a low-density polyethylene (LDPE), a high density polyethylene (HDPE), or a combination thereof.

EXAMPLE 94: The method of any one of examples herein, particularly example 89, the second material and/or the third material comprise a thermoplastic polyurethane.

EXAMPLE 95: The method of any one of examples herein, particularly examples 75-94, wherein the step of forming the outer liner comprises wrapping a fourth material radially outward of the second surface of the second polymeric layer.

EXAMPLE 96: The method of any one of examples herein, particularly example 95, wherein the step of wrapping the fourth material comprises forming two or more polymer layers of the outer layer.

EXAMPLE 97: The method of any one of examples herein, particularly example 95 or 96, wherein the fourth material and the first material are the same or different.

EXAMPLE 98: The method of any one of examples herein, particularly examples 95-97, wherein the fourth material comprises an ultra-high-molecular-weight polyethylene (UHMWP) polymer film.

EXAMPLE 99: The method of any one of examples herein, particularly example 98, wherein the UHMWP polymer film is porous.

EXAMPLE 100: The method of any one of examples herein, particularly examples 75-99, wherein after forming the laminate structure, the method further comprises forming a third polymeric layer at at least a portion of the proximal end of the sheath.

EXAMPLE 101: The method of any one of examples herein, particularly example 100, the step of forming the third polymeric layer comprises wrapping a fifth material radially outward of an outer surface of the outer liner, forming one or more polymer layers of the third polymeric layer.

EXAMPLE 102: The method of any one of examples herein, particularly example 101, wherein the fifth material comprises an ultra-high-molecular-weight polyethylene (UHMWP) polymer film.

EXAMPLE 103: The method of any one of examples herein, particularly example 102, wherein the UHMWP polymer film is porous.

EXAMPLE 104: The method of any one of examples herein, particularly examples 100-103, wherein the sheath is heated to a temperature from about 120° C. to about 150° C. to laminate the third polymeric layer to at least a portion of the outer liner.

EXAMPLE 105: The method of any one of examples herein, particularly examples 100-104, wherein an outer surface of the third polymeric layer is substantially smoother than an outer surface of the outer liner.

EXAMPLE 106: The method of any one of examples herein, particularly examples 100-105, wherein an outer surface of the third polymeric layer has a roughness substantially smaller than an outer surface of the outer liner.

EXAMPLE 107: The method of any one of examples herein, particularly examples 100-106, wherein the third polymeric layer has a porosity substantially larger than a porosity of the outer liner.

EXAMPLE 108: The method of any one of examples herein, particularly examples 100-107, wherein the third polymeric layer is not bonded to the braided layer.

EXAMPLE 109: The method of any one of examples herein, particularly examples 100-108, wherein the portion of the proximal end is from about 10 mm to about 150 mm from a proximal edge of the sheath.

EXAMPLE 110: The method of any one of examples herein, particularly examples 100-109, wherein the third polymeric layer comprises from about 2 to about 10 layers.

EXAMPLE 111: The method of any one of examples herein, particularly examples 100-110, wherein the third polymeric layer forms a substantial seal with a patient's natural anatomy upon insertion into a patient's body.

EXAMPLE 112: The method of any one of examples herein, particularly examples 75-111, wherein the tensile strength of the first polymeric layer and/or the second polymeric layer is substantially the same or different as the tensile strength of the inner liner and/or outer liner.

EXAMPLE 113: The method of any one of examples herein, particularly examples 75-112, wherein the tensile strength of the first polymeric layer and/or the second polymeric layer is larger than the tensile strength of the inner liner and/or outer liner.

EXAMPLE 114: The method of any one of examples herein, particularly examples 75-112, wherein the tensile strength of the first polymeric layer and/or the second polymeric is smaller than the tensile strength of the inner liner and/or outer liner.

EXAMPLE 115: The method of any one of examples herein, particularly examples 75-114, wherein prior to the step of forming the laminate structure, the method further comprises positioning a first heat shrink tube radially outward of the second surface of the outer liner.

EXAMPLE 116: The method of any one of examples herein, particularly example 115, wherein the step of heating comprises applying heat to the inner liner, the first polymeric layer, the braided layer, the second polymeric layer, the outer liner, and the heat shrink tube.

EXAMPLE 117: The method of any one of examples herein, particularly example 116, further comprises removing the first heat shrink tube after forming the laminate structure.

EXAMPLE 118: The method of any one of examples herein, particularly examples 75-117, wherein after the step of heating, the method comprises removing the laminate structure from the initial mandrel prior.

EXAMPLE 119: The method of any one of examples herein, particularly examples 75-118 wherein the step of crimping comprises positioning the laminate structure on a second mandrel, wherein the second mandrel is the same or different from the first mandrel and crimping the laminate structure to form the plurality of longitudinally-extending pleats.

EXAMPLE 120: The method of any one of examples herein, particularly example 119, further comprising positioning a second heat shrink tube radially outward of the plurality of longitudinally-extending pleats and heating the laminate structure having the plurality of longitudinally-extending pleats and the second heat shrink tube to cause the plurality of longitudinally-extending pleats to at least partially bond to each other.

EXAMPLE 121: The method of any one of examples herein, particularly example 120, further comprising removing the second heat shrink tube.

EXAMPLE 122: The method of any one of examples herein, particularly examples 75-121, wherein the step of heating comprises at least partially bonding the inner liner, the first polymeric layer, the second polymer layer, and the outer liner together.

EXAMPLE 123: The method of any one of examples herein, particularly examples 75-122, wherein the step of heating comprises at least partially encapsulating the braided layer within the inner liner, the first polymeric layer, the second polymer layer, and the outer liner.

EXAMPLE 124: The method of any one of examples herein, particularly examples 80-123, wherein the step of heating comprises the second material at least partially penetrating the porous first material.

EXAMPLE 125: The method of any one of examples herein, particularly examples 84-124, wherein the step of heating comprises the third material at least partially penetrating the porous fourth material.

EXAMPLE 126: The method of any one of examples herein, particularly examples 75-125, further comprising positioning one or more inner sacrificial layers prior to forming the inner liner and removing the one or more inner sacrificial layers prior to creating the plurality of longitudinally-extending pleats.

EXAMPLE 127: The method of any one of examples herein, particularly examples 75-126, further comprising positioning one or more outer sacrificial layers radially outward of the outer liner after forming the outer liner and removing the one or more outer sacrificial layers prior to creating the plurality of longitudinally-extending pleats.

EXAMPLE 128: The method of any one of examples herein, particularly examples 75-127, further comprising positioning a cushioning layer adjacent to the braided layer and removing it in a later processing step.

EXAMPLE 129: The method of any one of examples herein, particularly example 128, further comprising applying a sealing layer to the cushioning layer.

EXAMPLE 130: The method of any one of examples herein, particularly examples 75-129, wherein the braided layer comprises a plurality of helical multifilar filaments braided together.

EXAMPLE 131: The method of any one of examples herein, particularly example 130, wherein the plurality of helical multifilar filaments comprise nitinol.

EXAMPLE 132: The method of any one of examples herein, particularly example 130 or 131, wherein the method comprises applying an elastic coating to a portion of the plurality of helical multifilar filaments

EXAMPLE 133: The method of any one of examples herein, particularly examples 75-132, further comprising setting the braided layer to a contracted diameter prior to placing the braided layer radially outward of the first polymeric layer.

EXAMPLE 134: The method of any one of examples herein, particularly examples 75-133, wherein when a medical device is passed through the formed sheath, the diameter of the sheath locally expands from a first unexpended diameter around the medical device to a second expanded diameter, while the first and second polymeric layers resist axial elongation of the sheath such that a length of the sheath remains substantially constant; and wherein the sheath resiliently returns to a third diameter after the passage of the medical device.

EXAMPLE 135: A method of making an expandable sheath, the method comprising: forming an inner liner; wherein the inner liner comprises one or more polymer layers and wherein the inner liner has a first surface and an opposite second surface; forming a first polymeric layer, wherein the first polymeric layer is positioned radially outward of the inner liner; wherein the first polymeric layer comprises one or more sublayers; and wherein the first polymeric layer overlies the second surface of the inner liner; positioning a braided layer radially outward of a first polymeric layer; forming a second polymeric layer such that it is positioned radially outward of the braided layer; wherein the second polymeric layer comprises one or more sublayers; forming an outer liner radially outward of the second polymeric layer; wherein the outer liner comprises one or more polymer layers, and wherein the outer liner has a first surface and an opposite second surface, and wherein the first surface of the outer liner is in contact with at least a portion of the second polymeric layer; heating the inner liner, the first polymeric layer, the braided layer, the second polymeric layer, and the outer liner to form a laminate structure; and crimping the laminate structure to form a plurality of longitudinally-extending pleats, wherein the plurality of longitudinally-extending pleats are configured to expand upon passage of a medical device through the sheath; and then forming a third polymeric layer radially outward of at least a portion of the outer liner; wherein the third polymeric layer comprises one or more polymer layers.

EXAMPLE 136: A method of delivering a prosthetic device to a procedure site, the method comprising: inserting an expandable sheath at least partially into the vasculature of the patient, the expandable sheath comprising a plurality of radially arranged layers including an inner liner, a first polymeric layer radially outward of the inner liner, a braided layer radially outward of the first polymeric layer, a second polymeric layer radially outward of the braided layer, and an outer liner, and wherein the sheath comprises a plurality of longitudinally-extending pleats; advancing a medical device through an inner lumen defined by a first surface of the inner liner of the sheath, the medical device applying an outward radial force on the inner liner of the sheath; locally expanding the sheath from an unexpanded state to a locally expanded state; at least partially unfolding the plurality of longitudinally-extending pleats during a local expansion of the sheath, wherein each of the plurality of longitudinally-extending pleats incorporates at least a portion of the plurality of radially arranged layers; locally collapsing the sheath from the locally expanded state at least partially back to the unexpanded state after passage of the medical device.

EXAMPLE 137 A method of delivering a prosthetic device to a procedure site, the method comprising: inserting an expandable sheath at least partially into the vasculature of the patient, the expandable sheath comprising a plurality of radially arranged layers including an inner liner, a first polymeric layer radially outward of the inner liner, a braided layer radially outward of the first polymeric layer, a second polymeric layer radially outward of the braided layer, an outer liner, and a third polymer layer disposed radially outward of at least a portion of the outer liner, and wherein the sheath comprises a plurality of longitudinally-extending pleats; advancing a medical device through an inner lumen defined by a first surface of the inner liner of the sheath, the medical device applying an outward radial force on the inner liner of the sheath; locally expanding the sheath from an unexpanded state to a locally expanded state; at least partially unfolding the plurality of longitudinally-extending pleats during a local expansion of the sheath, wherein each of the plurality of longitudinally-extending pleats incorporates at least a portion of the plurality of radially arranged layers; and locally collapsing the sheath from the locally expanded state at least partially back to the unexpanded state after passage of the medical device.

EXAMPLE 138: The method of delivering a prosthetic device of any one of examples herein, particularly example 136 or 137, wherein locally expanding the sheath from the unexpanded state to the locally expanded state comprises at least partially straightening a plurality of filaments of the braided layer.

EXAMPLE 139: The method of delivering a prosthetic device of any one of examples herein, particularly examples 136-138, wherein locally expanding the sheath comprises at least partially smoothing the plurality of longitudinally-extending pleats.

EXAMPLE 140: The method of delivering a prosthetic device of any one of examples herein, particularly examples 136-139, wherein locally collapsing the sheath comprises directing an inward radial force on the plurality of radially arranged layers.

EXAMPLE 141: The method of delivering a prosthetic device of any one of examples herein, particularly example 140, wherein directing an inward radial force comprises compressing the plurality of radially arranged layers with a tubular outer elastic layer.

EXAMPLE 142: The method of delivering a prosthetic device of any one of examples herein, particularly example 140 or 141, wherein directing an inward radial force on the plurality of radially arranged layers comprises coupling movement of the inner liner, the first polymeric layer, the second polymeric layer, and the outer liner with movement of the braided layer, wherein the braided layer comprises a self-contracting material.

EXAMPLE 143: The method of delivering a prosthetic device of any one of examples herein, particularly examples 136-142, wherein the step of locally collapsing the sheath comprises buckling a plurality of filaments of the braided layer.

EXAMPLE 144: The method of delivering a prosthetic device of any one of examples herein, particularly examples 136-143, wherein advancing the medical device comprises advancing a prosthetic heart valve through an inner lumen defined by a first innermost surface of the inner liner of the sheath.

EXAMPLE 145: The method of delivering a prosthetic device of any one of examples herein, particularly example 144, further comprising introducing the prosthetic heart valve to the procedure site and expanding the prosthetic heart valve within the procedure site.

Throughout this application, various publications and patent applications are referenced. The disclosures of these publications in their entirety are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein but which conflicts with existing definitions, statements, or other disclosure material set forth herein will only be incorporated to the extent that no conflict arises between that incorporated material and the existing disclosure material.

1. An expandable sheath for deploying a medical device, the expandable sheath comprising:
an inner liner comprising one or more polymer layers; wherein the inner liner has a first surface and an opposite second surface, wherein the first surface of the inner liner defines an inner surface of the expandable sheath;
a first polymeric layer surrounding radially outward of the inner liner, such that it is positioned at the second surface of the inner liner and wherein the first polymeric layer comprises one or more sublayers;
a braided layer disposed radially outward of the first polymeric layer;
a second polymeric layer surrounding radially outward of the braided layer, wherein the second polymeric layer comprises one or more sublayers;
an outer liner comprising one or more polymer layers; wherein the outer liner has a first surface and an opposite second surface, wherein the first surface of the outer liner overlies the second polymeric layer, and wherein the second surface of the outer liner defines an outer surface of the expandable sheath;
wherein the inner liner, the first polymeric layer, the second polymeric layer, and the outer liner form a laminate structure; and
wherein, when a medical device is passed through the sheath, a diameter of the sheath locally expands from a first unexpended diameter around the medical device to a second expanded diameter, while the first and second polymeric layers resist axial elongation of the sheath such that a length of the sheath remains substantially constant; and
wherein the sheath resiliently returns to a third diameter after the passage of the medical device.

2. The expandable sheath of claim 1, wherein the inner liner comprises two or more polymer layers, wherein each of the polymer layers has a thickness from about 0.5 microns to about 40 microns, and wherein the two or more polymer layers are laminated together and/or wherein the outer liner comprises two or more polymer layers, wherein each of the polymer layers has a thickness from about 0.5 microns to about 40 microns, and wherein the two or more polymer layers are laminated together.

3. The expandable sheath of claim 1, wherein the first polymeric layer comprises two or more polymer sublayers, wherein each of the polymer sublayers has a thickness from about 0.5 microns to about 40 microns, and wherein the two or more polymer sublayers are laminated together and/or wherein the second polymeric layer comprises two or more polymer sublayers, wherein each of the polymeric layers has a thickness from about 0.5 microns to about 40 microns, and wherein the two or more polymer sublayers are laminated together.

4. The expandable sheath of claim 1, wherein a portion of a proximal end of the expandable sheath further comprises a third polymeric layer comprising one or more layers and surrounding radially outward of the outer liner such that the third polymeric layer is bonded to at least a portion of the outer liner, and wherein the third polymeric layer forms a substantial seal with a patient's natural anatomy upon insertion into a patient's body; and wherein the portion of the proximal end is from about 10 mm to about 150 mm from a proximal edge of the sheath.

5. The expandable sheath of claim 4, wherein the third polymeric layer is applied to the outer liner, after the inner liner, the first polymeric layer, the second polymeric layer, and the outer liner form the laminate structure.

6. The expandable sheath of claim 1, wherein the one or more polymer layers of the inner liner and/or outer liner comprises an ultra-high-molecular-weight polyethylene (UHMWP) polymer layer.

7. The expandable sheath of claim 1, wherein the first polymeric layer comprises at least one sublayer comprising a polyolefin or a polyurethane, and/or wherein the second polymeric layer comprises at least one sublayer comprising a polyolefin or a polyurethane.

8. The expandable sheath of claim 7, wherein the polyolefin comprises a polypropylene comprising a bi-oriented polypropylene, a cast polypropylene, or a combination thereof and/or wherein the polyolefin comprises a polyethylene comprising a low-density polyethylene (LDPE), a high density polyethylene (HDPE), or a combination thereof.

9. The expandable sheath of claim 4, wherein the third polymeric layer comprises an ultra-high-molecular-weight polyethylene (UHMWP) polymer layer.

10. The expandable sheath of claim 1, wherein the sheath comprises a plurality of longitudinally extending pleats, wherein the plurality of pleats extend around at least a portion of a circumference of the sheath, and wherein each of the plurality of pleats comprises at least a portion of the inner liner, at least a portion of the first polymeric layer, at least a portion of the second polymeric layer, and at least a portion of the outer liner.

11. The expandable sheath of claim 10, wherein the plurality of pleats form a plurality of circumferentially spaced ridges and a plurality of circumferentially spaced valleys, and wherein, as the medical device is passed through the sheath, the ridges and valleys at least partially level out to allow a sheath wall to radially expand.

12. The expandable sheath of claim 1, wherein the first polymeric layer is bonded or adhered to at least a portion of the second surface of the inner liner, and the second polymeric layer is bonded or adhered to at least a portion of the first surface of the outer liner.

13. The expandable sheath of claim 12, wherein when the one or more polymer layers of the inner liner comprise a porous film, at least a portion of the first polymeric layer extends within at least a portion of the porous film of the inner liner; and/or when the one or more polymer layers of the outer liner comprise a porous film, at least a portion of the second polymeric layer extends within at least a portion of the porous film of the outer liner.

14. The expandable sheath of claim 1, wherein the braided layer comprises a plurality of helical multifilar filaments braided together, and,
wherein the braided layer has a proximal end and a distal end, wherein the braided layer comprises a plurality of closed loops at the distal end of the braided layer, and wherein the proximal end of the braided layer is positioned along a proximal end of the sheath.

15. The expandable sheath of claim 14, wherein the braided layer has a length extending from the proximal end of the braided layer to the distal end of the braided layer and wherein the braided layer length is shorter than a length of the sheath measured from a proximal end of the sheath to the distal end of the sheath.

16. The expandable sheath of claim 15, wherein the filaments of the braided layer are movable between the first and second polymeric layers such that the braided layer is configured to radially expand as a medical device is passed through the sheath while the length of the sheath remains substantially constant; and/or wherein the filaments of the braided layer are resiliently buckled when the sheath is in a collapsed configuration, and the first and second polymeric layers are attached to each other at a plurality of open spaces between the filaments of the braided layer.

17. A method of making an expandable sheath, the method comprising:
forming an inner liner; wherein the inner liner comprises one or more polymer layers and wherein the inner liner has a first surface and an opposite second surface;
forming a first polymeric layer, wherein the first polymeric layer is positioned radially outward of the inner liner; wherein the first polymeric layer comprises one or more sublayers; and wherein the first polymeric layer overlies the second surface of the inner liner;
positioning a braided layer radially outward of a first polymeric layer;
forming a second polymeric layer such that it is positioned radially outward of the braided layer; wherein the second polymeric layer comprises one or more sublayers;
forming an outer liner radially outward of the second polymeric layer; wherein the outer liner comprises one or more polymer layers, and wherein the outer liner has a first surface and an opposite second surface, and wherein the first surface of the outer liner is in contact with at least a portion of the second polymeric layer;
heating the inner liner, the first polymeric layer, the braided layer, the second polymeric layer, and the outer liner to form a laminate structure; and
crimping the laminate structure to form a plurality of longitudinally-extending pleats, wherein the plurality of longitudinally-extending pleats are configured to expand upon passage of a medical device through the sheath.

18. The method of claim 17, wherein
i) the step of forming the inner liner comprises wrapping a first material around an initial mandrel;
ii) the step of forming the first polymeric layer comprises wrapping a second material radially outward of the second surface of the inner liner, or the step of forming the first polymeric layer comprises coating of the second surface of the inner liner with one or more layers of a second material to form the one or more sublayers of the first polymeric layer;
iii) the step of forming the second polymeric layer comprises wrapping a third material outward of the braided layer, or the step of forming the second polymeric layer comprises coating the braided layer with one or more layers of a third material to form the one or more sublayers of the first polymeric layer; and/or
iv) the step of forming the outer liner comprises wrapping a fourth material radially outward of the second surface of the second polymeric layer.

19. The method of claim 17, wherein after forming the laminate structure, the method further comprises forming a third polymeric layer at at least a portion of a proximal end of the sheath, and wherein the step of forming the third polymeric layer comprises wrapping a fifth material radially outward of an outer surface of the outer liner, forming one or more polymer layers of the third polymeric layer.

20. The method of claim 18, wherein the step of heating comprises at least partially encapsulating the braided layer within the inner liner, the first polymeric layer, the second polymer layer, and the outer liner and/or wherein the step of heating comprises the second material at least partially penetrating a porous first material.
